(19) United States
(12) Patent Application Publication
(10) Pub. No.: US 2018/0000918 A1
(43) Pub. Date: Jan. 4, 2018

(54) VACCINE COMPOSITIONS FOR USE AGAINST ENTEROTOXIGENIC ESCHERICHIA COLI

(71) Applicant: Washington University, St. Louis, MO (US)

(72) Inventor: James M. FLECKENSTEIN, St. Louis, MO (US)

(21) Appl. No.: 15/543,487

(22) PCT Filed: Jan. 14, 2016
(86) PCT No.: PCT/US2016/013377

§ 371 Date: Jul. 13, 2017

Related U.S. Application Data
(60) Provisional application No. 62/103,549, filed on Jan. 14, 2015.

(51) Int. Cl. A61K 39/108; A61K 39/00

(52) U.S. Cl. CPC A61K 39/0258; A61K 2039/55544; A61K 2039/575

(57) ABSTRACT

The present disclosure provides vaccine compositions for use against enterotoxigenic Escherichia coli (ETEC) comprising EtpA and EatA. EtpA and EatA are prevalent amount diverse ETEC isolates and display significant sequence conservation. In an aspect, the disclosure encompasses a vaccine composition comprising EtpA and EatA. In another aspect, the disclosure encompasses a method of protecting against intestinal colonization of enterotoxigenic Escherichia coli (ETEC) in a subject. The method comprises administering to the subject an effective amount of a vaccine composition comprising EatA and EtpA.

### CROSS REFERENCE TO RELATED APPLICATIONS

This application claims the benefit of U.S. Provisional Application No. 62/103,549, filed Jan. 14, 2015, the disclosure of which is hereby incorporated by reference in its entirety.

### GOVERNMENTAL RIGHTS

This invention was made with government support under R01A1089894 awarded by the NIH. The government has certain rights in the invention.

### FIELD OF THE INVENTION

The present disclosure provides vaccine compositions for use against enterotoxigenic Escherichia coli (ETEC) comprising EtpA and EatA. EtpA and EatA are prevalent amount diverse ETEC isolates and display significant sequence conservation.

### BACKGROUND OF THE INVENTION

The enterotoxigenic Escherichia coli (ETEC) are among the most common causes of infectious diarrhea worldwide. Importantly, ETEC are disproportionately represented in cases of severe diarrheal illness as well as in deaths due to diarrhea among young children in developing countries [1].

These pathogens cause diarrhea by the elaboration and effective delivery of heat-labile and/or heat-stable enterotoxins to intestinal epithelial cells where they stimulate production of cyclic nucleotides ultimately activating the cystic fibrosis transmembrane regulator (CFTR) with resulting net efflux of fluid into the intestinal lumen [2]. Plasmid-encoded colonization factors (CFs), discovered [3] shortly after these organisms were identified as a causative agent of cholera-like diarrheal illness [4-6], are thought to be essential for effective colonization of the small intestine and required for ETEC pathogenesis.

Following early studies suggesting a pivotal role for these structures [7,8], CF antigens have defined the basis for most subsequent ETEC vaccine efforts [9,10]. However, one factor complicating development of a broadly protective vaccine for ETEC has been the general plasticity of E. coli genomes [11], and the significant antigenic heterogeneity of the CFs. To date, at least 26 antigenically distinct CF antigens have been described [12]. The lack of appreciable cross-protection afforded by these antigens combined with the complex landscape of CFs portrayed in ETEC molecular epidemiology studies continue to complicate rational CF antigen selection [13].

Antigenic heterogeneity, recent failure of LT-toxoid-based vaccine strategies [14,15], as well as the need to optimize the performance of live-attenuated vaccines currently in clinical trials [16-18] have highlighted the need to identify additional virulence molecules that might be targeted in ETEC vaccines.

### SUMMARY OF THE INVENTION

In an aspect, the disclosure encompasses a vaccine composition comprising EtpA and EatA.

In another aspect, the disclosure encompasses a method of protecting against intestinal colonization of enterotoxigenic Escherichia coli (ETEC) in a subject. The method comprises administering to the subject an effective amount of a vaccine composition comprising EatA and EtpA.

In still another aspect, the disclosure encompasses a method of preventing or treating ETEC-associated diarrhea. The method comprises administering to the subject an effective amount of a vaccine composition comprising EatA and EtpA.

In still another aspect, the disclosure encompasses a method of preventing or treating an ETEC-associated infection in a subject. The method comprises administering to the subject an effective amount of a vaccine composition comprising EatA and EtpA.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts the relationship of strain subsets used in antigen expression studies, and strains with available whole genome sequences. All of the strains in the collection (n=181) were examined for production of three secreted ETEC virulence proteins EtpA, EatA, and YghJ by immunoblotting of culture supernatants with the respective antibodies. A subset of these strains (n=91) were recently sequenced at the Genome Sequencing Center for Infectious Diseases (GSCID).

FIG. 2A, FIG. 2B and FIG. 2C depict the conservation of novel pathotype-specific antigens EtpA and EatA among phylogenically distinct strains expressing different colonization factors. (FIG. 2A) Heatmap of EtpA and EatA showing the proportion of strains positive for expression of these antigens among different CF groups. CF antigen designation is shown at left of the heatmap. nd=no CF antigen detected. Below is the heatmap key depicting colors associated with each degree of antigen positivity. Density line in yellow depicts the relative number of map features assigned at each proportion. Primary data used to construct the heatmap can be found in Table 5. (FIG. 2B) Immunoblot detection of EtpA and EatA expression among strains from different phylogenies. The upper immunoblot demonstrates EtpA production in the prototype H10407 strain, ThroopD isolated in Dallas, Tex. in 1975, the Juruá_18/11 (Amazon, 1998), and phylogentically dispersed strains from icddr,b. The etpA mutant is included as a negative control. The lower blot demonstrates EatA production by H10407, phylogenically distributed strains from icddr,b and Envira_10/1, an additional isolate from cholera-like outbreaks in the Amazon. The eatA mutant is included as a negative control. (FIG. 2C) Phlyogram showing the phylogenetic distribution of selected ETEC strains (designations in blue) and reference E. coli strains (designations in black). Red circles and gold stars represent eatA+, and etpA+ strains, respectively.

FIG. 3A, FIG. 3B, FIG. 3C and FIG. 3D depict recognition of novel antigens during naturally occurring ETEC infections in Bangladesh. Shown are kinetic ELISA data for four different recombinant antigens (FIG. 3A, rEtpA; FIG. 3B, the rEatA passenger domain; FIG. 3C, rYghJ; and FIG. 3D, rEaeH) obtained with 1:4096 dilutions of convalescent plasma from ETEC-infected patients hospitalized at ICDDR,B in Dhaka, Bangladesh (closed circles), or control patients not infected with enterotoxigenic E. coli (open circles). Horizontal bars represent geometric mean Vₘₐₓ kinetic ELISA values for each group. P values obtained by two-tailed Mann Whitney testing of groups are summarized (*<0.05; **<0.01; ***<0.001;****<0.0001). x-axis of each graph depicts the specificity of the secondary antibody used in the ELISA (IgG, IgA, IgM, and total IgG, IgA, and IgM).

FIG. 4A, FIG. 4B, FIG. 4C and FIG. 4D depict mice immunized with rEtpA and rEatAₚH134R are protected against ETEC infection. Serologic responses to (FIG. 4A) heat-labile toxin (LT), (FIG. 4B) the passenger domain of EatA (Eatₚ), (FIG. 4C) EtpA. Shown are serum IgG responses following intranasal vaccination of mice with the LT adjuvant alone, or LT with 15 μg of either the proteolytically inactive passenger domain (EatAₚH134R), EtpA, or both antigens on days 0, 14, 28. Colonization of mice following immunization with the adjuvant alone compared with single and dual antigen vaccination. Comparisons between groups were by Mann Whitney two tailed nonparametric testing. (One mouse died during the vaccination period in the LT/EtpA group and was therefore excluded from the analysis). For mice with no detectable colonies following challenge, the number of cfu is arbitrarily reported as 1(10°) cfu, the theoretical limit of detection.

FIG. 5A, FIG. 5B, FIG. 5C, FIG. 5D, FIG. 5E, FIG. 5F and FIG. 5G depict EatA passenger domain alignments of predicted EatA sequences corresponding to multiple phylogenies from strains belonging to disparate geographic origins. The conserved catalytic triad at amino acids H78, D106, and S211 is highlighted by gray background shading. Geographic origin of strains is depicted in the color key at left of the alignment. Alignments were performed using CLUSTAL Omega (release 1.2.0 AndreaGiacomo) [40] algorithm plugin for CLC Main Workbench. H10407 (SEQ ID NO:2); ThroopD (SEQ ID NO:16); Envira (10_1) (SEQ ID NO:17); Jurua (18_11) (SEQ ID NO:18); 2850750 (SEQ ID NO:19); 2871950 (SEQ ID NO:20); P03050293.1 (SEQ ID NO:17); P0304777.1 (SEQ ID NO:21); 2720900 (SEQ ID NO:22); 178900 (SEQ ID NO:23); 180200 (SEQ ID NO:19); 272950 (SEQ ID NO:16); TW10598 (SEQ ID NO:20); TW11681 (SEQ ID NO:16); B2C (SEQ ID NO:20); E24377A (SEQ ID NO:24); Consensus (SEQ ID NO:25).

FIG. 6A, FIG. 6B, FIG. 6C, FIG. 6D and FIG. 6E depict EtpA alignments of predicted sequences corresponding to multiple phylogenies from strains belonging to disparate geographic origins. Geographic origin of strains is depicted in the color key at left of the alignment. Alignments were performed using sequence alignment algorithm of CLC Main Workbench v6.9.1 with the following parameters: gap open cost=10.0; gap extension cost=1.0; end gap cost=as any other; alignment mode=very accurate (slow); redo alignments=no; use fixpoints=no. H10407 (SEQ ID NO:4); ThroopD (SEQ ID NO:26); Jurua (18/11) (SEQ ID NO:27); 2720900 (SEQ ID NO:4); 2850750 (SEQ ID NO:28); 2871950 (SEQ ID NO:29); P0302308.1 (SEQ ID NO:30); P0304777.1 (SEQ ID NO:31); P0299438.4 (SEQ ID NO:32); 178900 (SEQ ID NO:26); 180200 (SEQ ID NO:28); 1392/75 (SEQ ID NO:29); tw11681 (SEQ ID NO:26); tw14425 (SEQ ID NO:30); TW10828 (SEQ ID NO:28); tw10598 (SEQ ID NO:29); E24377A (SEQ ID NO:29).

FIG. 7A, FIG. 7B, FIG. 7C and FIG. 7D depict the immune response to selected ETEC proteins in infected patients and Bangledeshi and US controls. Shown are (IgG) kinetic ELISA responses (in Vₘₐₓ, milliunits/min) to recombinant proteins comparing convalescent plasma from patients hospitalized with acute ETEC infections at the International Centre for Diarrhoeal Disease Research in Dhaka, Bangladesh with controls (c) from Bangledeshi adults, and children, as well as plasma from age-matched children from Saint Louis Children's Hospital (slch). Antigens included two plasmid-encoded ETEC specific antigens (FIG. 7A) EtpA, and (FIG. 7B) the EatA passenger domain; and two chromosomally-encoded conserved antigens (FIG. 7C) YghJ, and (FIG. 7D) EaeH. All plasma samples were diluted 1:4096.

FIG. 8 depicts immune responses to EtpA following volunteer challenge with ETEC H10407. All sera were diluted 1:4096 prior to testing against rEtpA-myc-₆His followed by detection of total antibody (IgM,IgG,IgA) in kinetic ELISA. Pre and post values (open and closed circles, respectively) represent collective data from 2 independent ETEC H10407 challenge studies CIR218 and CIR193a. Data from CIR218 are shown as pre-challenge (d-2, open blue circles) and (d28, closed blue circles), while data from CIR193a appear as open grey circles (pre-challenge, dO) and closed grey circles (post challenge, d9). Dashed horizontal lines represent geometric means. P value represents comparison of pre and post-challenge samples by Mann Whitney 2-tailed analysis.

### DETAILED DESCRIPTION OF THE INVENTION

The present disclosure encompasses vaccine compositions and methods for preventing and treating ETEC-associated infections. The vaccine compositions comprise EatA and EtpA. The inventors demonstrated that EatA and EtpA are broadly represented in a diverse collection of ETEC isolates comprising multiple phylogenetic backgrounds. The prevalence and sequence conservation of EatA and EtpA among ETEC isolates makes them exceptional vaccine candidates. Importantly, vaccine targets should be specific to the pathovar under study or restricted to pathogenic isolates, but not subject to significant antigenic variation. These features are demonstrated by EatA and EtpA.

### I. Compositions

Compositions of the invention are directed to vaccine compositions comprising EtpA and EatA. In certain embodiments, the EatA protein comprises a mutation that disrupts its enzymatic activity. Various aspects of the invention will be described in further detail below.

### (a) EatA

In an aspect, the present disclosure encompasses a vaccine composition comprising EatA. The term “vaccine composition” as used herein means a composition that when administered to a subject, typically elicits a protective immune response, where a protective immune response is one that ameliorates one or more symptoms of the target disorder. As used herein, “EatA” refers to the enterotoxigenic E. coli autotransporter A. EatA is encoded by the eatA gene. EatA is a member of a family of molecules referred to as serine protease autotransporters of the Enterobacteriaceae (SPATE). EatA modulates both adherence to epithelial cells and intestinal colonization in part by digesting EtpA. Additionally, EatA degrades MUC2, the major mucin secreted by intestinal epithelium. EatA comprises a passenger domain. As used herein, the “passenger domain” comprises a HDS (histidine-aspartate-serine) catalytic triad and a C-terminal β-domain required for extracellular secretion of the passenger domain. A skilled artisan would be able to identify the passenger domain in EatA from various species or strains based on the recited features.

The nucleotide sequence of eatA may be found at GenBank accession number AY163491.2. The amino acid sequence of EatA may be found at GenBank accession number AAO17297.1. Homologs can be found in other species or strains by methods known in the art. For example, sequence similarity may be determined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the invention. Equally, BLAST protein searches may be performed with the BLASTX program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed. See www.ncbi.nlm.nih.gov for more details. Generally a homolog will have a least 80, 81, 82, 83, 84, 85, 86, 87, 88, or 89% homology. In another embodiment, the sequence may be at least 90, 91, 92, 93, 94, 95, 96, 97, 98, 99, or 100% homologous to EatA.

In a specific embodiment, an EatA of the vaccine composition comprises the full length sequence of ETEC H10407 EatA such as the sequence set forth in SEQ ID NO:1 (MNKVFSLKYS FLAKGFIAVS ELARRVSVKG KLKSASSIII SPITIAIVSY APPSLAATVN ADISYQTFRD FAENKGAFIV GASNINIYDK NGVLVGVLDK APMPDFSSAT MNTGTLPPGD HTLYSPQYW TAKHVNGSDI MSFGHIQNNY TVVGENNHNS LDIKIRRLNK IVTEVAPAEI SSVGAVNGAY QEGGRFKAFY RLGGGLQYIK DKNGNLTPVY TNGGFLTGGT ISALSSYNNG QMITAPTGDI FNPANGPLAN YLNKGDSGSP LFAYDSLDKK WVLVGVLSSG SEHGNNWVVT TQDFLHQQPK HDFDKTISYD SEKGSLQWRY NKNSGVGTLS QESVVWDMHG KKGGDLNAGK NLQFTGNNGE IILHDSIDQG AGYLQFFDNY TVTSLTDQTW TGGGIITEKG VNVLWQVNGV NDDNLHKVGE GTLTVNGKGV NNGGLKVGDG TVILNQRPDD NGHKQAFSSI NISSGRATVI LSDANQVNPD KISWGYRGGT LDLNGNNVNF TRLQAADYGA IVSNNNKNKS ELTLKLQTLN ENDISVDVKT YEVFGGHGSP GDLYYVPASN TYFILKSKAY GPFFSDLDNT NVWQNVGHDR DKAIQIVKQQ KIGESSQPYM FHGQLNGYMD VNIHPLSGKD VLTLDGSVNL PEGVITKKSG TLIFQGHPVI HAGMTTSAGQ SDWENRQFTM DKLRLDAATF HLSRNAHMQG DISAANGSTV ILGSSRVFTD KNDGTGNAVS SVEGSSIATT AGDQSYYSGN VLLENHSSLE VRENFTGGIE AYDSSVSVTS QNAIFDHVGS FVNSSLLLEK GAKLTAQSGI FTNNTMKIKE NASLTLTGIP SVGKPGYYSP VTSTTEGIHL GERASLSVKN MGYLSSNITA ENSAAIINLG DSNATIGKTD SPLFSTLMRG YNAVLQGNIM GPQSSVNMNN ALWHSDRNSE LKELKANDSQ IELGVRGHFA KLRVKELIAS NSVFLVHANN SQADQLNVTD KLQGSNNTIL VDFFNKAANG TNVTLITAPK GSDENTFKAG TQQIGFSNIT PEIRTENTDT ATQWVLTGYQ SVADARASKI ATDFMDSGYK SFLTEVNNLN KRMGDLRDSQ GDAGGWARIM NGTGSGESGY RDNYTHVQIG ADRKHELNGI DLFTGALLTY TDNNASSQAF SGKTKSLGGG VYASGLFESG AYFDLIGKYL HHDNRYTLNF ASLGERSYTS HSLYAGAEIG YRYHMSENTW VEPQMELVYG SVSGKSFNWK DQGMQLSMKD KDYHPLIGRT GVDVGRAFSG DTWKVTVRAG LGYQFDLLAN GETVLQDASG KKHFKGEKDS RMLMNVGTNV EVKDNMRFGL ELEKSAFGRY NIDNSINANF RYYF).

In another specific embodiment, an EatA of the vaccine composition comprises the passenger domain of ETEC H10407 EatA such as the sequence set forth in SEQ ID NO:2 (ATVN ADISYQTFRD FAENKGAFIV GASNINIYDK NGVLVGVLDK APMPDFSSAT MNTGTLPPGD HTLYSPQYW TAKHVNGSDI MSFGHIQNNY TVVGENNHNS LDIKIRRLNK IVTEVAPAEI SSVGAVNGAY QEGGRFKAFY RLGGGLQYIK DKNGNLTPVY TNGGFLTGGT ISALSSYNNG QMITAPTGDI FNPANGPLAN YLNKGDSGSP LFAYDSLDKK WVLVGVLSSG SEHGNNWVVT TQDFLHQQPK HDFDKTISYD SEKGSLQWRY NKNSGVGTLS QESVVWDMHG KKGGDLNAGK NLQFTGNNGE IILHDSIDQG AGYLQFFDNY TVTSLTDQTW TGGGIITEKG VNVLWQVNGV NDDNLHKVGE GTLTVNGKGV NNGGLKVGDG TVILNQRPDD NGHKQAFSSI NISSGRATVI LSDANQVNPD KISWGYRGGT LDLNGNNVNF TRLQAADYGA IVSNNNKNKS ELTLKLQTLN ENDISVDVKT YEVFGGHGSP GDLYYVPASN TYFILKSKAY GPFFSDLDNT NVWQNVGHDR DKAIQIVKQQ KIGESSQPYM FHGQLNGYMD VNIHPLSGKD VLTLDGSVNL PEGVITKKSG TLIFQGHPVI HAGMTTSAGQ SDWENRQFTM DKLRLDAATF HLSRNAHMQG DISAANGSTV ILGSSRVFTD KNDGTGNAVS SVEGSSIATT AGDQSYYSGN VLLENHSSLE VRENFTGGIE AYDSSVSVTS QNAIFDHVGS FVNSSLLLEK GAKLTAQSGI FTNNTMKIKE NASLTLTGIP SVGKPGYYSP VTSTTEGIHL GERASLSVKN MGYLSSNITA ENSAAIINLG DSNATIGKTD SPLFSTLMRG YNAVLQGNIM GPQSSVNMNN ALWHSDRNSE LKELKANDSQ IELGVRGHFA KLRVKELIAS NSVFLVHANN SQADQLNVTD KLQGSNNTIL VDFFNKAANG TNVTLITAPK GSDENTFKAG TQQIGFSNIT PEIRTENTDT ATQWVLTGYQ SVADARASKI ATDFMDSGYK SFLTEVNNLN KRMGDLRD).

In some embodiments, an EatA of the vaccine composition is a sequence of EatA comprising at least 80%, at least 81%, at least 82%, at least 83%, at least 84%, at least 85%, at least 86%, at least 87%, at least 88%, or 89% identity to SEQ ID NO:1 or SEQ ID NO:2. In another embodiment, an EatA of the vaccine composition is a sequence of EatA comprising at least 90%, at least 91%, at least 92%, at least 93%, at least 94%, at least 95%, at least 96%, at least 97%, at least 98%, at least 99%, or at least 100% identity to SEQ ID NO:1 or SEQ ID NO:2. In a specific embodiment, an EatA of the vaccine composition is a sequence of EatA comprising about 95% to about 100% identity to SEQ ID NO:1 or SEQ ID NO:2.

In a specific embodiment, an EatA of the vaccine composition comprises a mutation that disrupts the serine protease activity. Methods known in the art may be used to determine if a mutation in EatA results in disruption in serine protease activity. For example, the ability of mutated EatA to cleave substrate may be determined. EatA possesses serine protease activity that is abolished by mutations within a serine protease catalytic triad formed by residues H134, D162, and S267. The catalytic triad is universally conserved within the passenger domain of EatA. Accordingly, an EatA of the vaccine composition may comprise one or more mutations in H134, D162, and/or S267, wherein the mutation disrupts serine protease activity. More specifically, an EatA of the vaccine composition may comprise one or mutations selected from the group consisting of H134A, D162A, and S267G, relative to SEQ ID NO:1.

In some embodiments, an EatA of the vaccine composition is a sequence of EatA comprising a mutation that disrupts the serine protease activity and has at least 80%, at least 81%, at least 82%, at least 83%, at least 84%, at least 85%, at least 86%, at least 87%, at least 88%, or 89% identity to SEQ ID NO:1 or SEQ ID NO:2. In another embodiment, an EatA of the vaccine composition is a sequence of EatA comprising a mutation that disrupts the serine protease activity and has at least 90%, at least 91%, at least 92%, at least 93%, at least 94%, at least 95%, at least 96%, at least 97%, at least 98%, at least 99%, or at least 100% identity to SEQ ID NO:1 or SEQ ID NO:2. In a specific embodiment, an EatA of the vaccine composition is a sequence of EatA comprising a mutation that disrupts serine protease activity and has about 95% to about 100% identity to SEQ ID NO:1 or SEQ ID NO:2.

In other embodiments, an EatA of the vaccine composition is a sequence of EatA comprising one or more mutations in H134, D162, and/or S267 that disrupts the serine protease activity and has at least 80%, at least 81%, at least 82%, at least 83%, at least 84%, at least 85%, at least 86%, at least 87%, at least 88%, or 89% identity to SEQ ID NO:1 or SEQ ID NO:2. In another embodiment, an EatA of the vaccine composition is a sequence of EatA comprising one or more mutations in H134, D162, and/or S267 that disrupts the serine protease activity and has at least 90%, at least 91%, at least 92%, at least 93%, at least 94%, at least 95%, at least 96%, at least 97%, at least 98%, at least 99%, or at least 100% identity to SEQ ID NO:1 or SEQ ID NO:2. In a specific embodiment, an EatA of the vaccine composition is a sequence of EatA comprising one or more mutations in H134, D162, and/or S267 that disrupts serine protease activity and has about 95% to about 100% identity to SEQ ID NO:1 or SEQ ID NO:2.

In still other embodiments, an EatA of the vaccine composition is a sequence of EatA comprising one or more mutations selected from the group consisting of H134A, D162A, and S267G and has at least 80%, at least 81%, at least 82%, at least 83%, at least 84%, at least 85%, at least 86%, at least 87%, at least 88%, or 89% identity to SEQ ID NO:1 or SEQ ID NO:2. In another embodiment, an EatA of the vaccine composition is a sequence of EatA comprising one or more mutations selected from the group consisting of H134A, D162A, and S267G that disrupts the serine protease activity and has at least 90%, at least 91%, at least 92%, at least 93%, at least 94%, at least 95%, at least 96%, at least 97%, at least 98%, at least 99%, or at least 100% identity to SEQ ID NO:1 or SEQ ID NO:2. In a specific embodiment, an EatA of the vaccine composition is a sequence of EatA comprising one or more mutations selected from the group consisting of H134A, D162A, and S267G that disrupts serine protease activity and has about 95% to about 100% identity to SEQ ID NO:1 or SEQ ID NO:2.

In any of the foregoing embodiments, an EatA of the vaccine composition may be a truncated version of EatA provided it as the same activity as the full length or passenger domain of EatA (e.g. elicits a protective immune response or, stated another way, is antigenic). For example, the truncated version of EatA may be about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800, about 810, about 820, about 830, about 840, about 850, about 860, about 870, about 880, about 890, about 900, about 910, about 920, about 930, about 940, about 950, about 960, about 970, about 980, about 990, about 1000, about 1010, about 1020, about 1030, about 1040, about 1050, about 1060, about 1070, about 1080, about 1090, about 1100, about 1110, about 1120, about 1130, about 1140, about 1150, about 1160, about 1170, about 1180, about 1190, about 1200, about 1210, about 1220, about 1230, about 1240, about 1250, about 1260, about 1270, about 1280, about 1290, about 1300, about 1310, about 1320, about 1330, about 1340, about 1350, about 1360, or about 1370 amino acids, provided it as the same activity as the full length or passenger domain of EatA (elicits a protective immune response or, stated another way, is antigenic).

### (b) EtpA

In an aspect, the present disclosure encompasses a vaccine composition comprising EtpA. As used herein, “EtpA” refers to the ETEC two-partner secretion locus (etpBAC) protein A and is encoded by the etpA gene. EtpA is a member of a family of virulence proteins (generically referred to as TpsA proteins) that are secreted by two-partner secretion (TPS). EtpA is an exoprotein adhesin molecule and plays a critical role in bacterial adhesion in vitro and in the colonization of mucosal surfaces in vivo. EtpA has a conserved secretion domain in its amino terminus comprising Asn-Pro-Asn-Gly-Val (SEQ ID NO:5) at amino acids 150 to 154 and several repeat regions in the carboxy-terminus comprising four major repeat units (˜226 amino acids) preceded by a 173-amino-acid partial repeat beginning at amino acid S648.

The nucleotide sequence of etpA may be found at GenBank accession number AY920525.2. The amino acid sequence of EtpA may be found at GenBank accession number AAX13509.2. Homologs can be found in other species or strains by methods known in the art. For example, sequence similarity may be determined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the invention. Equally, BLAST protein searches may be performed with the BLASTX program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed. See www.ncbi.nlm.nih.gov for more details. Generally a homolog will have a least 80, 81, 82, 83, 84, 85, 86, 87, 88, or 89% homology. In another embodiment, the sequence may be at least 90, 91, 92, 93, 94, 95, 96, 97, 98, 99, or 100% homologous to EtpA.

In a specific embodiment, an EtpA of the vaccine composition comprises the full length sequence of ETEC H10407 EtpA such as the sequence set forth in SEQ ID NO:3 (MNRIYKLKFD KRRNELVWS EITTGVGNAK ATGSVEGEKS PRRGVRAMAL SLLSGMMIMA HPAMSANLPT GGQIVAGSGS IQTPSGNQMN IHQNSQNMVA NWNSFDIGKG NTVQFDQPSS SAVALNRWG GGESQIMGNL KANGQVFLVN PNGVLFGEGA SVSTSGFVAS TRDIKNDDFM NRRYTFSGGQ KAGAAIVNQG ELTTNAGGYI VLAADRVSNS GTIRTPGGKT VLAASERITL QLDNGGLMSV QVTGDVVNAL VENRGLVSAR DGQVYLTALG RGMLMNTVLN VSGWEASGM HRQDGNIVLD GGDSGVVHLS GTLQADNASG QGGKVVVQGK NILLDKGSNI TATGGQGGGE VYVGGGWQGK DSNIRNADKV VMQGGARIDV SATQQGNGGT AVLWSDSYTN FHGQISAKGG ETGGNGGRVE TSSHGNLQAF GTVSASAKKG KAGNWLLDSA DITIVNGSNV SKTETTQSPP HTQFAPTAAG SAVSNTSINN RLNNGTSVTI LTHRTRTGTA QGGNITVNAA INKSNGSDVN LTLQAGGNIT VNNSITSTEG KLNVNLSGAR TSNGSITISN NANITTNGGD ITVGTTNTSN RVNISINNTT LNASNGNIQL TGTGTDSGIL FAGNNRLTAS NIALTGNSTS GNAINLTGTA TLNATNNITL TGSSTSGNAI NLKGNNTLTA SNITLTGEST SGNAINLTDT TGTTTLNATN NITMQGTRVQ IKHSNITAGN FALNATVAGS EISNTTLTAT NNINLAAKTN SASSGVYLKD ARITSTNGSI TANGTATANG KATHLDGNVT LNASNGRIKL TGNGHGSASG ILFAGNNRLT ASNIALTGNS TSGNAINLTG TATLNATNDI TLTGSSTSGN AINLTGTATL NATNNITLTG SSTSGNAINL KGNNTLTASN ITLTGESTSG NAINLTDTTG TTTLNATNNI TMQGTRVQIK HSNITAGNFA LNATVAGSEI SNTTLTATNN INLAAKTNSA SSGVYLKDAR ITSTNGSITA NGTATANGKA THLDGNVTLN ASNGRIKLTG NGHGSASGIL FAGNNRLTAS NIALTGNSTS GNAINLTGTA TLNATNDITL TGSSTSGNAI NLTGTATLNA TNNITLTGSS TSGNAINLKG NNTLTASNIT LTGESTSGNA INLTDTTGTT TLNATNNITM QGTRVQIKHS NITAGNFALN ATVAGSEISN TTLTATNNIN LAAKTNSASS GVYLKDARIT STNGSITANG TATANGKATH LDGNVTLNAS NGRIKLTGNG HGSASGILFA GNNRLTASNI ALTGNSTSGN AINLTGTATL NATNDITLTG SSTSGNAINL TGTATLNATN NITLTGSSTS GNAINLKGNN TLTASNITLT GESTSGNAIN LTDTTGTTTL NATNNITMQG TRVQIKHSNI TAGNFALNAT VAGSEISNTT LTATNNINLA AKTNSASSGV YLKDARITST NGSITTNGTA TANGKATHLD GNVTLNASNG RIKLTGNGHG SASGILFAGN NRLTASNIAL TGNSTSGNAI NLTGTATLNA TNDITLTGSS TSGNAINLTG TATLNATNNI TLTGSSTSGN AINLKGNNTL TASNITLTGE STSGNAINLT DTTGTTTLNA TNNITMQGTR VQIKHSNITA GNFALNATVA GSEISNTTLT ATNNINLAAK TNSASSGVYL KDARITSTNG SITANGTAPA NDNATYLDGN VTLNASNGSI KLTGNGNGST SGILFAGNNT LTASNITLTG NSEVYWQ).

In another specific embodiment, an EtpA of the vaccine composition comprises the secreted portion of ETEC H10407 EtpA such as the sequence set forth in SEQ ID NO:4 (MNRIYKLKFD KRRNELVWS EITTGVGNAK ATGSVEGEKS PRRGVRAMAL SLLSGMMIMA HPAMSANLPT GGQIVAGSGS IQTPSGNQMN IHQNSQNMVA NWNSFDIGKG NTVQFDQPSS SAVALNRVVG GGESQIMGNL KANGQVFLVN PNGVLFGEGA SVSTSGFVAS TRDIKNDDFM NRRYTFSGGQ KAGAAIVNQG ELTTNAGGYI VLAADRVSNS GTIRTPGGKT VLAASERITL QLDNGGLMSV QVTGDVVNAL VENRGLVSAR DGQVYLTALG RGMLMNTVLN VSGWEASGM HRQDGNIVLD GGDSGWHLS GTLQADNASG QGGKVVVQGK NILLDKGSNI TATGGQGGGE VYVGGGWQGK DSNIRNADKV VMQGGARIDV SATQQGNGGT AVLWSDSYTN FHGQISAKGG ETGGNGGRVE TSSHGNLQAF GTVSASAKKG KAGNWLLDSA DITIVNGSNV SKTETTQSPP HTQFAPTAAG SAVSNTSINN RLNNGTSVTI LTHRTRTGTA QGGNITVNAA INKSNGSDVN LTLQAGGNIT VNNSITSTEG KLNVNLSGAR TSNGSITISN NANITTNGGD ITVGTTNTSN).

In some embodiments, an EtpA of the vaccine composition is a sequence of EtpA comprising at least 80%, at least 81%, at least 82%, at least 83%, at least 84%, at least 85%, at least 86%, at least 87%, at least 88%, or 89% identity to SEQ ID NO:3 or SEQ ID NO:4. In another embodiment, an EtpA of the vaccine composition is a sequence of EtpA comprising at least 90%, at least 91%, at least 92%, at least 93%, at least 94%, at least 95%, at least 96%, at least 97%, at least 98%, at least 99%, or at least 100% identity to SEQ ID NO:3 or SEQ ID NO:4. In a specific embodiment, an EtpA of the vaccine composition is a sequence of EtpA comprising about 94% to about 100% identity to SEQ ID NO:3 or SEQ ID NO:4.

In any of the foregoing embodiments, an EtpA of the vaccine composition may be a truncated version of EtpA provided it as the same activity as the full length or secreted portion of EtpA (elicits a protective immune response or, stated another way, is antigenic). For example, the truncated version of EtpA may be about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, about 490, about 500, about 510, about 520, about 530, about 540, about 550, about 560, about 570, about 580, about 590, about 600, about 610, about 620, about 630, about 640, about 650, about 660, about 670, about 680, about 690, about 700, about 710, about 720, about 730, about 740, about 750, about 760, about 770, about 780, about 790, about 800, about 810, about 820, about 830, about 840, about 850, about 860, about 870, about 880, about 890, about 900, about 910, about 920, about 930, about 940, about 950, about 960, about 970, about 980, about 990, about 1000, about 1010, about 1020, about 1030, about 1040, about 1050, about 1060, about 1070, about 1080, about 1090, about 1100, about 1110, about 1120, about 1130, about 1140, about 1150, about 1160, about 1170, about 1180, about 1190, about 1200, about 1210, about 1220, about 1230, about 1240, about 1250, about 1260, about 1270, about 1280, about 1290, about 1300, about 1310, about 1320, about 1330, about 1340, about 1350, about 1360, about 1370, about 1380, about 1390, about 1400, about 1410, about 1420, about 1430, about 1440, about 1450, about 1460, about 1470, about 1480, about 1490, about 1500, about 1510, about 1520, about 1530, about 1540, about 1550, about 1560, about 1570, about 1580, about 1590, about 1600, about 1610, about 1620, about 1630, about 1640, about 1650, about 1660, about 1670, about 1680, about 1690, about 1700, about 1710, about 1720, about 1730, about 1740, about 1750, about 1760, or about 1770 amino acids, provided it as the same activity as the full length or secreted portion of EtpA (elicits a protective immune response or, stated another way, is antigenic).

### (c) Combinations

In an embodiment, the vaccine composition may further comprise other immunogenic ETEC proteins. For example, the vaccine composition may further comprise colonization factor (CF) antigens. Colonization factors are proteinaceous surface appendages that facilitate adherence of bacteria to the intestinal epithelium of the host. Generally, CFs are fimbrial, fibrillar, or afimbrial structures. There are 22 different CF variants currently described (including colonization factor antigen I [CFA/I]), and the majority are usually designated “CS” followed by a number that indicates their placement in an order arranged according to the date of discovery. Non-limiting examples of CFs include CFA/I, CS1, CS2, CS3, CS4, CS5, CS6, CS7, CS8, CS9, CS10, CS11, CS12, CS13, CS14, CS15, CS16, CS17, CS18, CS19, CS20, CS21, CS22, and CS23. The vaccine composition may also further comprise nonclassical adhesins such as Tia and TibA.

### (d) Composition

In an aspect, a vaccine composition of the invention comprises EatA and EtpA. In an embodiment, a vaccine composition of the invention comprises EatA and EtpA linked together. The EatA and EtpA may be linked together by various methods known in the art. Suitable linkers include amino acid chains and alkyl chains functionalized with reactive groups for coupling to EatA and EtpA. In an embodiment, the linker may include amino acid side chains, referred to as a peptide linker. Amino acid residue linkers are usually at least one residue and can be 40 or more residues, more often 1 to 10 residues, but do not comprise EatA or EtpA. Typical amino acid residues used for linking are tyrosine, cysteine, lysine, glutamic and aspartic acid, or the like.

In another embodiment, an alkyl chain linking group may be coupled to EatA and EtpA by reacting the amino group of the N-terminal residue of EatA with a first functional group on the alkyl chain, such as a carboxyl group or an activated ester. Subsequently, EtpA is attached to the alkyl chain to complete the formation of the complex by reacting a second functional group on the alkyl chain with an appropriate group on EtpA. The second functional group on the alkyl chain is selected from substituents that are reactive with a functional group on EtpA while not being reactive with the N-terminal residue of EatA. The process may also be reversed.

An alternative chemical linking group to an alkyl chain is polyethylene glycol (PEG), which is functionalized in the same manner as the alkyl chain described above. The EatA and/or EtpA of the invention may be PEGylated for improved systemic half-life and reduced dosage frequency. In an embodiment, PEG may be added to a linker.

The vaccine compositions of the invention may include a pharmaceutically acceptable excipient such as a suitable adjuvant. Suitable adjuvants include an aluminum salt such as aluminum hydroxide or aluminum phosphate, but may also be a salt of calcium, iron or zinc, or may be an insoluble suspension of acylated tyrosine, or acylated sugars, or may be cationically or anionically derivatised saccharides, polyphosphazenes, biodegradable microspheres, monophosphoryl lipid A (MPL), lipid A derivatives (e.g. of reduced toxicity), 3-O-deacylated MPL [3D-MPL], quit A, Saponin, QS21, Freund's Incomplete Adjuvant (Difco Laboratories, Detroit, Mich.), Merck Adjuvant 65 (Merck and Company, Inc., Rahway, N.J.), AS-2 (Smith-Kline Beecham, Philadelphia, Pa.), CpG oligonucleotides, bioadhesives and mucoadhesives, microparticles, liposomes, polyoxyethylene ether formulations, polyoxyethylene ester formulations, muramyl peptides or imidazoquinolone compounds (e.g. imiquamod and its homologues). Human immunomodulators suitable for use as adjuvants in the invention include cytokines such as interleukins (e.g. IL-1, IL-2, IL-4, IL-5, IL-6, IL-7, IL-12, etc), macrophage colony stimulating factor (M-CSF), tumour necrosis factor (TNF), granulocyte, macrophage colony stimulating factor (GM-CSF) may also be used as adjuvants. In a specific embodiment, the adjuvant is heat-labile toxin (LT) or double mutant heat-labile toxin (LT).

Vaccines of the invention will typically, in addition to the antigenic and adjuvant components mentioned above, comprise one or more “pharmaceutically acceptable carriers or excipients”, which include any excipient that does not itself induce the production of antibodies harmful to the individual receiving the composition. Suitable excipients are typically large, slowly metabolised macromolecules such as proteins, saccharides, polylactic acids, polyglycolic acids, polymeric amino acids, amino acid copolymers, sucrose (Paoletti et al., 2001, Vaccine, 19:2118), trehalose (WO 00/56365), lactose and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, and the like, may be present. Sterile pyrogen-free, phosphate buffered physiologic saline is a typical carrier. A thorough discussion of pharmaceutically acceptable excipients is available in reference Gennaro, 2000, Remington: The Science and Practice of Pharmacy, 20^{th} edition, ISBN:0683306472.

Compositions of the invention may be lyophilised or in aqueous form, i.e. solutions or suspensions. Liquid formulations of this type allow the compositions to be administered direct from their packaged form, without the need for reconstitution in an aqueous medium, and are thus ideal for injection. Compositions may be presented in vials, or they may be presented in ready filled syringes. The syringes may be supplied with or without needles. A syringe will include a single dose of the composition, whereas a vial may include a single dose or multiple doses (e.g. 2 doses).

Liquid vaccines of the invention are also suitable for reconstituting other vaccines from a lyophilized form. Where a vaccine is to be used for such extemporaneous reconstitution, the invention provides a kit, which may comprise two vials, or may comprise one ready-filled syringe and one vial, with the contents of the syringe being used to reconstitute the contents of the vial prior to injection.

Vaccines of the invention may be packaged in unit dose form or in multiple dose form (e.g. 2 doses). For multiple dose forms, vials are preferred to pre-filled syringes. Effective dosage volumes can be routinely established, but a typical human dose of the composition for injection has a volume of 0.5 mL.

In one embodiment, vaccines of the invention have a pH of between 6.0 and 8.0, in another embodiment, vaccines of the invention have a pH of between 6.3 and 6.9, e.g. 6.6±0.2. Vaccines may be buffered at this pH. Stable pH may be maintained by the use of a buffer. If a composition comprises an aluminum hydroxide salt, a histidine buffer may be used (WO03/009869). The composition should be sterile and/or pyrogen free.

Compositions of the invention may be isotonic with respect to humans.

Vaccines of the invention may include an antimicrobial, particularly when packaged in a multiple dose format. Antimicrobials may be used, such as 2-phenoxyethanol or parabens (methyl, ethyl, propyl parabens). Any preservative is preferably present at low levels. Preservative may be added exogenously and/or may be a component of the bulk antigens which are mixed to form the composition (e.g. present as a preservative in pertussis antigens).

Vaccines of the invention may comprise a detergent e.g. a Tween (polysorbate), such as Tween 80. Detergents are generally present at low levels e.g. <0.01%.

Vaccines of the invention may include sodium salts (e.g. sodium chloride) to give tonicity. The composition may comprise sodium chloride. In one embodiment, the concentration of sodium chloride in the composition of the invention is in the range of 0.1 to 100 mg/mL (e.g. 1-50 mg/mL, 2-20 mg/mL, 5-15 mg/mL) and in a further embodiment the concentration of sodium chloride is 10±2 mg/mL NaCl e.g. about 9 mg/m L.

Vaccines of the invention will generally include a buffer. A phosphate or histidine buffer is typical.

Vaccines of the invention may include free phosphate ions in solution (e.g. by the use of a phosphate buffer) in order to favor non-adsorption of antigens. The concentration of free phosphate ions in the composition of the invention is in one embodiment between 0.1 and 10.0 mM, or in another embodiment between 1 and 5 mM, or in a further embodiment about 2.5 mM.

In other embodiments, the vaccine composition may be a live attenuated vaccine vector strain. A live attenuated vaccine vector strain is a strain that has reduced virulence but is still viable. In such an embodiment, EatA and EtpA are expressed in the attenuated strain. Importantly, in such an embodiment, the EatA comprises a mutation in a least two of the three catalytic triad amino acids. Additional inactivating mutations in EatA will limit the likelihood that a mutation in vivo will allow reversion. In certain embodiments, the live attenuated vaccine vector is Salmonella typhi Ty21a. In other embodiments, the live attenuated vaccine vector is any of the three attenuated strains of ACE527.

### II. Methods

In an aspect, the present disclosure encompasses a method of protecting against intestinal colonization of enterotoxigenic Escherichia coli (ETEC) in a subject. The method comprises administering to the subject an effective amount of a vaccine composition comprising EatA and EtpA. As used herein, “enterotoxigenic Escherichia coli” or “ETEC” is an E. coli strain able to produce at least one of two types of enterotoxins, the heat-labile toxin (LT) and/or the heat-stable toxin (ST). LT and/or ST are responsible for the movement of electrolytes and water from the intestinal cells to the intestinal lumen, resulting in watery diarrhea. Accordingly, the present disclosure also encompasses a method of preventing or treating ETEC-associated diarrhea.

In another aspect, the present disclosure encompasses a method of preventing or treating an ETEC-associated infection in a subject. The method comprises administering to the subject an effective amount of a vaccine composition comprising EatA and EtpA. The term “infection” as used herein includes presence of microbes, including bacteria, in or on a subject, which, if its growth were inhibited, would result in a benefit to the subject. As such, the term “infection” in addition to referring to the presence of bacteria also refers to normal flora which, are not desirable. The term “infection” includes infection caused by bacteria. An “ETEC-associated infection” is an infection caused by enterotoxigenic Escherichia coli (ETEC). Non-limiting examples of infections that may be prevented or treated using the compositions and/or methods of the invention include: infectious diarrhea, gastroenteritis or travelers' diarrhea.

The term “treat”, “treating” or “treatment” as used herein refers to administering a pharmaceutical composition of the invention for prophylactic and/or therapeutic purposes. The term “prophylactic treatment” refers to treating a subject who is not yet infected, but who is susceptible to, or otherwise at a risk of infection. The term “therapeutic treatment” refers to administering treatment to a subject already suffering from infection. The term “treat”, “treating” or “treatment” as used herein also refers to administering a pharmaceutical composition of the invention in order to: (i) reduce or eliminate either an ETEC-associated infection or one or more symptoms of the ETEC-associated infection, or (ii) retard the progression of an ETEC-associated infection or of one or more symptoms of the ETEC-associated infection, or (iii) reduce the severity of an ETEC-associated infection or of one or more symptoms of the ETEC-associated infection, or (iv) suppress the clinical manifestation of an ETEC-associated infection, or (v) suppress the manifestation of adverse symptoms of the ETEC-associated infection. Non-limiting examples of symptoms of an ETEC-associate infection include profuse watery diarrhea, abdominal cramping, fever, nausea with or without vomiting, chills, loss of appetite, headache, muscle aches and bloating.

The term “control” or “controlling” as used herein generally refers to preventing, reducing, or eradicating an ETEC-associated infection or inhibiting the rate and extent of such an infection, or reducing the microbial population, such as a microbial population present in or on a body or structure, surface, liquid, subject, etc, wherein such prevention or reduction in the ETEC-associated infection or microbial population is statistically significant with respect to untreated infection or population. In general, such control may be achieved by increased mortality amongst the microbial population.

The compositions of the present invention may be used to protect or treat a subject susceptible to infection by ETEC by means of administering said composition directly to a subject. The term “administration” or “administering” includes delivery of a composition or one or more pharmaceutically active ingredients to a subject, including for example, by any appropriate methods, which serves to deliver the composition or its active ingredients or other pharmaceutically active ingredients to the site of the infection. The method of administration can vary depending on various factors, such as for example, the components of the pharmaceutical composition or the type/nature of the pharmaceutically active or inert ingredients, the site of the potential or actual infection, the microorganism involved, severity of the infection, age and physical condition of the subject. Direct delivery may be accomplished by parenteral injection (intramuscularly, intraperitoneally, intradermally, subcutaneously, intravenously, or to the interstitial space of a tissue); or by rectal, oral, vaginal, topical, transdermal, intranasal, ocular, aural, pulmonary or other mucosal administration. In one embodiment, administration is by intramuscular injection to the thigh or the upper arm. Injection may be via a needle (e.g. a hypodermic needle, electroporation device), but needle free injection may alternatively be used. A typical intramuscular dose is 0.5 mL. In another embodiment, administration is intranasal administration. The composition can be administered prophylactically (i.e. to prevent infection) or therapeutically (i.e. to treat infection). An immune response is preferably protective. The method may raise a booster response.

The invention provides a method for preventing of treating ETEC-associated infection in a subject, comprising the step of administering an effective amount of a composition of the invention. The term “effective amount” as used herein refers to an amount, which has a therapeutic effect or is the amount required to produce a therapeutic effect in a subject. For example, a therapeutically or pharmaceutically effective amount of a composition is the amount of the antigen required to produce a desired therapeutic effect as may be judged by clinical trial results and/or model animal infection studies. The effective or pharmaceutically effective amount depends on several factors, including but not limited to, the route of administration, the microorganism (e.g. bacteria) involved, characteristics of the subject (for example height, weight, sex, age and medical history), severity of infection, location of infection, and/or the particular type of antigen used. For prophylactic treatments, a therapeutically or prophylactically effective amount is that amount which would be effective to prevent a microbial (e.g. bacterial) infection.

The effective amount of antigen in each vaccine dose is selected as an amount which induces an immunoprotective response without significant adverse side effects in typical vaccines. Accordingly, the exact amount of the antigen that is required to elicit such a response will vary from subject to subject, depending on the species, age, and general condition of the subject, the severity of the condition being treated, the particular carrier or adjuvant being used and its mode of administration, and the like. Generally it is expected that each dose will comprise 1-1000 μg of total antigen, or 1-100 μg, or 1-40 μg, or 1-5 μg, or less than 1 μg. An optimal amount for a particular vaccine can be ascertained by studies involving observation of antibody titres and other responses in subjects. In certain embodiments, the vaccine composition is administered at a dose ranging from about 50 to 150 μg. In another embodiment, the vaccine composition is administered at a dose of about 100 μg. In an exemplary embodiment, the vaccine composition is administered at a dose ranging from about 15 to about 30 μg.

Following initial administration of a vaccine composition of the disclosure, subjects may receive one or several additional administrations of the composition adequately spaced. Dosing treatment can be a single dose schedule or a multiple dose schedule. Suitable timing between doses (e.g. between 2-16 weeks) can be routinely determined.

In the prevention of an infection, a composition of the invention may be administered as multiple doses prior to infection. In the treatment of an infection, a composition of the invention may be administered as multiple doses following infection. Administration may be daily, twice daily, weekly, twice weekly, monthly, twice monthly, every 6 weeks, every 3 months, every 6 months or yearly. For example, administration may be every 2 weeks, every 3 weeks every 4 weeks, every 5 weeks, every 6 weeks, every 7 weeks, every 8 weeks, every 9 weeks, every 10 weeks, every 11 weeks or every 12 weeks. Alternatively, administration may be every 1 month, every 2 months, every 3 months, every 4 months, every 5 months, every 6 months, every 7 months, every 8 months, every 9 months, every 10 months, every 11 months or every 12 months. Still further, administration may be every 1 year, every 2 years, every 3 years, every 4 years, every 5 years, every 6 years, every 7 years, every 8 years, every 9 years, every 10 years, every 15 years or every 20 years. The duration of treatment can and will vary depending on the subject and the infection to be prevented or treated. For example, the duration of treatment may be for 1 week, 2 weeks, 3 weeks, 4 weeks, 5 weeks, 6 weeks. Alternatively, the duration of treatment may be for 1 month, 2 months, 3 months, 4 months, 5 months, 6 months, 7 months, 8 months, 9 months, 10 months, 11 months, or 12 months. In still another embodiment, the duration of treatment may be for 1 year, 2 years, 3 years, 4 years, 5 years, or greater than 5 years. It is also contemplated that administration may be frequent for a period of time and then administration may be spaced out for a period of time. For example, administration may be every 4 weeks for 6 months to a year and then administration may be every year thereafter. The duration of treatment may also depend on the length of time the subject may be exposed to ETEC. For example, when the subject resides in or visits a region endemic for ETEC, the duration of treatment may be based on the length of time the subject spends in that region. In a specific embodiment, the duration of treatment may be once a day for the duration of time the subject is in the endemic region. A skilled artisan would be able to determine the effective dosing regimen based on the medical history and subject characteristics.

A vaccine composition of the disclosure may be administered in combination with standard treatments for ETEC-associated infection. Non-limiting examples of standard treatments for ETEC-associated infection include administration of clear liquids, packaged or premixed oral rehydration salts, chicken soup, bismuth subsalicylate, and antibiotics. Further, a vaccine composition of the disclosure may be administered in combination with other vaccine compositions for ETEC-associated infection. Non-limiting examples of other ETEC vaccine compositions include ACE527, ETVAX, LTR192G with dscCfaE, Vivotif+Dukoral, and ETEC/rCTB.

As used herein, “subject” or “patient” is used interchangeably. Suitable subjects include, but are not limited to, a human, a livestock animal, a companion animal, a lab animal, and a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In specific embodiments, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc. In a preferred embodiment, the subject is human.

A subject may be a subject at risk of ETEC infection. Non-limiting examples of subjects at risk of ETEC infection include subjects residing in an ETEC endemic region, subjects traveling to an ETEC endemic region, subjects residing in a location with a recent outbreak of ETEC, subjects suspected of ingesting ETEC-contaminated food or water. In certain embodiments, a subject may be an infant, toddler or young child.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Introduction to the Examples

The enterotoxigenic Escherichia coli (ETEC) are among the most common causes of infectious diarrhea worldwide. Importantly, ETEC are disproportionately represented in cases of severe diarrheal illness as well as in deaths due to diarrhea among young children in developing countries [1].

These pathogens cause diarrhea by the elaboration and effective delivery of heat-labile and/or heat-stable enterotoxins to intestinal epithelial cells where they stimulate production of cyclic nucleotides ultimately activating the cystic fibrosis transmembrane regulator (CFTR) with resulting net efflux of fluid into the intestinal lumen [2]. Plasmid-encoded colonization factors (CFs), discovered [3] shortly after these organisms were identified as a causative agent of cholera-like diarrheal illness [4-6], are thought to be essential for effective colonization of the small intestine and required for ETEC pathogenesis.

Following early studies suggesting a pivotal role for these structures [7,8], CF antigens have defined the basis for most subsequent ETEC vaccine efforts [9,10]. However, one factor complicating development of a broadly protective vaccine for ETEC has been the general plasticity of E. coli genomes [11], and the significant antigenic heterogeneity of the CFs. To date, at least 26 antigenically distinct CF antigens have been described [12]. The lack of appreciable cross-protection afforded by these antigens combined with the complex landscape of CFs portrayed in ETEC molecular epidemiology studies continue to complicate rational CF antigen selection [13].

Antigenic heterogeneity, recent failure of LT-toxoid-based vaccine strategies [14,15], as well as the need to optimize the performance of live-attenuated vaccines currently in clinical trials [16-18] have highlighted the need to identify additional virulence molecules that might be targeted in ETEC vaccines.

### Example 1. Conservation of ETEC Pathogen-Specific Secreted Antigens

Two antigens, the EtpA adhesin, and the passenger domain of the EatA serine protease are encoded on the large 92 kilobase virulence plasmid of the prototypical ETEC strain H10407. Both of these secreted proteins [22,30] are required for H10407 to efficiently deliver heat-labile toxin to target epithelial cells. To assess their utility as potential vaccine antigens, we examined a large collection of ETEC strains that were well characterized with respect to associated clinical meta-data pertaining to disease severity and which had not undergone repeated serial passage in the laboratory.

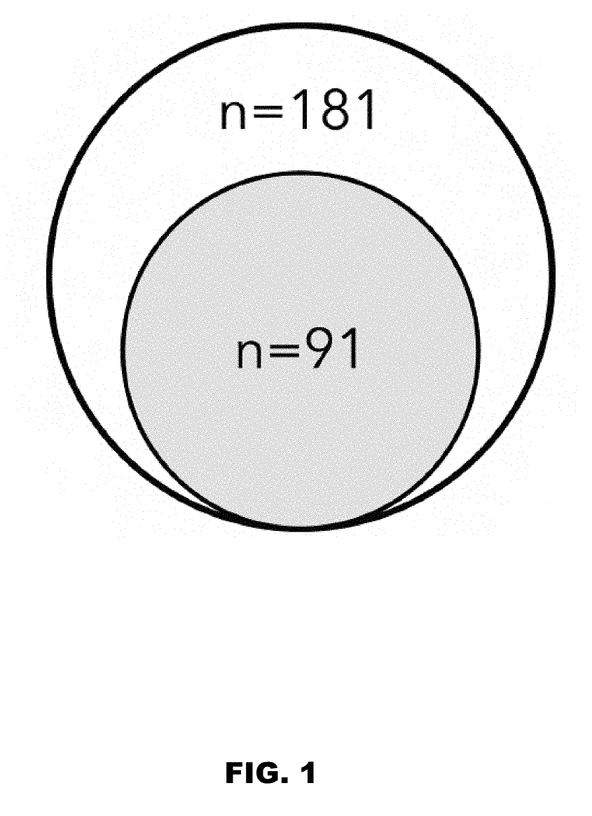

Altogether, we found that these antigens are relatively conserved in the ETEC pathovar. Of the 181 strains examined in the present study (FIG. 1), we found that more than half of all strains produced EtpA (102/181, 56%) and/or EatA (106/181, 59%) (Table 6, Table 7), and that more than three quarters of all strains produced at least one of these antigens. Both EtpA and EatA were identified more than twice as frequently as the most commonly identified CF (CS6), which was identified in 22% of strains in this collection (Table 1).

TABLE 1
Distribution of EtpA and EatA among strains expressing different

| | | | | EtpA n+ or |
|---|---|---|---|---|
| CF¹ | total (n) | EtpA n+ (%)² | EatA n+ (%)² | EatA n+ (%)² |
| CFA/I | 21 | 17 (81) | 17 (81) | 18 (85) |

CFA/II³

| | | | | |
|---|---|---|---|---|
| cs1 | 14 | 12 (86) | 12 (86) | 13 (93) |
| cs2 | 10 | 8 (80) | 7 (70) | 9 (90) |
| cs3 | 25 | 21 (84) | 19 (76) | 23 (92) |

CFA/IV³

| | | | | |
|---|---|---|---|---|
| cs4 | 7 | 1 (14) | 3 (43) | 3 (43) |
| cs5 | 23 | 0 (0) | 18 (78) | 18 (78) |
| cs6 | 39 | 3 (8) | 25 (64) | 26 (67) |

Other CFs

| | | | | |
|---|---|---|---|---|
| cs7 | 16 | 14 (88) | 15 (94) | 15 (94) |
| cs8 | 2 | 0 (0) | 2 (100) | 2 (100) |
| cs14 | 14 | 12 (86) | 2 (14) | 13 (93) |
| cs17 | 10 | 9 (90) | 9 (90) | 9 (90) |
| cs21 | 17 | 14 (82) | 14 (82) | 16 (94) |
| nd | 36 | 16 (44) | 7 (19) | 18 (50) |

¹expression as determined with corresponding monoclonal antibody
²refers to strains expressing proteins as determined by immunoblotting
nd: not detected with any of the above monoclonal antibodies
³(CFA/II pili are typically comprised of CS1 or CS2 in combination with CS3; CFA/IV pili may be formed by CS4 or CS5 in combination with CS6 or CS6 alone.)

### Example 2. Relationship of Plasmid-Encoded Virulence Loci to Colonization Factor Antigens

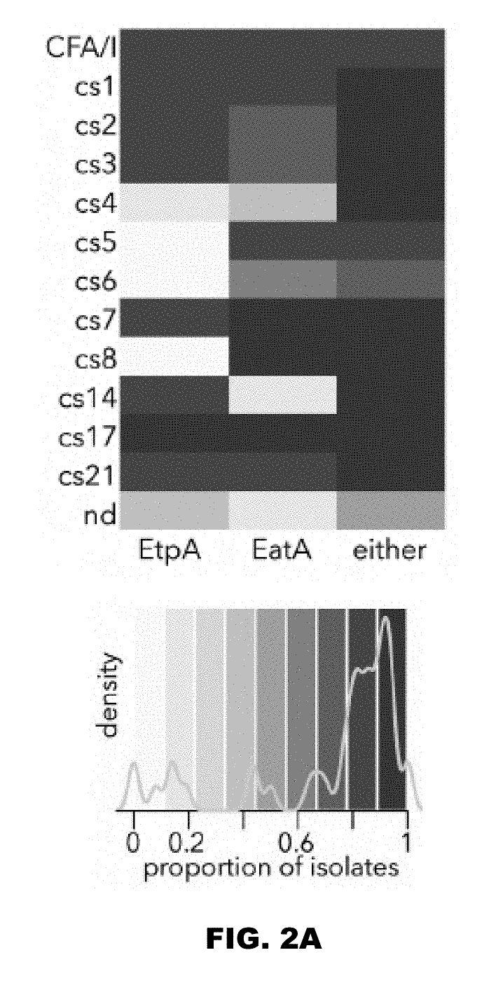

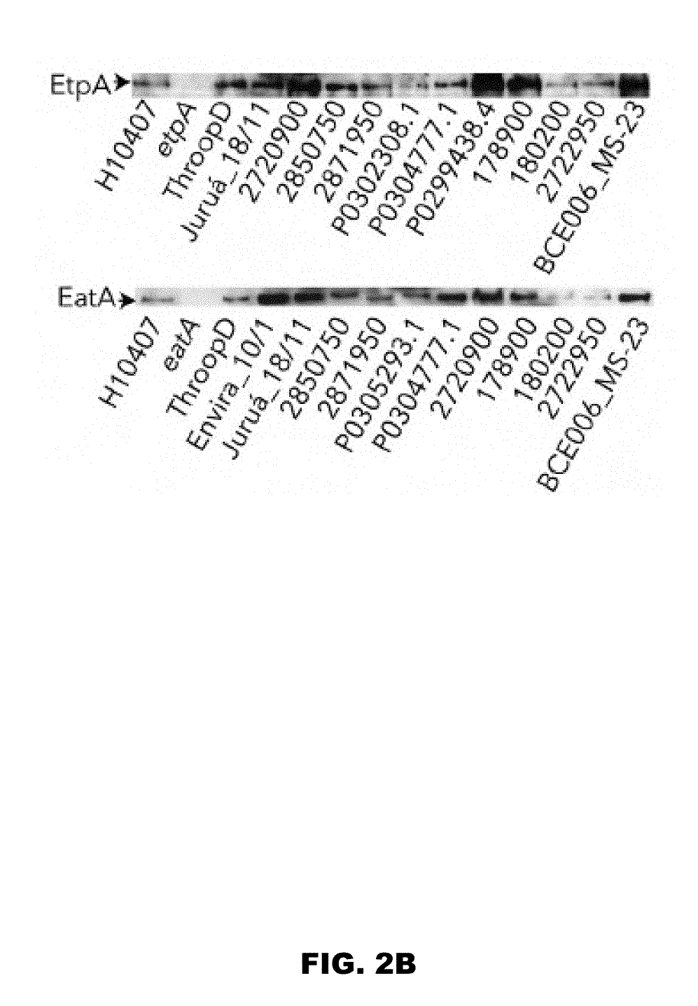

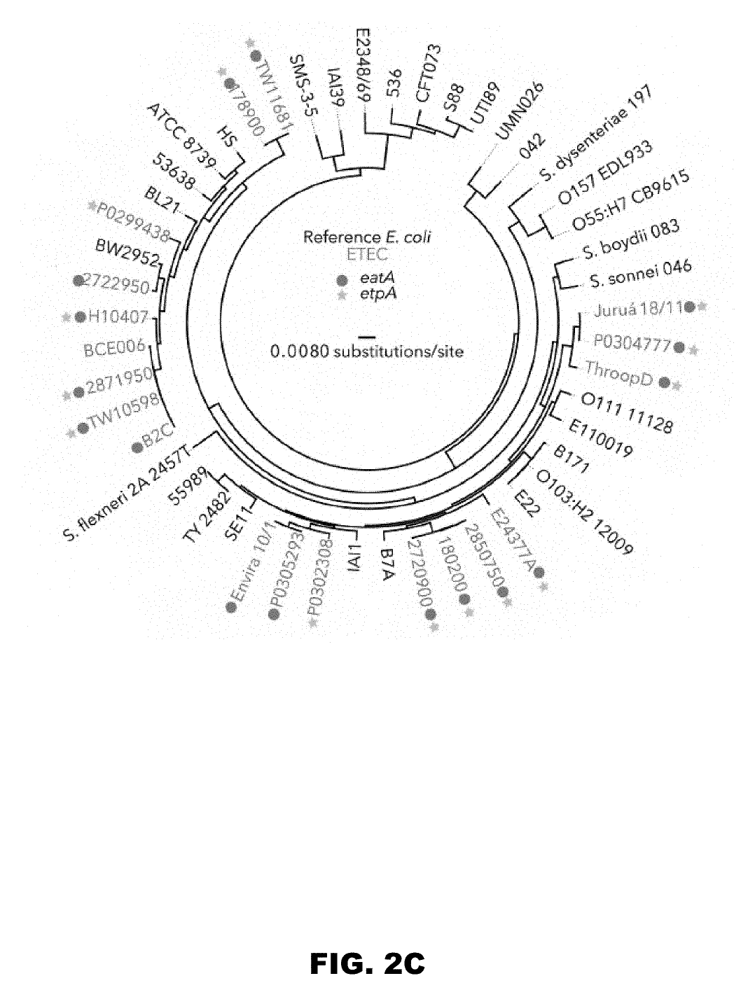

Importantly, although the genes encoding the etpBAC secretion system [19] and the EatA auto-transporter [21] were initially discovered on the same large virulence plasmid of H10407, which also encodes the colonization factor (CF) CFA/I, we found that these loci were not restricted to strains expressing this particular CF, but were widely distributed among the different CFs, and were also present in strains for which no CF could be identified (FIG. 2A). Indeed, half of the strains for which no CF could be identified expressed either EtpA or EatA, suggesting that these antigens could complement existing vaccination strategies centered on CFs. As expected by the association with multiple CFs, we also found that EtpA and EatA were secreted by strains from multiple phylogenic lineages (FIG. 2B, FIG. 2C). Interestingly, however we found a negative association between the etpBAC locus and strains expressing CFA/IV antigens [50,51] including CS5 in that none of the 23 strains possessing CS5 fimbriae secreted the EtpA adhesin. Similarly, among strains expressing CS6, which is frequently co-expressed with CS5, only a minority secreted EtpA.

As has been noted previously, ETEC strain H10407 causes more severe illness in human clinical challenge studies relative to other strains like B7A [53]. Because we had clinical metadata pertaining to disease severity for all of the strains in our collection, we questioned whether the production of either of EatA and/or EtpA was associated with strains isolated from more severe forms of infection. However, we did not find any clear association between either of these putative virulence loci and clinical outcome (Table 6, Table 7).

### Example 3. Conservation of Chromosomally-Encoded Antigens

We also examined the conservation of two chromosomally-encoded antigens which are not specific to the ETEC pathovar. The eaeH gene was originally identified on the chromosome of ETEC strain H10407 by subtractive hybridization with E. coli MG1655 [54], is transcriptionally activated by cell contact [26], and under these conditions EaeH is produced by a diverse group of strains belonging to different phylogenies [28]. Using the EaeH peptide sequence from H10407 (GenBank accession AAZ57201), BLASTP searches of recently sequenced ETEC strains from Bangladesh and elsewhere (gscid.igs.umaryland.edu/wp.php?wp=comparative_genom e_analysis_of_enterotoxigenic_e._coli_isolates_from_infections_of_different_clinical_severity) also revealed that the eaeH gene was present in 63 out of 91 distinct isolates (69%) (Table 9). BLASTP searches of these data for another chromosomally encoded molecule, YghJ, a type II secretion system effector [55] recently shown to be involved in mucin degradation and toxin delivery [27] demonstrated that the yghJ gene was present on the chromosomes in 83 of 91 (91%) isolates. Similarly, we identified the YghJ protein in a majority (161/181, 89%) of ETEC culture supernatants (Table 8). This antigen was produced across ETEC strains expressing multiple CF types including 31/36 strains that were CF-negative by monoclonal antibody screening.

### Example 4. EatA and EtpA Sequence Conservation

Ideally, putative vaccine targets should be specific to the pathovar under study or restricted to pathogenic isolates, but not subject to significant antigenic variation. Therefore to further examine the potential utility of two ETEC pathovar specific antigens, EtpA and EatA, as vaccine candidates, we used recently obtained DNA sequence information from multiple ETEC genomes belonging to different phylogenies and from temporally and geographically disparate sources to compare the predicted amino acid sequences of these proteins.

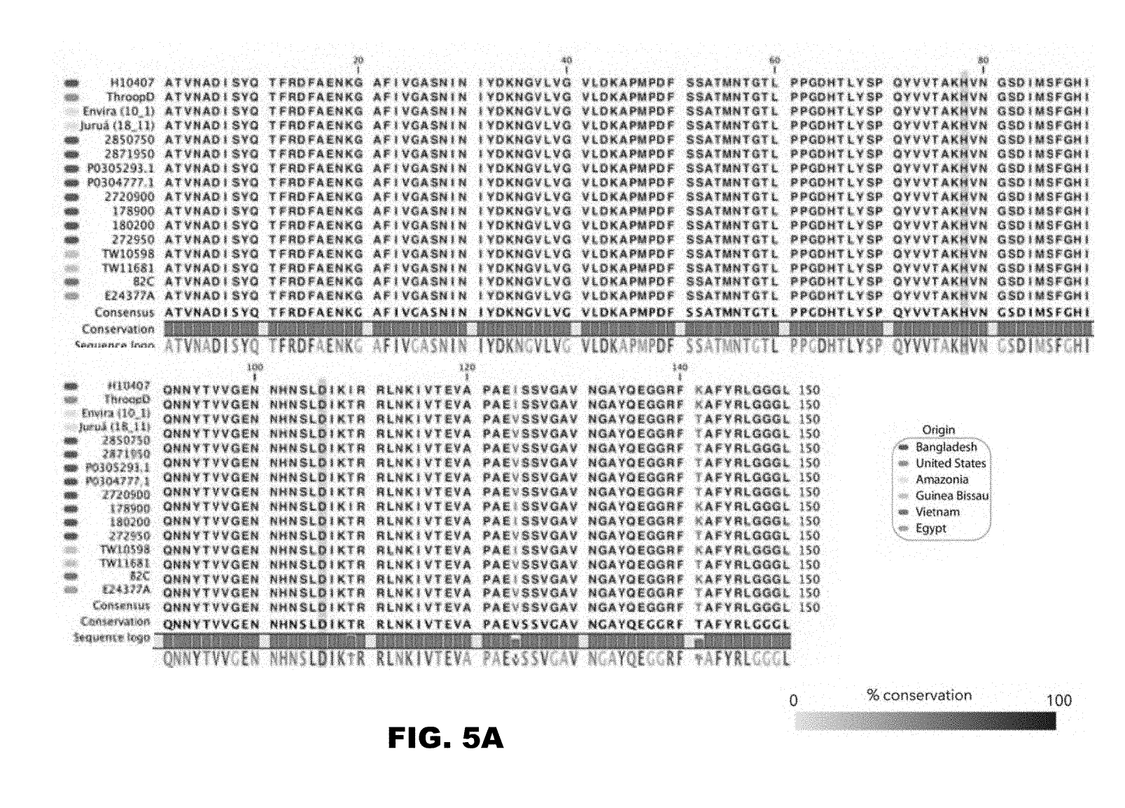

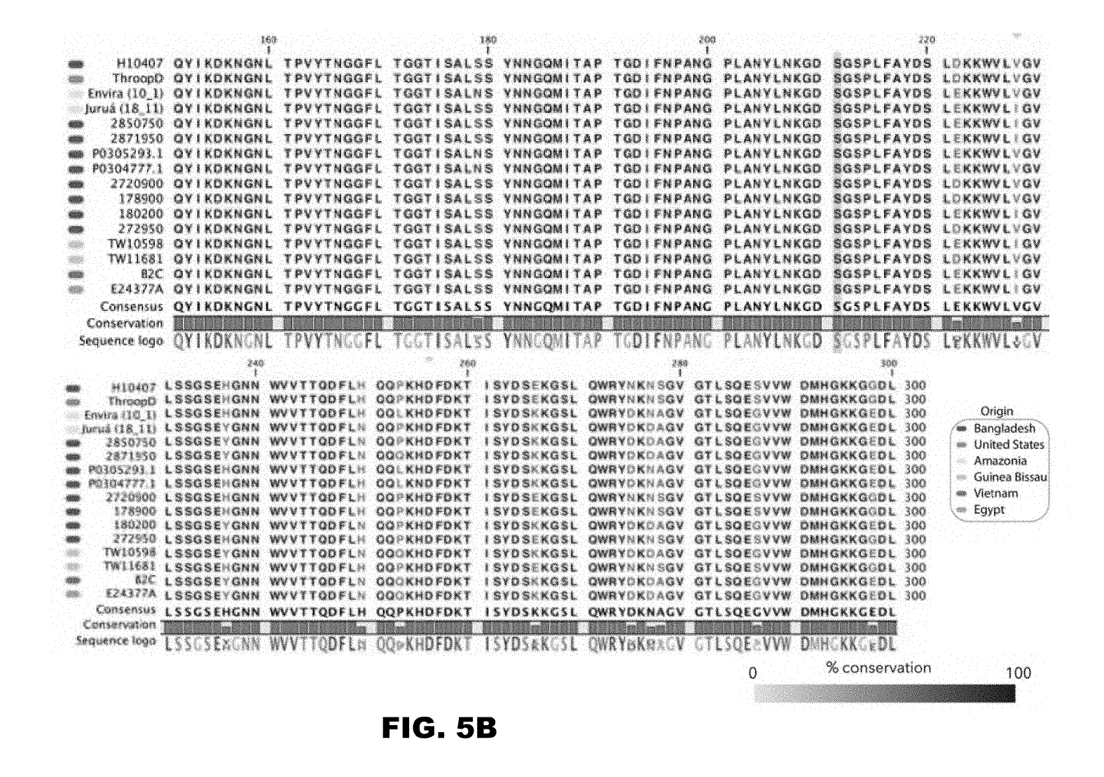

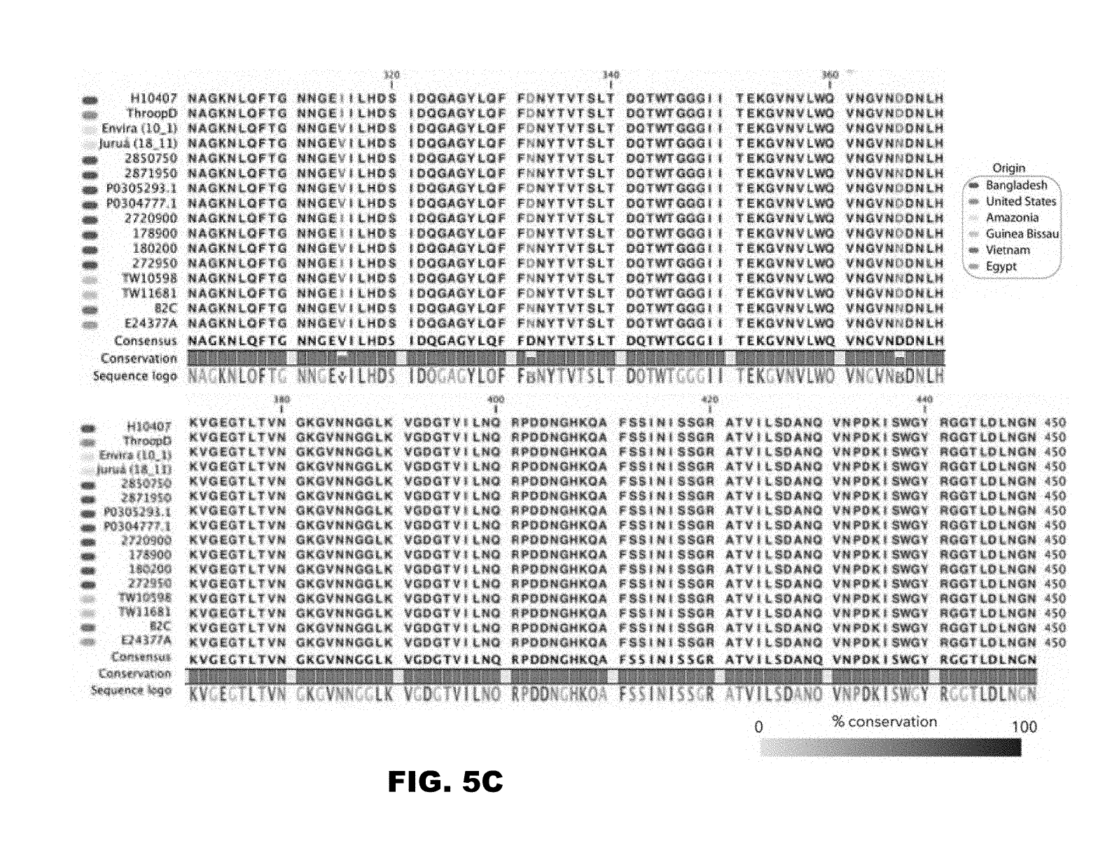

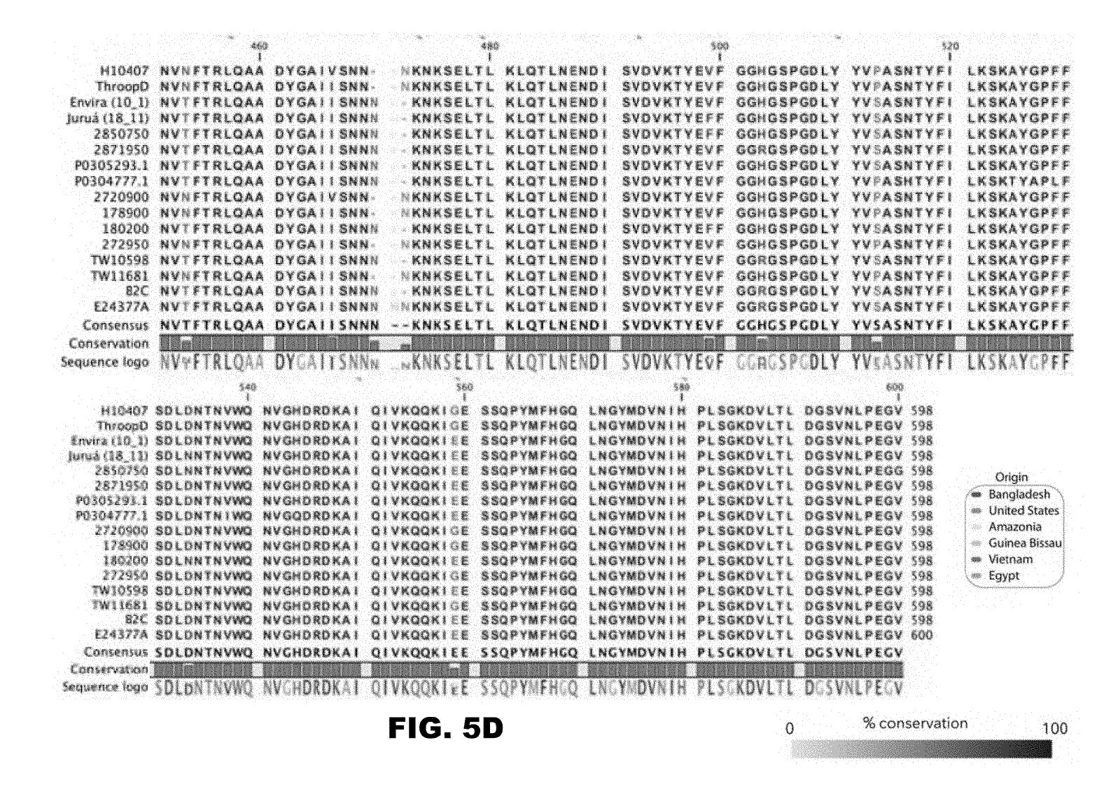

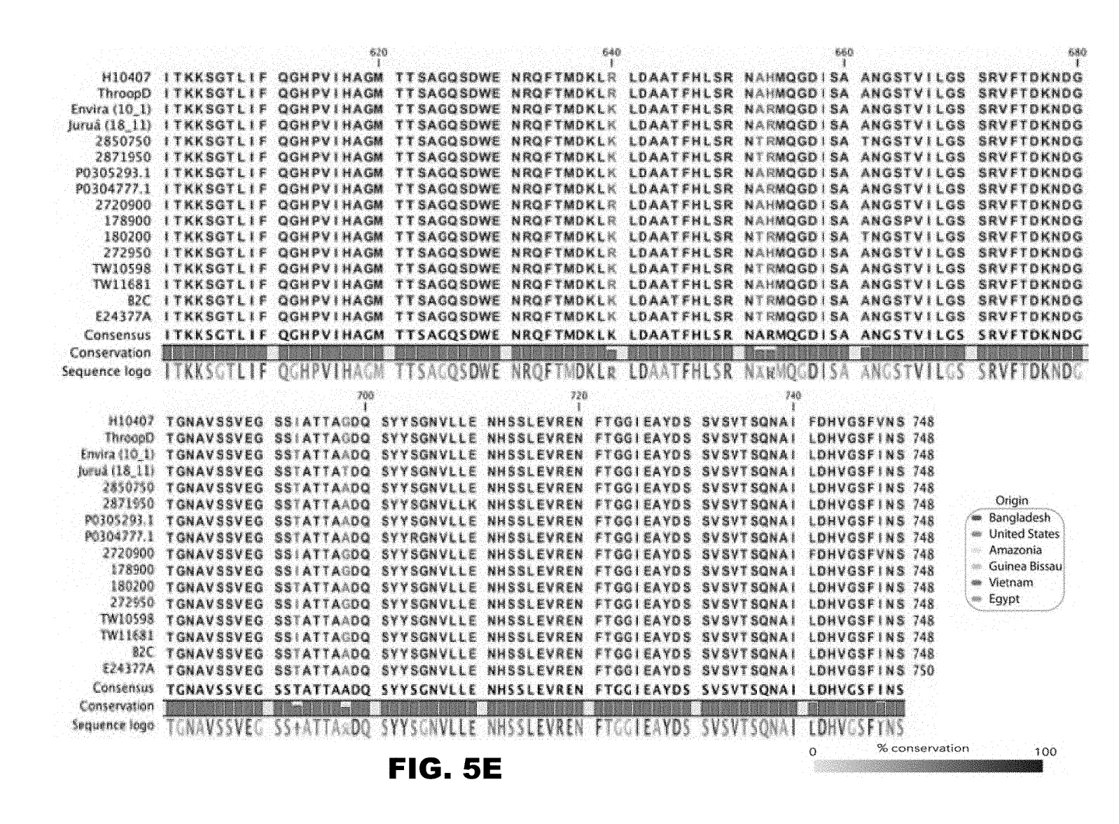

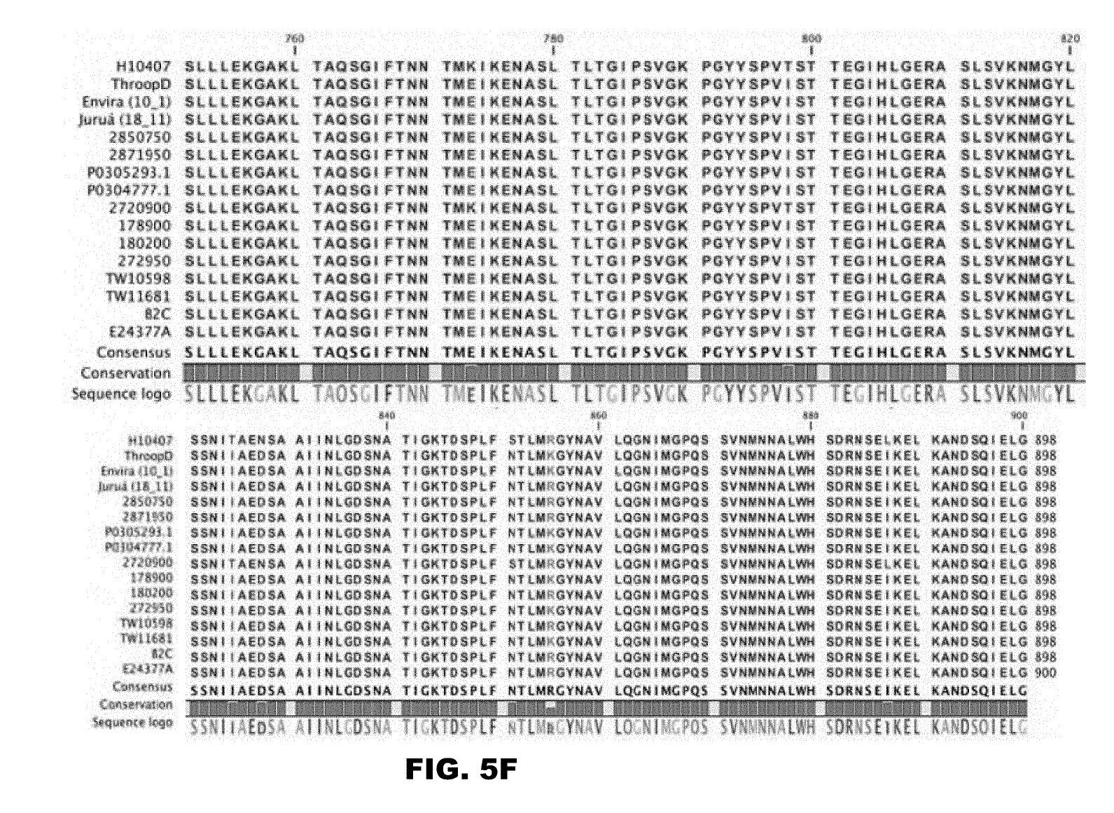

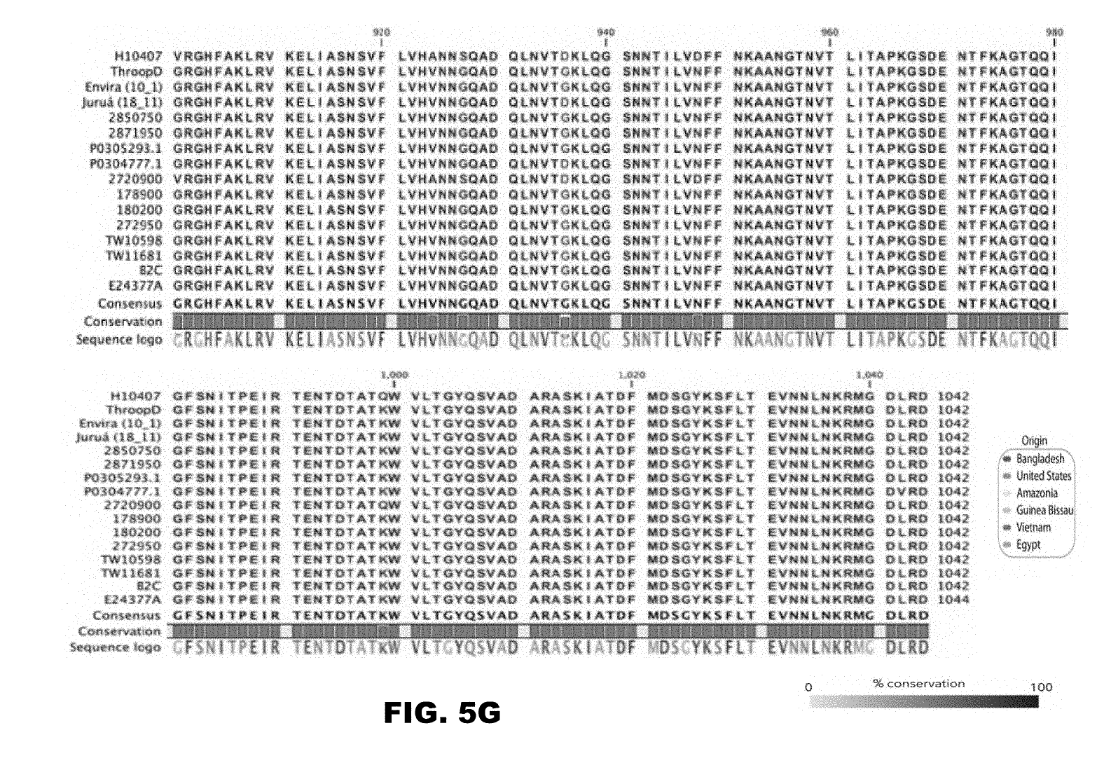

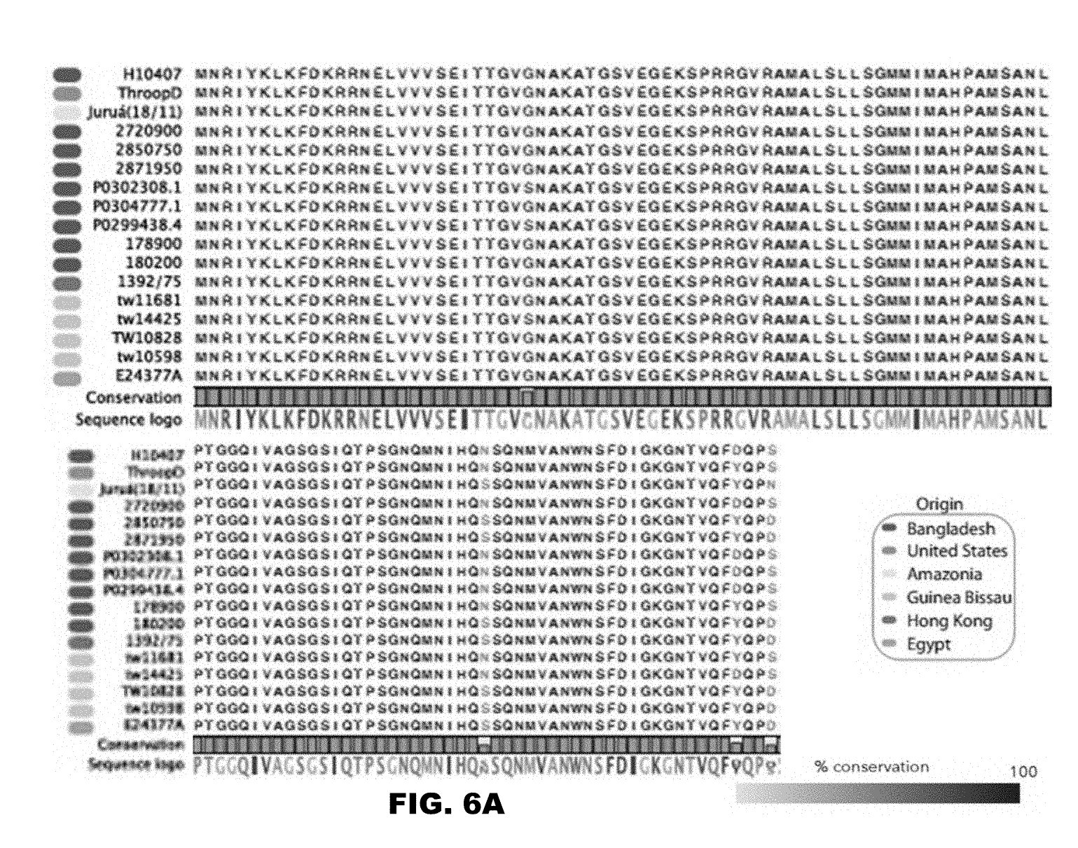

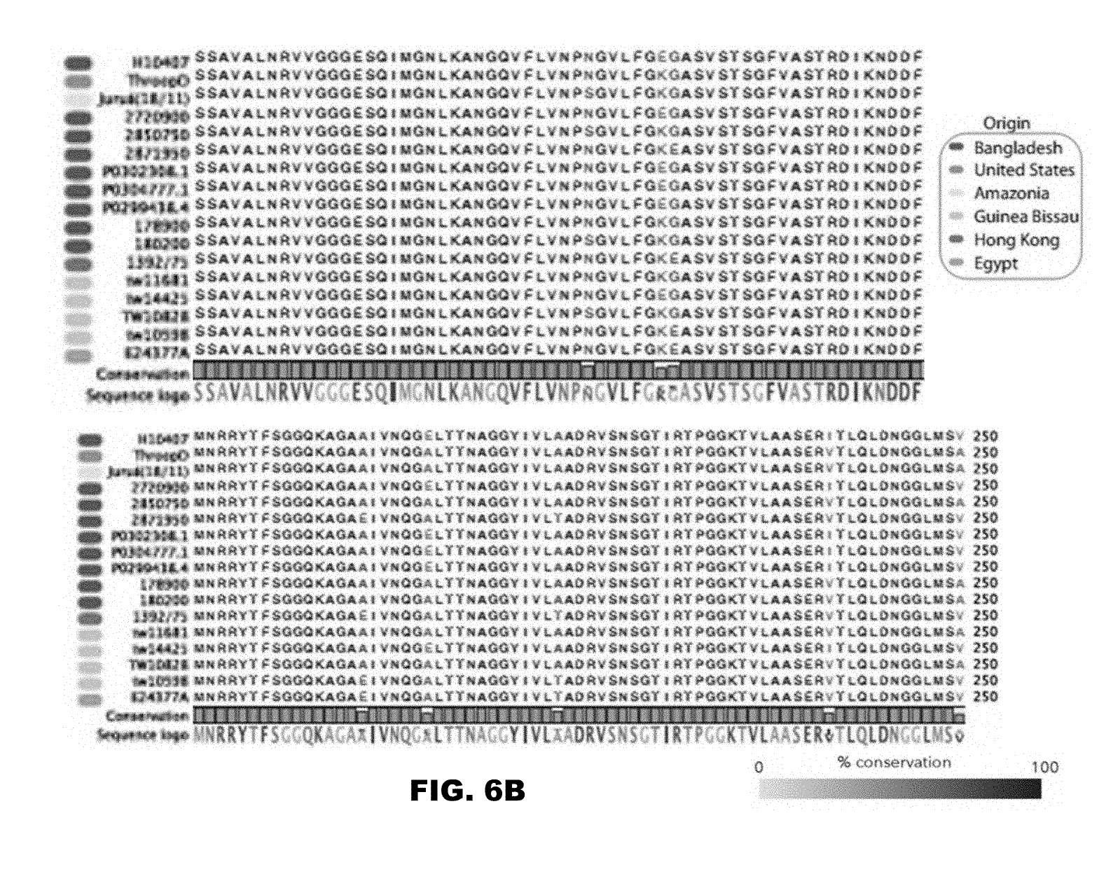

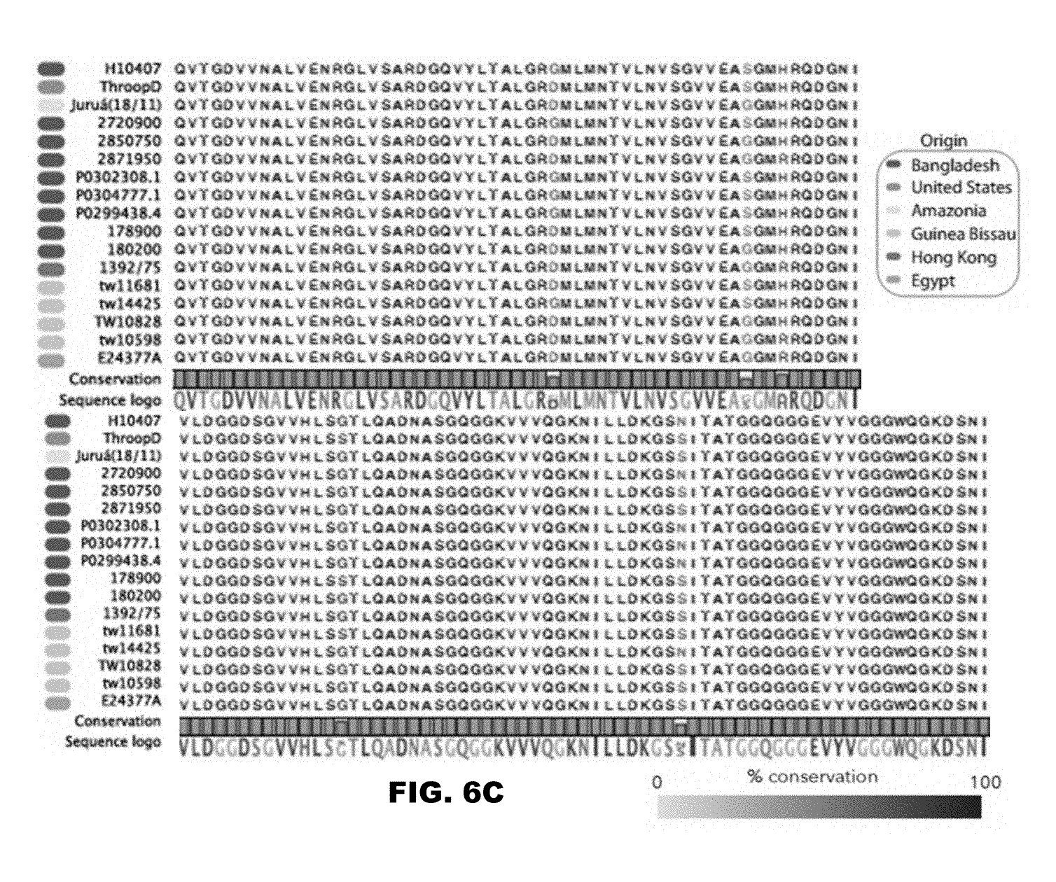

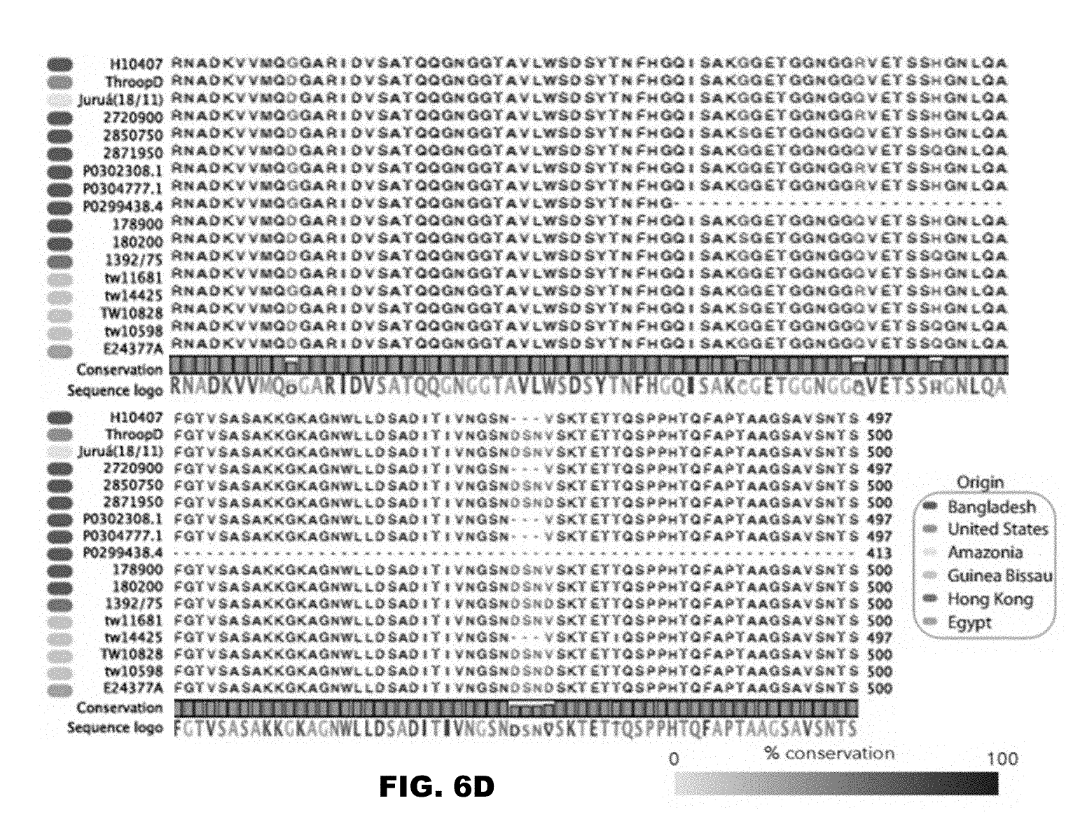

For the prototype EatA molecule, first described in ETEC H10407 [21], the 1042 residue region from amino acids 57-1098 is predicted for the secreted passenger domain that contains the serine protease catalytic triad [21] as well as protective epitopes [23]. We therefore compared this region of the molecule to those derived from the recently released genome sequences of multiple ETEC strains. Altogether, we found that the sequence of the EatA passenger domain (EatAₚ) was very highly conserved across strains, and exhibited between 95-100% identity to the prototype H10407 EatA (Table 2). Likewise, the predicted serine protease catalytic motif formed by the histidine, aspartic acid and serine residues at positions 134,162, and 267, respectively were universally conserved within the passenger domains of these proteins (FIG. 5). Similarly, the predicted amino acid sequences of the secreted EtpA adhesin molecules from multiple strains exhibited between 94 and 100% identity to the H10407 prototype antigen (Table 3, FIG. 6).

Despite the fact that the comparator strains included here spanned isolates collected over nearly 40 years, belonging to different phylogenies and that strains originated in diverse locations in Asia, Africa and the Americas, both proteins appear to exhibit remarkably little antigenic variation. Likewise, in analysis of the genomes of strains isolated recently within Bangladesh both proteins demonstrated similar degrees of sequence conservation (FIG. 5, FIG. 6).

TABLE 2
EatA sequence conservation in geographically, temporally, and phylogenically
disparate isolates.

| | | | isolation | | identity¹ | similarity | |
|---|---|---|---|---|---|---|---|
| strain | accession | origin | reported | phenotype | (%) | (%) | ref. |
| H10407 | Q84GK0.1 | Bangladesh | 1973 | cholera-like | — | — | [52] |
| Throop D | ²EMW91712.1 | Dallas, TX | 1976 | cholera-like | 98 | 99 | [36] |
| Envira 10/1 | ²EMX71514.1 | Amazon, Brazil | 1998 | cholera-like | 97 | 98 | [35] |
| Jurua 18/11 | ²EMX58104.1 | Amazon, Brazil | 1998 | cholera-like | 96 | 98 | [35] |
| 2850750 | ²EMW01116.1 | Bangladesh | 2008 | cholera-like | 96 | 97 | ⁵ |
| 2871950 | ²EMV49671.1 | Bangladesh | 2008 | cholera-like | 96 | 98 | ⁵ |
| P0305293.1 | ²EMZ82436.1 | Bangladesh | 2011 | cholera-like | 97 | 98 | ⁵ |
| P0304777.1 | ²EMX02137.1 | Bangladesh | 2011 | cholera-like | 96 | 97 | ⁵ |
| 2720900 | ²EMX91421.1 | Bangladesh | 2007 | cholera-like | 100 | 100 | ⁵ |
| 178900 | ²ENA71292.1 | Bangladesh | 2010 | cholera-like | 98 | 99 | ⁵ |
| 180200 | ²ENA61350.1 | Bangladesh | 2010 | cholera-like | 96 | 98 | ⁵ |
| 2722950 | ²EMZ73955.1 | Bangladesh | 2007 | mild-self- | 98 | 99 | ⁵ |
| | | | | limited | | | |
| tw10598 | ³AELA00000000.1 | Guinea-Bissau | 1996 | diarrhea⁴ | 96 | 98 | [49] |
| tw10722 | ³AELB00000000.1 | Guinea-Bissau | 1996 | diarrhea⁴ | 95 | 98 | [49] |
| tw10828 | ³AELC00000000.1 | Guinea-Bissau | 1996 | diarrhea⁴ | 95 | 97 | [49] |
| tw11681 | ³AELD00000000.1 | Guinea-Bissau | 1996 | diarrhea⁴ | 98 | 99 | [49] |
| B2C | ETS27975.1 | Vietnam | 1971 | diarrhea⁴ | 96 | 98 | [62] |
| E24377A | YP_01451588.1 | Egypt | 1980s | diarrhea⁴ | 96 | 97 | [63] |

¹based on BLAST-P searches against 1042 residues of predicted passenger domain of H10407.
²sequenced at GSCID (gscid.igs.umaryland.edu/wp.php?wp=comparative_genome_analysis_of_enterotoxigenic_e._coli_isolates_from_infections_of_different_clinical_severity)
³open reading frames corresponding to the eatA gene were first assembled from whole genome shotgun sequence contigs for these draft genomes; BLASTP for these homologues was conducted using CLC Main Workbench v6.9.1 and local database of predicted protein sequences derived from translation of assembled contigs from the respective sequencing projects.
⁴(severity unknown)
⁵this study

TABLE 3
EtpA sequence conservation in geographically, temporally, and phylogenically
disparate isolates.

| | | | isolation | | identity¹ | similarity | |
|---|---|---|---|---|---|---|---|
| strain | accession | origin | reported | phenotype | (%) | (%) | ref. |
| H10407 | AAX13509.2 | Bangladesh | 1973 | cholera-like | — | — | [52] |
| Throop D | ²EMW91721.1 | Dallas, TX | 1976 | cholera-like | 97 | 97 | [36] |
| Jurua 18/11 | ²EMX66921.1 | Amazon, Brazil | 1998 | cholera-like | 94 | 95 | [35] |
| 2720900 | ²EMX81000.1 | Bangladesh | 2007 | cholera-like | 100 | 100 | ⁵ |
| 2850750 | ²EMW11804.1 | Bangladesh | 2008 | cholera-like | 94 | 95 | ⁵ |
| 2871950 | ²EMV50451.1 | Bangladesh | 2011 | cholera-like | 95 | 95 | ⁵ |
| p0302308.1 | ²EMX03881.1 | Bangladesh | 2011 | cholera-like | 98 | 98 | ⁵ |
| p0304777.1 | ²EMX02126.1 | Bangladesh | 2011 | cholera-like | 99 | 98 | ⁵ |
| p0299438.4 | ²ENB94816.1 | Bangladesh | 2011 | cholera-like | 99 | 99 | ⁵ |
| 178900 | ²ENA69446.1 | Bangladesh | 2010 | cholera-like | 97 | 97 | ⁵ |
| 180200 | ²ENA71844.1 | Bangladesh | 2010 | cholera-like | 94 | 95 | ⁵ |
| 1392/75 | YP_003717617.1 | Hong Kong | — | diarrhea⁴ | 95 | 96 | [61] |
| tw11681 | ³AELD00000000.1 | Guinea-Bissau | 1996 | diarrhea⁴ | 96 | 97 | [49] |
| tw14425 | ³AELE00000000.1 | Guinea-Bissau | 1996 | diarrhea⁴ | 99 | 100 | [49] |
| tw10828 | ³AELC00000000.1 | Guinea-Bissau | 1996 | diarrhea⁴ | 94 | 96 | [49] |
| tw10598 | ³AELA00000000.1 | Guinea-Bissau | 1996 | diarrhea⁴ | 95 | 96 | [49] |
| E24377A | ³NC_009786.1 | Egypt | 1980s | diarrhea⁴ | 95 | 96 | [63] |

¹identity and similarity percentages reflect BLASTP comparisons of the first 600 residues of the EtpA sequence.
²sequenced at GSCID (gscid.igs.umaryland.edu/wp.php?wp=comparative_genome_analysis_of_enterotoxigenic_e._coli_isolates_from_infections_of_different_clinical_severity)
³open reading frames corresponding to the etpA gene were first assembled from whole genome shotgun sequence contigs for these draft genomes; BLASTP for these homologues was conducted using CLC Main Workbench v6.9.1 and local database of predicted protein sequences derived from translation of assembled contigs from the respective sequencing projects.
⁴(severity unknown)
⁵this study

### Example 5. Immunogenicity of Novel Virulence Antigens

Earlier immunoproteomic studies suggested that a variety of conserved E. coli proteins as well as ETEC pathovar specific proteins are recognized during the course of experimental infections in mice, and these responses parallel those observed using pooled convalescent sera from ETEC patients [25]. To further characterize the immune response to novel antigens, we focused on four, including two plasmid-encoded secreted ETEC pathovar-specific antigens: EatA protease, and the EtpA adhesin, as well as the highly conserved chromosomally-encoded YghJ metalloprotease and the EaeH adhesin protein.

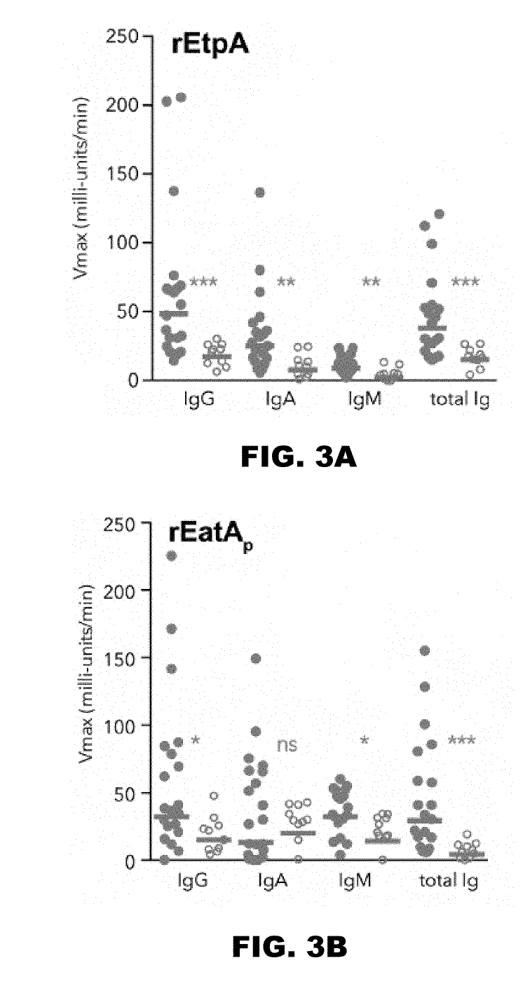

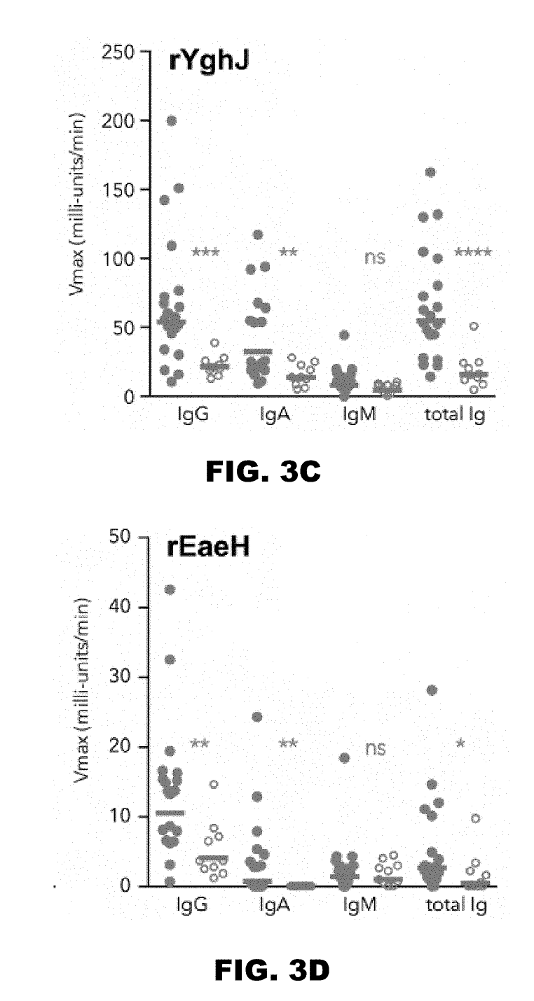

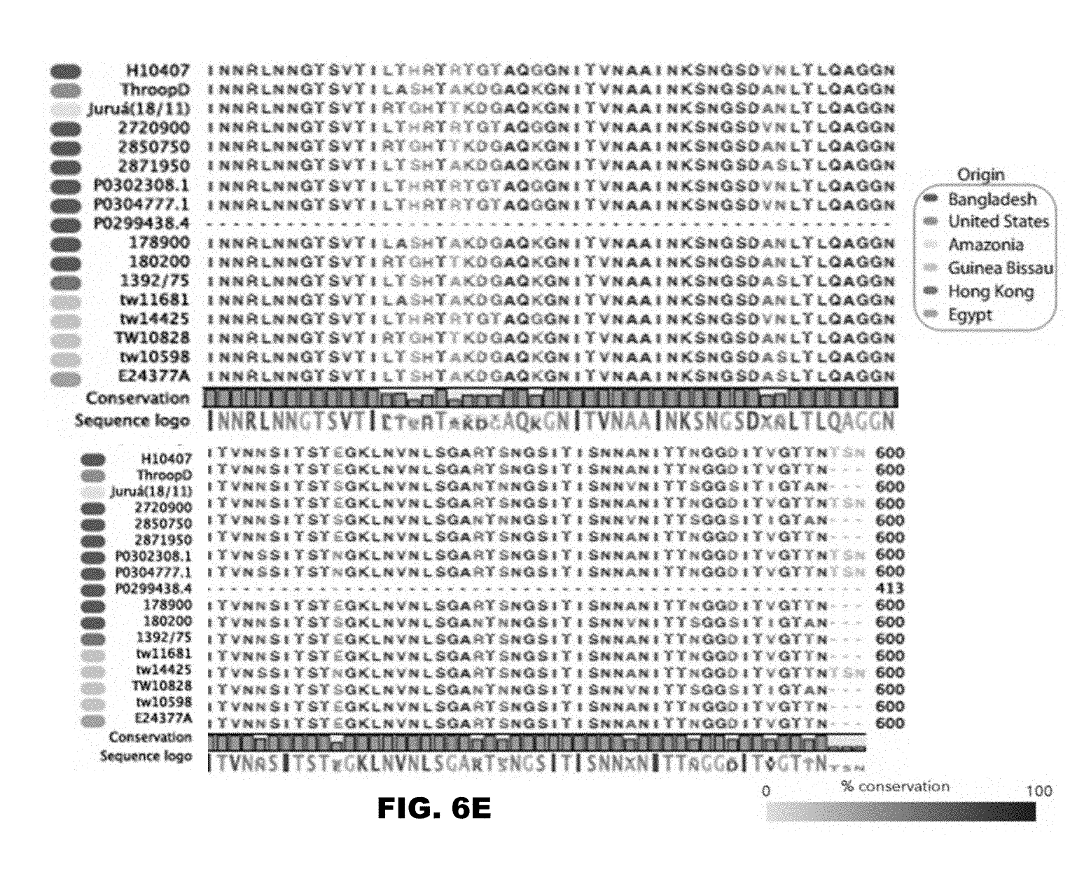

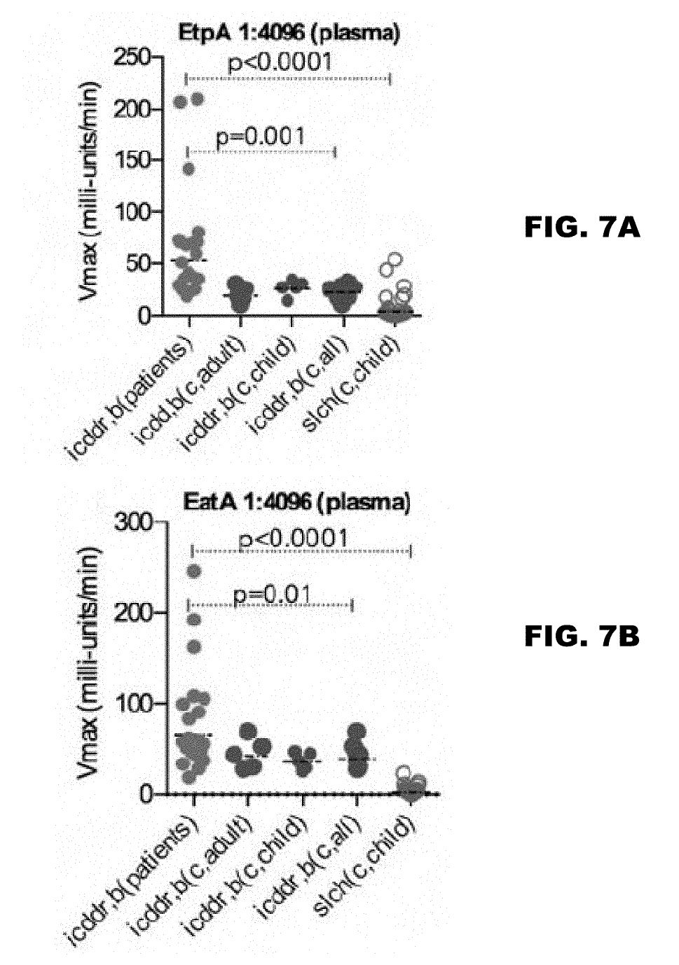

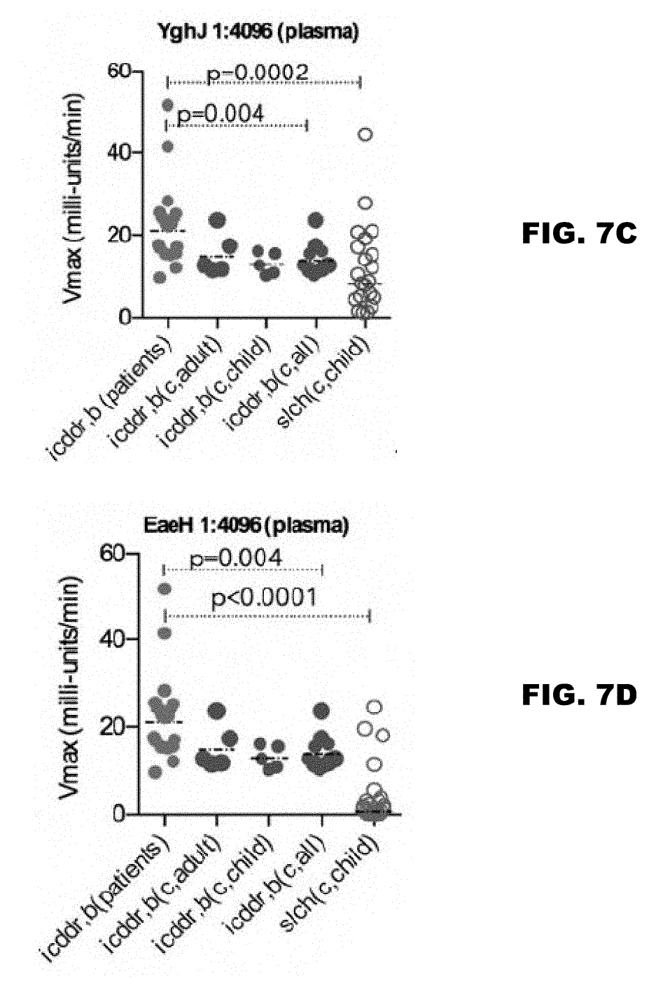

In comparing convalescent plasma from patients hospitalized at icddr,b to uninfected controls from Bangladesh, we found that patients in general exhibited significantly greater total antibody (IgG, IgM, IgA) responses to each of these antigens following diarrheal illness (FIG. 3) suggesting that these proteins are expressed during the course of infection. Similar results were obtained in comparing plasma from un-infected children from a non-endemic area in the United States (FIG. 7).

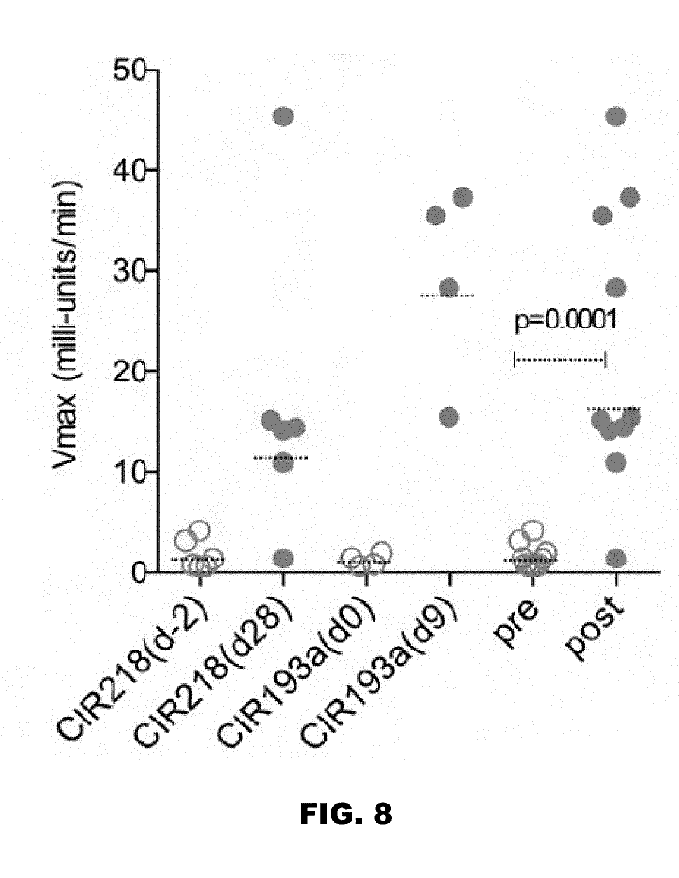

We also examined the immune response to EtpA following infection by examining sera obtained before and after challenge of human volunteers with ETEC H10407. In sera obtained from two independent volunteer challenge studies, we also observed significant increases in immune responses to EtpA (FIG. 8), strongly suggesting that this secreted protein is specifically recognized following infection by ETEC strains including H10407 that secrete this antigen.

### Example 6. Protective Efficacy of Combined EtpA-Mutant EatA Passenger Vaccination

The data above suggest that collectively these antigens might significantly extend coverage presently offered by classical approaches to ETEC vaccine development. We therefore questioned whether these two antigens could be successfully combined in a subunit approach. Because we have previously demonstrated that the native secreted EatA passenger domain will degrade intestinal mucin [29] as well as the EtpA adhesin molecule [22], we elected to vaccinate animals with a modified recombinant version of the EatA passenger that lacks protease activity (rEatAₚH134R).

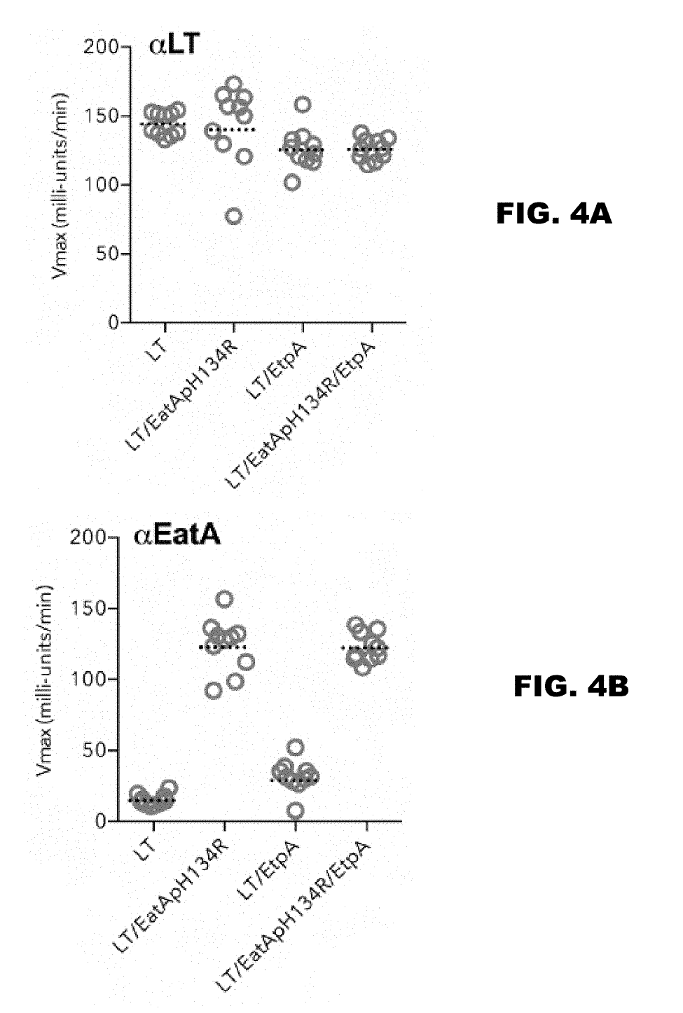

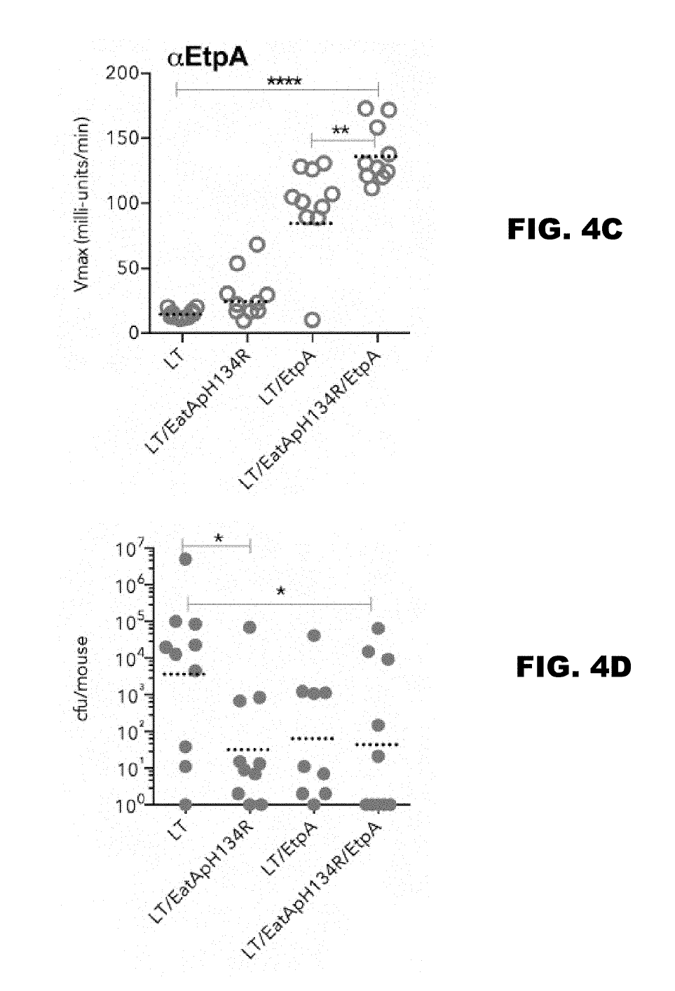

Co-vaccination with rEtpA and the mutant rEatAₚH134R molecule elicited robust serologic responses to both molecules. As anticipated, each of the groups mounted strong serologic responses to the LT adjuvant (FIG. 4A), and both antigens retained their immunogenicity following co-immunization of EtpA with the rEatAH134R passenger domain (FIG. 4B, FIG. 4C) with responses that were at least comparable to those obtained following immunization with either antigen alone (FIG. 4C). Likewise, mice immunized with both antigens were significantly protected against colonization by ETEC (FIG. 4D). Collectively, these data suggest that co-immunization with these two antigens is feasible, and could be employed to expand present approaches to ETEC vaccine antigen selection.

### Discussion for the Examples

Enterotoxigenic Escherichia coli remain one of the most common causes of infectious diarrhea worldwide, and severe disease caused by these pathogens persists as leading cause of death among young children in developing countries [1]. Despite recognition of these toxin producing E. coli as a cause of severe cholera-like diarrheal illness more than forty years ago [57], there remains no effective broadly protective vaccine for ETEC.

Most vaccinology efforts to date have focused almost exclusively on a subset of plasmid-encoded antigens, namely the colonization factors (CFs) and heat-labile toxin [9]. Vaccines based on this strategy have faced several impediments. First, the CFs are quite diverse with more than 26 distinct antigens described to date. In addition, a number of recent vaccine studies have suggested that simply engendering immune responses to CFs and/or heat-labile toxin may not be sufficient to provide sustained broad-based protection [14-16].

A major challenge to ETEC vaccine development in general is that the most highly conserved antigens of ETEC, typically encoded on core regions of the chromosome, are also shared with commensal E. coli [60]. Included among these chromosomally encoded conserved proteins are two antigens studied here, YghJ [27] and EaeH [28] that were recently shown to be important for ETEC virulence. While the present studies also demonstrate that these proteins are recognized during the course of ETEC infection, the degree to which these antigens can be safely targeted in vaccines without inadvertent disruption of the intestinal microflora remains to be studied.

The inherent plasticity of E. coli genomes contributes substantially to the difficulty in defining antigens unique to the ETEC pathovar that are widely conserved. No single antigen exclusive to these pathogens, but universally conserved in this pathovar, has been described to date. Some have suggested that this might be predicted based on the fact that the plasmid-encoded heat-labile and/or heat-stable toxins, which define the ETEC pathovar, could form a minimal complement of virulence genes in wide variety of E. coli host strains [61].

In this context, we examined the gene conservation and the actual production of these proteins in a large collection of well-characterized strains from Bangladesh, complemented by strains from other locations that were associated with severe disease and for which there were available clinical metadata. Notably, two plasmid-encoded ETEC pathotype-specific antigens, the EatA serine protease and the secreted EtpA adhesin molecule were shared broadly among strains belonging to different CF groups with the exception of strains that produced CFA/IV antigens CS4, CS5, CS6 which only infrequently produced EtpA.

In general, we found high degrees of concordance between the presence of these genes by PCR and production of the corresponding protein. The prevalence of EtpA and EatA was 56 and 59%, respectively, as determined by examination of protein expression. Importantly, the strains that produced these antigens belonged to many different phylogenies suggesting that genes encoding these antigens have been widely dispersed.

The analyses of strains in this study largely focused on isolates from Bangladesh. However, these data are potentially relevant for vaccine development for a number of reasons. First, Bangladesh is highly endemic for enterotoxigenic E. coli infections, and consequently remains an important site for vaccine field trials. In addition, ETEC has been under study in this region since the discovery of this pathotype, permitting us to compare sequence variation in candidate antigens over four decades. Understanding both current prevalence and sequence conservation of potential novel vaccine antigens in this population over time will be particularly important for making rational decisions about their inclusion in future iterations of ETEC vaccines. Finally, the geographic and temporal dispersal of genes encoding EtpA and EatA in multiple phylogenetic backgrounds, further attests to importance of studying these molecules as vaccine targets.

The data presented here suggest that the novel pathovar-specific antigens could complement existing strategies for ETEC vaccine development by broadening the antigenic valency.

### Methods for the Examples

Bacterial Strains and Growth Conditions:

ETEC strains used in this study are detailed in Table 6, Table 8, Table 9. All strains were grown at 37° in Casamino acids yeast extract media [32] (CAYE: 2.0% Casamino Acids, 0.15% yeast extract, 0.25% NaCl, 0.871% K₂HPO₄, 0.25% glucose, and 0.1% (v/v) trace salts solution consisting of 5% MgSO₄, 0.5% MnCl₂, 0.5% FeCl₃) from frozen glycerol stocks maintained at −80° C.

Strain Characterization by Disease Severity and Colonization Factor Type:

Strains from the International Centre for Diarrhoeal Disease Research (icddr,b) in Dhaka were selected based on their associated disease severity using modified WHO guidelines as previously outlined [33]. Expression of individual CFs was determined by dot immunoblotting with monoclonal antibodies specific to each respective CFs (CF-MAb) as previously described [34]. Briefly, 2 μl of a PBS suspension containing 106 colony forming units of each ETEC strain was dotted onto nitrocellulose, air-dried, blocked with BSA in PBS, followed by detection with CF-MAbs and goat anti-mouse IgG_HRP conjugate. Bound MAbs were then detected with 4-chloro-1-naphthol chromogen and H₂O₂.

Screening for ETEC Virulence Genes by PCR:

We screened a total of 181 ETEC available isolates currently maintained as frozen glycerol stocks in our laboratories. The majority of these strains were collected between 1998 and 2011 in Bangladesh, and were obtained from the icddr,b in Dhaka. Complementing this collection were geographically disparate strains associated with severe diarrheal illness including strains from the Amazon region in Brazil [35], and ThroopD, an isolate from a patient with severe ETEC diarrheal illness who presented in Dallas in the 1970s [36]. Strains encoding eatA and etpA were identified by PCR using primers directed against conserved regions of these genes as previous described [37]. Briefly, a small amount of frozen glycerol stock from each strain was introduced with a sterile pipette tip into a PCR mixture containing the respective primers and a master mix. Toxin genotypes were confirmed in these isolates using multiplex PCR screening for genes encoding heat-labile (LT), and heat-stable toxins (STp, and STh) as previously described [34]. Primer sequences are listed in Table 4.

TABLE 4
Oligonucleotide primers used in this study.

| SEQ | | | | Product | |
|---|---|---|---|---|---|
| ID | | Primer | | size | |
| NO: | Gene | designation | Sequence (5′>3′) | (bp) | Ref |
| 6 | eatA | jf082213.1-F | ATGTGCTTTGGCAGGTTAAT | 1943 | [37] |
| 7 | | jf082213.2-R | ATATCCAGTCAGCACCCACT | | |
| 8 | etpA | jf082213.3-F | GGTTCAGGCAGTATCCAGAC | 999 | [37] |
| 9 | | jf082213.4-R | GGTGTAGCTGTCTGACCACA | | |
| 10 | eltB | jf092313.3-F | ACGGCGTTACTATCCTCTC | 273 | [34] |
| 11 | | jf092313.4-R | TGGTCTCGGTCAGATATGTG | | |
| 12 | est1A | jf092313.5-F | TCTTTCCCCTCTTTTAGTCAG | 166 | [34] |
| 13 | | jf092313.6-R | ACAGGCAGGATTACAACAAAG | | |
| 14 | est1B | jf092313.8-F | AGTGGTCCTGAAAGCATG | 64 | [34] |
| 15 | | jf092313.7-R | TACAAGCAGGATTACAACAC | | |

Immunoblotting for Secreted ETEC Virulence Antigens:

To determine production of secreted virulence antigens by different ETEC strains, supernatants from overnight cultures were first precipitated with trichloroacetic acid (TCA) [19] and resuspended in sample buffer before polyacrylamide gel electrophoresis. Western blotting was then performed using polyclonal rabbit antisera against recombinant versions of either EatA [21], EtpA [19], or YghJ [27] that were pre-absorbed against an E. coli lysate column (Pierce) and affinity-purified using the antigen immobilized on nitrocellulose membranes as previously described [31,38], followed by detection with affinity-purified secondary goat anti-rabbit-(IgG)-HRP conjugate (Santa Cruz Biotechnology, SC2004).

Protein Sequence Comparisons of ETEC Pathovar Specific Antigens:

To examine antigenic conservation of EatA among ETEC isolates for which genomic DNA sequences are currently available, BLASTP [39] was used to search GenBank www.ncbi.nlm.nih.gov/genbank/ using the full length sequence of the EatA protein from strain H10407 (www.ncbi.nlm.nih.gov/protein/AAO17297.1) as the query sequence. To construct alignments of EatA from positive strains, the 1042 residue passenger domain (corresponding to amino acids 57-1098 of EatA from H10407) was compared with EatA of ETEC isolates derived from different phylogenic lineages using a CLUSTAL Omega (release 1.2.0 AndreaGia-como) [40] algorithm plugin for CLC Main Workbench v6.9.1. A similar approach was used to compare the amino-terminal sequence of EtpA (amino acids 1-600, GenBank accession number AAX13509.2).

Conservation Heat Mapping:

Virulence protein expression data from the collection of 181 strains under study were included in the analysis. Heat maps were configured using R [41] version 3.1.0 (2014, www.R-project.org/) using gplots [42] and RColorBrewer [43] packages installed from CRAN.R-project.org using the heatmap2 function within gplots (see Table 5).

TABLE 5
Numerical data corresponding to the heatmap depicted in FIG 1A.

| | #conservation | | | | | | | | | | | | |
|---|---|---|---|---|---|---|---|---|---|---|---|---|---|
| | CFA/I | cs1 | cs2 | cs3 | cs4 | cs5 | cs6 | cs7 | cs8 | cs14 | cs17 | cs21 | nd |
| EtpA | 0.81 | 0.86 | 0.8 | 0.84 | 0.14 | 0 | 0.08 | 0.88 | 0 | 0.86 | 0.9 | 0.82 | 0.44 |
| EatA | 0.81 | 0.86 | 0.7 | 0.76 | 0.43 | 0.78 | 0.64 | 0.94 | 1 | 0.14 | 0.9 | 0.82 | 0.19 |
| either | 0.85 | 0.93 | 0.9 | 0.92 | 0.92 | 0.78 | 0.67 | 0.94 | 1 | 0.93 | 0.9 | 0.94 | 0.5 |

Recombinant Protein Production:

The antigens used in these studies were produced as polyhistidine-tagged recombinant proteins and purified by immobilized metal ion affinity chromatography (IMAC) as previously described [27,29,44,45]. Additional polishing steps including size exclusion or ion exchange chromatography were performed as needed to produce highly purified antigens. Purity of each antigen was assessed by SDS-PAGE followed by sensitive Coomassie Blue staining. Purified recombinant antigens were stored at −80° C.

Assessment of Immune Responses to Novel ETEC Virulence Proteins:

To quantify antibody concentrations directed at novel recombinant antigens, kinetic ELISA was performed on dilutions of plasma samples previously obtained from patients hospitalized at the International Centre for Diarrhoeal Disease Research in Dhaka, Bangladesh (icddr,b) with acute symptomatic ETEC infections. Plasma samples from non-infected adults and children obtained at icddr,b, or specimens obtained from children at Saint Louis Children's Hospital were used as negative controls. Samples from human volunteer ETEC H10407 challenge studies were kindly provided by Dr. Robert Gormely and Dr. Stephen Savarino of National Naval Medical Center, Bethesda Md.

Use of these clinical materials was approved by the Institutional Review Boards of both icddr,b and Washington University School of Medicine. All plasma samples were maintained at 4° C. in a humidified chamber prior to use in ELISA. Immune responses to purified recombinant proteins (rYghJ, rEaeH, rEtpA, rEatAₚ) were assessed by kinetic ELISA [46] as previously described [30,47]. Antigen binding to ELISA wells (Corning, Costar 2580) was first optimized to determine the optimal coating concentration and buffer system, using highly antigen-specific polyclonal rabbit antisera to detect binding by ELISA. Purified antigens were then diluted either in 50 mM carbonate buffer (pH 9.6) (rEtpA-myc-His₆, 1 μg/ml; rEatAₚ, 10 μg/ml; rYghJ-myc-His₆, 1 μg/ml); or in phosphate buffered saline (PBS, pH 7.4) (rEaeH-myc-His₆, 1 μg/ml). ELISA plate wells were coated with 100 μl/well overnight at 4° C., washed with PBS containing 0.05% Tween-20 (PBS-T), and blocked for 1 h at 37° C. with 1% BSA in PBS-T. All plasma samples were diluted at 1:4096 in blocking buffer. After incubation for 1 hour at 37° C., plates were washed with PBS-T, and secondary goat anti-human IgG(H+L)-HRP conjugated antibody (Pierce, 31410) was added at a final concentration of 1:10,000. After incubation for 30 minutes at 37° C., plates were washed and developed with TMB microwell peroxidase substrate [3,3′,5,5′-Tetramethylbenzidine] (KPL, 50-76-00). Kinetic absorbance measurements were determined at a wavelength of 650 nm, and acquired at 40 s intervals for 20 minutes using a microplate spectrophotometer (Eon, BioTek). All data were recorded and analyzed using Gen5 software (BioTek) and reported as the Vmax expressed as milliunits/min. Statistical calculations were performed using Prism v4.0c (GraphPad Software), using nonparametric Mann-Whitney (two-tailed) comparisons of data.

Mouse Immunization and Challenge Studies:

These studies were performed in strict accordance with the recommendations in the Guide for the Care and Use of Laboratory Animals of the National Institutes of Health, using an established protocol approved by the Washington University School of Medicine Animal Studies Committee.

Four groups of twelve CD-1 mice were immunized intranasally with either 1 μg of LT (adjuvant only controls), or 1 μg of LT+15 μg of rEatAₚ(H134R), or 1 μg of LT+15 μg of rEtpA, or 1 μg of LT+15 μg of rEatA(H134R)+15 μg of rEtpA on days 0, 14, 28. On day 40, mice were treated with streptomycin [5 g per liter] in drinking water for 24 hours, followed by drinking water alone for 18 hours. After administration of famotidine to reduce gastric acidity, mice were challenged with 10⁶ cfu of the kanamycin-resistant (lacZYA::KmR) strain jf876 [48] by oral gavage as previously described [47]. Fecal samples (6 pellets/mouse) were collected on day 42 before oral gavage, re-suspended in buffer (10 mM Tris, 100 mM NaCl, 0.05% Tween 20, 5 mM Sodium Azide, pH 7.4) overnight at 4° C., centrifuged to pellet insoluble material, and recover supernatant for fecal antibody testing (below). Twenty-four hours after infection, mice were sacrificed, sera were collected, and dilutions of saponin small-intestinal lysates were plated onto Luria agar plates containing kanamycin (50 μg/ml).

Murine immune responses to LT, EatA and EtpA were determined using previously described kinetic ELISA. Briefly, ELISA wells were coated with 1 μg/ml GM1, or 10 μg/ml of rEatAₚ(H134R), or 1 μg/ml rEtpA in carbonate buffer (15 mM Na₂CO₃, 35 mM NaHCO₃, 0.2 g/L NaN₃, pH8.6) overnight at 4° C. Wells were washed three times with phosphate-buffered saline containing 0.05% Tween 20 (PBS-T), blocked with 1% bovine serum albumin (BSA) in PBS-T for 1 h at 37° C., and 100 μl of fecal suspensions (undiluted) or sera (diluted 1:100 in PBS-T with 1% BSA) was added per ELISA well and incubated at 37° C. for 1 h. Horseradish peroxidase-conjugated secondary antibodies were used and signal detected with TMB (3,3′,5,5′-tetramethylbenzidine)-peroxidase substrate (KPL) substrate.

Ethics Statement:

All animal studies were performed under protocols approved by the Animal Studies Committee of Washington University School of Medicine (protocol number 20110246A1). All procedures complied with Public Health Service guidelines, and The Guide for the Care and Use of Laboratory Animals.

All human studies included were performed under a protocol approved by the Institutional Review Board of Washington University School of Medicine (IRB ID#201110126). All of the human studies here report anonymous analysis of de-identified pre-existing sera previously stored from earlier studies for which no additional consent was obtained.

TABLE 6
EtpA, EatA, CF, LT, and ST prevalence.

| Stock | | Strain | | isolation | | | | | | |
|---|---|---|---|---|---|---|---|---|---|---|
| # (jf) | severity | designation | origin | year | EatA | EtpA | CFs | LT | ST | cfa/1 |
| 350 | 2 | ETEC | USA, | 1975 | 1 | 1 | | 1 | 1 | |
| | | Throop | Texas | | | | | | | |
| | | D “1” | | | | | | | | |
| 1388 | 2 | ETEC | Amazon, | 1998 | 1 | 0 | 1 | 1 | 1 | |
| | | Envira | Brazil | | | | | | | |
| | | 8/11 | | | | | | | | |
| 1389 | 2 | ETEC | Amazon, | 1998 | 1 | 0 | 1 | 1 | 1 | |
| | | Envira | Brazil | | | | | | | |
| | | 10/1 | | | | | | | | |
| 1390 | 2 | ETEC | Amazon, | 1998 | 1 | 1 | 1 | 1 | 0 | |
| | | Jurua | Brazil | | | | | | | |
| | | 18/11 | | | | | | | | |
| 1391 | 2 | ETEC | Amazon, | 1998 | 1 | 1 | 1 | 1 | 0 | |
| | | Jurua | Brazil | | | | | | | |
| | | 20/10 | | | | | | | | |
| 2007 | 2 | P02997067.1 | Bangladesh | 2011 | 1 | 0 | | 1 | 1 | |
| 2021 | 2 | MP021552.1 | Bangladesh | 2011 | 1 | 0 | | 1 | 1 | |
| 2036 | 2 | MP021561.1 | Bangladesh | 2011 | 1 | 0 | | 0 | 1 | |
| 2053 | 2 | MP021017.1 | Bangladesh | 2011 | 0 | 0 | | 0 | 1 | |
| 2063 | 2 | MP020980.1 | Bangladesh | 2011 | 1 | 0 | | 0 | 1 | |
| 2097 | 1 | 2720900 | Bangladesh | 2007 | 1 | 1 | 1 | 0 | 1 | 1 |
| 2098 | 1 | 2719100 | Bangladesh | 2007 | 1 | 1 | 1 | 0 | 1 | 1 |
| 2099 | 1 | 2722950 | Bangladesh | 2007 | 1 | 1 | 1 | 1 | 1 | 1 |
| 2100 | 1 | 2726800 | Bangladesh | 2007 | 0 | 1 | 1 | 1 | 1 | |
| 2101 | 1 | 2726950 | Bangladesh | 2007 | 1 | 1 | 1 | 0 | 1 | |
| 2102 | 1 | 2729250 | Bangladesh | 2007 | 1 | 0 | 1 | 1 | 1 | 1 |
| 2103 | 2 | 2747800 | Bangladesh | 2007 | 1 | 0 | 1 | 1 | 1 | |
| 2104 | 2 | 2741950 | Bangladesh | 2007 | 1 | 1 | 1 | 1 | 1 | |
| 2105 | 2 | 2735000 | Bangladesh | 2007 | 1 | 1 | 1 | 1 | 0 | |
| 2106 | 2 | 2730350 | Bangladesh | 2007 | 0 | 1 | 1 | 1 | 1 | 1 |
| 2107 | 2 | 2730450 | Bangladesh | 2007 | 1 | 1 | 1 | 1 | 1 | |
| 2108 | 2 | 2770900 | Bangladesh | 2007 | 1 | 0 | 1 | 1 | 1 | |
| 2109 | 2 | 2780750 | Bangladesh | 2007 | 0 | 0 | 0 | 0 | 1 | |
| 2110 | 2 | 2731150 | Bangladesh | 2007 | 1 | 0 | 1 | 1 | 0 | |
| 2111 | 2 | 2785200 | Bangladesh | 2007 | 0 | 0 | 0 | 1 | 0 | |
| 2112 | 2 | 2733950 | Bangladesh | 2007 | 1 | 0 | 1 | 1 | 1 | |
| 2113 | 2 | 2749250 | Bangladesh | 2007 | 1 | 0 | 1 | 1 | 1 | |
| 2114 | 2 | 2756500 | Bangladesh | 2007 | 1 | 1 | 1 | 1 | 0 | |
| 2115 | 2 | 2788150 | Bangladesh | 2007 | 0 | 0 | 0 | 1 | 1 | |
| 2116 | 2 | 2762100 | Bangladesh | 2007 | 1 | 0 | 1 | 1 | 0 | |
| 2117 | 1 | 2845350 | Bangladesh | 2008 | 0 | 0 | 1 | 1 | 1 | |
| 2118 | 1 | 2845650 | Bangladesh | 2008 | 0 | 0 | 1 | 1 | 1 | |
| 2119 | 1 | 2846750 | Bangladesh | 2008 | 1 | 1 | 1 | 1 | 1 | |
| 2120 | 1 | 2848050 | Bangladesh | 2008 | 0 | 0 | 1 | 1 | 1 | |
| 2121 | 2 | 2850400 | Bangladesh | 2008 | 1 | 1 | 1 | 1 | 0 | |
| 2122 | 2 | 2850750 | Bangladesh | 2008 | 1 | 1 | 1 | 1 | 0 | |
| 2123 | 1 | 2851500 | Bangladesh | 2008 | 1 | 1 | 1 | 1 | 0 | |
| 2124 | 2 | 2853500 | Bangladesh | 2008 | 1 | 1 | 0 | 1 | 0 | |
| 2125 | 2 | 2854350 | Bangladesh | 2008 | 0 | 1 | 1 | 1 | 0 | |
| 2126 | 2 | 2860050 | Bangladesh | 2008 | 1 | 0 | 1 | 1 | 1 | |
| 2127 | 2 | 2860650 | Bangladesh | 2008 | 1 | 0 | 1 | 1 | 1 | |
| 2128 | 2 | 2861200 | Bangladesh | 2008 | 0 | 0 | 1 | 0 | 1 | |
| 2129 | 2 | 2862600 | Bangladesh | 2008 | 0 | 0 | 1 | 0 | 1 | |
| 2130 | 2 | 2863500 | Bangladesh | 2008 | 0 | 0 | 1 | 0 | 1 | 1 |
| 2131 | 2 | 2864350 | Bangladesh | 2008 | 0 | 0 | 0 | 1 | 1 | |
| 2132 | 2 | 2865200 | Bangladesh | 2008 | 0 | 0 | 0 | 0 | 1 | |
| 2133 | 2 | 2866350 | Bangladesh | 2008 | 1 | 0 | 1 | 1 | 1 | |
| 2134 | 2 | 2866450 | Bangladesh | 2008 | 1 | 1 | 1 | 1 | 0 | |
| 2135 | 2 | 2866550 | Bangladesh | 2008 | 1 | 1 | 1 | 1 | 0 | |
| 2136 | 2 | 2866750 | Bangladesh | 2008 | 1 | 1 | 1 | 1 | 0 | |
| 2137 | 2 | 2867750 | Bangladesh | 2008 | 1 | 1 | 1 | 1 | 0 | |
| 2138 | 2 | 2871950 | Bangladesh | 2008 | 1 | 1 | 1 | 1 | 0 | |
| 2139 | 2 | 2872000 | Bangladesh | 2008 | 1 | 1 | 1 | 1 | 0 | |
| 2140 | 2 | 2872800 | Bangladesh | 2008 | 1 | 1 | 1 | 1 | 0 | |
| 2141 | 2 | 2875000 | Bangladesh | 2008 | 1 | 1 | 1 | 1 | 0 | |
| 2142 | 2 | 2875150 | Bangladesh | 2008 | 0 | 0 | 1 | 1 | 1 | |
| 2143 | 2 | 199900.1 | Bangladesh | 2011 | 1 | 1 | 1 | 1 | 0 | |
| 2144 | 2 | 199900.2 | Bangladesh | 2011 | 1 | 1 | 1 | 1 | 0 | |
| 2145 | 2 | 21600.1 | Bangladesh | 2011 | 0 | 1 | 1 | 1 | 1 | |
| 2146 | 2 | 21600.2 | Bangladesh | 2011 | 0 | 1 | 1 | 1 | 1 | |
| 2147 | 2 | 21600.3 | Bangladesh | 2011 | 0 | 1 | 1 | 1 | 1 | |
| 2148 | 2 | 21600.4 | Bangladesh | 2011 | 0 | 1 | 1 | 1 | 1 | |
| 2149 | 0 | BCE006, | Bangladesh | 2004 | 1 | 1 | 1 | 1 | 1 | |
| | | MS023 | | | | | | | | |
| 2150 | 0 | BCE011, | Bangladesh | 2002 | 0 | 0 | 1 | 0 | 1 | 1 |
| | | MS001 | | | | | | | | |
| 2151 | 0 | BCE019, | Bangladesh | 2003 | 1 | 1 | 1 | 1 | 1 | |
| | | MS013 | | | | | | | | |
| 2152 | 0 | BCE030, | Bangladesh | 2003 | 1 | 1 | 1 | 0 | 1 | 1 |
| | | MS009 | | | | | | | | |
| 2153 | 0 | BCE032, | Bangladesh | 2003 | 1 | 1 | 1 | 1 | 1 | |
| | | MS012 | | | | | | | | |
| 2154 | 0 | BCE034, | Bangladesh | 2003 | 1 | 1 | 1 | 0 | 1 | 1 |
| | | MS014 | | | | | | | | |
| 2155 | 2 | P0304816.1 | Bangladesh | 2011 | 1 | 0 | | 0 | 1 | |
| 2170 | 2 | P0305260.1 | Bangladesh | 2011 | 0 | 1 | | 0 | 1 | |
| 2184 | 2 | p0305293.1 | Bangladesh | 2011 | 1 | 0 | | 1 | 1 | |
| 2199 | 2 | p0301867.1 | Bangladesh | 2011 | 1 | 1 | | 0 | 1 | |
| 2208 | 2 | p0302293.2 | Bangladesh | 2011 | 0 | 1 | | 1 | 1 | |
| 2216 | 2 | p0302308.1 | Bangladesh | 2011 | 0 | 1 | | 1 | 1 | |
| 2226 | 2 | p0304777.1 | Bangladesh | 2011 | 1 | 1 | | 1 | 0 | |
| 2267 | 2 | MP020940.1 | Bangladesh | 2011 | 0 | 0 | | 0 | 1 | |
| 2282 | 2 | P0298942.1 | Bangladesh | 2011 | 1 | 0 | | 1 | 1 | |
| 2295 | 2 | p0299438.2 | Bangladesh | 2011 | 0 | 1 | | 0 | 1 | |
| 2305 | 2 | p0299917.1 | Bangladesh | 2011 | 0 | 0 | | 1 | 1 | |
| 2315 | 2 | ETP050002 | Bangladesh | 2005 | 1 | 1 | 1 | 0 | 1 | 1 |
| 2316 | 2 | ETP050003 | Bangladesh | 2005 | 0 | 0 | 0 | 0 | 1 | |
| 2317 | 2 | ETP050007 | Bangladesh | 2005 | 0 | 0 | 1 | 1 | 1 | |
| 2318 | 2 | ETP050008 | Bangladesh | 2005 | 0 | 1 | 0 | 0 | 1 | |
| 2319 | 2 | ETP050009 | Bangladesh | 2005 | 0 | 0 | 1 | 1 | 1 | |
| 2320 | 2 | ETP050010 | Bangladesh | 2005 | 0 | 1 | 1 | 1 | 1 | |
| 2321 | 2 | ETP050011 | Bangladesh | 2005 | 0 | 1 | 1 | 1 | 1 | |
| 2322 | 2 | ETP050012 | Bangladesh | 2005 | 0 | 0 | 1 | 0 | 1 | |
| 2323 | 2 | ETP050015 | Bangladesh | 2005 | 0 | 0 | 1 | 1 | 1 | |
| 2324 | 2 | ETP050016 | Bangladesh | 2005 | 0 | 1 | 0 | 0 | 1 | |
| 2325 | 2 | ETP050017 | Bangladesh | 2005 | 1 | 0 | 1 | 1 | 1 | |
| 2326 | 2 | ETP050019 | Bangladesh | 2005 | 0 | 1 | 0 | 1 | 1 | |
| 2327 | 2 | ETP05020 | Bangladesh | 2005 | 0 | 0 | 1 | 1 | 0 | |
| 2328 | 2 | ETP05026 | Bangladesh | 2005 | 1 | 1 | 1 | 1 | 1 | 1 |
| 2329 | 2 | ETP050035 | Bangladesh | 2005 | 0 | 0 | 0 | 1 | 1 | |
| 2330 | 2 | ETP050038 | Bangladesh | 2005 | 0 | 0 | 1 | 1 | 1 | |
| 2331 | 2 | ETP050039 | Bangladesh | 2005 | 0 | 1 | 1 | 1 | 1 | |
| 2332 | 2 | ETP050044 | Bangladesh | 2005 | 0 | 0 | 1 | 0 | 1 | 1 |
| 2333 | 2 | ETP050046 | Bangladesh | 2005 | 1 | 1 | 1 | 0 | 1 | 1 |
| 2334 | 2 | ETP050047 | Bangladesh | 2005 | 0 | 0 | 0 | 1 | 1 | |
| 2335 | 2 | ETP050050 | Bangladesh | 2005 | 0 | 0 | 1 | 1 | 0 | |
| 2336 | 2 | 174750 | Bangladesh | 2010 | 1 | 0 | 1 | 1 | 1 | |
| 2337 | 2 | 174900 | Bangladesh | 2010 | 0 | 0 | 0 | 0 | 1 | |
| 2338 | 2 | 178200 | Bangladesh | 2010 | 0 | 0 | 0 | 0 | 1 | |
| 2339 | 2 | 178850 | Bangladesh | 2010 | 1 | 0 | 0 | 1 | 1 | |
| 2340 | 2 | 178900 | Bangladesh | 2010 | 1 | 1 | 1 | 0 | 1 | 1 |
| 2341 | 2 | 179100 | Bangladesh | 2010 | 0 | 0 | 0 | 1 | 1 | |
| 2342 | 2 | 179550 | Bangladesh | 2010 | 1 | 1 | 1 | 1 | 0 | |
| 2343 | 2 | 180200 | Bangladesh | 2010 | 1 | 1 | 1 | 1 | 1 | |
| 2344 | 2 | 180600 | Bangladesh | 2010 | 1 | 1 | 1 | 1 | 1 | |
| 2345 | 2 | 180050 | Bangladesh | 2010 | 1 | 1 | 1 | 1 | 0 | |
| 2346 | 2 | C034666 | Bangladesh | 2010 | 1 | 0 | 0 | 1 | 1 | |
| 2347 | 2 | C035134 | Bangladesh | 2010 | 1 | 1 | 1 | 1 | 1 | |
| 2348 | 2 | C035209 | Bangladesh | 2010 | 0 | 0 | 1 | 0 | 1 | |
| 2349 | 2 | C035213 | Bangladesh | 2010 | 1 | 1 | 1 | 1 | 1 | |
| 2350 | 2 | C035605 | Bangladesh | 2010 | 1 | 1 | 1 | 1 | 0 | |
| 2351 | 2 | C035662 | Bangladesh | 2010 | 0 | 0 | 0 | 1 | 1 | |
| 2352 | 2 | C035776 | Bangladesh | 2010 | 1 | 0 | 1 | 0 | 1 | |
| 2353 | 2 | C035959 | Bangladesh | 2010 | 0 | 1 | 1 | 1 | 1 | |
| 2354 | 2 | C036255 | Bangladesh | 2010 | 0 | 1 | 1 | 1 | 1 | |
| 2355 | 2 | 173150 | Bangladesh | 2010 | 0 | 0 | 1 | 1 | 1 | |
| 2356 | 0 | BCE001, | Bangladesh | 2002 | 0 | 1 | 0 | 1 | 0 | |
| | | MS16 | | | | | | | | |
| 2357 | 0 | BCE002, | Bangladesh | 2002 | 0 | 1 | 1 | 1 | 1 | |
| | | MS12 | | | | | | | | |
| 2358 | 0 | BCE005, | Bangladesh | 2002 | 0 | 1 | 0 | 1 | 1 | |
| | | MS23 | | | | | | | | |
| 2359 | 0 | BCE007, | Bangladesh | 2002 | 1 | 1 | 1 | 1 | 1 | |
| | | MS11 | | | | | | | | |
| 2360 | 0 | BCE008, | Bangladesh | 2002 | 0 | 0 | 0 | 1 | 0 | |
| | | MS1 | | | | | | | | |
| 2361 | 0 | BCE008, | Bangladesh | 2002 | 0 | 0 | 1 | 0 | 1 | |
| | | MS13 | | | | | | | | |
| 2362 | 0 | BCE019, | Bangladesh | 2002 | 1 | 1 | 1 | 1 | 1 | |
| | | MS16 | | | | | | | | |
| 2363 | 0 | BCE035, | Bangladesh | 2002 | 1 | 0 | 1 | 1 | 0 | |
| | | MS8 | | | | | | | | |
| 2364 | 0 | BCE039, | Bangladesh | 2002 | 0 | 0 | 0 | 1 | 0 | |
| | | MS13 | | | | | | | | |
| 2365 | 0 | BCE041, | Bangladesh | 2002 | 0 | 0 | 0 | 1 | 0 | |
| | | MS11 | | | | | | | | |
| 2366 | 0 | BCE046, | Bangladesh | 2002 | 0 | 0 | 0 | 1 | 0 | |
| | | MS16 | | | | | | | | |
| 2367 | 0 | BCE049, | Bangladesh | 2002 | 1 | 0 | 1 | 1 | 1 | |
| | | MS9 | | | | | | | | |
| 2368 | 0 | BCE054, | Bangladesh | 2002 | 0 | 0 | 1 | 1 | 1 | |
| | | MS24 | | | | | | | | |
| 2369 | 0 | BCE058, | Bangladesh | 2002 | 1 | 1 | 1 | 0 | 1 | 1 |
| | | MS13 | | | | | | | | |
| 2370 | 0 | BCE062, | Bangladesh | 2002 | 1 | 1 | 1 | 1 | 1 | |
| | | MS24 | | | | | | | | |
| 2371 | 0 | BCE063, | Bangladesh | 2002 | 0 | 1 | 0 | 1 | 0 | |
| | | MS14 | | | | | | | | |
| 2372 | 0 | BCE068, | Bangladesh | 2002 | 0 | 1 | 1 | 1 | 1 | |
| | | MS10 | | | | | | | | |
| 2373 | 0 | BCE068, | Bangladesh | 2002 | 0 | 1 | 0 | 0 | 1 | |
| | | MS23 | | | | | | | | |
| 2374 | 0 | BCE069, | Bangladesh | 2002 | 1 | 1 | 1 | 1 | 1 | 1 |
| | | MS15 | | | | | | | | |
| 2375 | 0 | BCE069, | Bangladesh | 2002 | 1 | 1 | 1 | 1 | 1 | |
| | | MS9 | | | | | | | | |
| 2381 | 2 | mp021566.1 | Bangladesh | 2011 | 1 | 1 | | 1 | 1 | |
| 2409 | 2 | ETP98109 | Bangladesh | 1998 | 1 | 1 | 1 | 0 | 1 | 1 |
| 2410 | 2 | ETP98111 | Bangladesh | 1998 | 1 | 1 | 1 | 1 | 1 | |
| 2411 | 2 | ETP98112 | Bangladesh | 1998 | 1 | 1 | 1 | 1 | 1 | |
| 2412 | 2 | ETP98114 | Bangladesh | 1998 | 0 | 1 | 1 | 0 | 1 | |
| 2413 | 2 | ETP98115 | Bangladesh | 1998 | 0 | 1 | 1 | 1 | 1 | |
| 2414 | 2 | ETP98068 | Bangladesh | 1998 | 1 | 1 | 1 | 1 | 1 | 1 |
| 2415 | 2 | ETP98073 | Bangladesh | 1998 | 1 | 0 | 1 | 1 | 1 | |
| 2416 | 2 | ETP98097 | Bangladesh | 1998 | 1 | 1 | 1 | 1 | 1 | |
| 2417 | 2 | ETP98103 | Bangladesh | 1998 | 1 | 1 | 1 | 1 | 1 | |
| 2418 | 2 | ETP98105 | Bangladesh | 1998 | 1 | 1 | 1 | 1 | 1 | |
| 2419 | 2 | ETP98053 | Bangladesh | 1998 | 1 | 0 | 1 | 1 | 1 | |
| 2420 | 2 | ETP98056 | Bangladesh | 1998 | 1 | 0 | 1 | 0 | 1 | |
| 2421 | 2 | ETP98061 | Bangladesh | 1998 | 1 | 1 | 1 | 0 | 1 | |
| 2422 | 2 | ETP98062 | Bangladesh | 1998 | 1 | 0 | 1 | 0 | 1 | |
| 2423 | 2 | ETP98066 | Bangladesh | 1998 | 0 | 0 | 1 | 1 | 1 | |
| 2424 | 2 | ETP98004 | Bangladesh | 1998 | 0 | 0 | 0 | 0 | 1 | |
| 2425 | 2 | ETP98015 | Bangladesh | 1998 | 1 | 0 | 1 | 0 | 1 | |
| 2426 | 2 | ETP98028 | Bangladesh | 1998 | 1 | 0 | 1 | 0 | 1 | |
| 2427 | 2 | ETP98038 | Bangladesh | 1998 | 1 | 1 | 1 | 0 | 1 | 1 |
| 2428 | 2 | ETP98042 | Bangladesh | 1998 | 1 | 0 | 1 | 1 | 1 | |
| 2430 | 1 | BCE003, | Bangladesh | 2002 | 1 | 1 | 0 | 1 | 1 | |
| | | DS5 | | | | | | | | |
| 2431 | 1 | BCE021, | Bangladesh | 2002 | 0 | 1 | 0 | 0 | 1 | |
| | | DS7 | | | | | | | | |
| 2432 | 1 | BCE046, | Bangladesh | 2002 | 1 | 0 | 1 | 1 | 1 | |
| | | DS7 | | | | | | | | |
| 2433 | 1 | BCE062, | Bangladesh | 2002 | 1 | 1 | 0 | 1 | 1 | |
| | | DS2 | | | | | | | | |
| 2434 | 1 | BCE007, | Bangladesh | 2002 | 1 | 0 | 1 | 1 | 1 | |
| | | DS5 | | | | | | | | |
| 2435 | 1 | BCE022, | Bangladesh | 2002 | 1 | 1 | 1 | 0 | 1 | 1 |
| | | DS6 | | | | | | | | |
| 2436 | 1 | BCE049, | Bangladesh | 2002 | 1 | 1 | 0 | 1 | 1 | |
| | | DS3 | | | | | | | | |
| 2437 | 1 | BCE063, | Bangladesh | 2002 | 0 | 1 | 1 | 1 | 1 | |
| | | DS4 | | | | | | | | |
| 2438 | 1 | BCE011, | Bangladesh | 2002 | 0 | 1 | 0 | 1 | 0 | |
| | | DS3 | | | | | | | | |
| 2439 | 1 | BCE035, | Bangladesh | 2002 | 1 | 1 | 1 | 1 | 1 | |
| | | DS6 | | | | | | | | |
| 2440 | 1 | BCE054, | Bangladesh | 2002 | 1 | 0 | 1 | 1 | 1 | |
| | | DS4 | | | | | | | | |
| 2441 | 1 | BCE066, | Bangladesh | 2002 | 0 | 1 | 0 | 1 | 0 | |
| | | DS5 | | | | | | | | |
| 2442 | 1 | BCE013, | Bangladesh | 2002 | 1 | 1 | 1 | 0 | 1 | 1 |
| | | DS1 | | | | | | | | |
| 2443 | 1 | BCE039, | Bangladesh | 2002 | 1 | 0 | 1 | 1 | 1 | |
| | | DS2 | | | | | | | | |
| 2444 | 1 | BCE055, | Bangladesh | 2002 | 1 | 1 | 1 | 1 | 0 | |
| | | DS1 | | | | | | | | |
| 2445 | 1 | BCE069, | Bangladesh | 2002 | 1 | 1 | 1 | 1 | 1 | |
| | | DS2 | | | | | | | | |
| 2446 | 1 | BCE018, | Bangladesh | 2002 | 1 | 1 | 0 | 1 | 1 | |
| | | DS6 | | | | | | | | |
| 2447 | 1 | BCE046, | Bangladesh | 2002 | 0 | 1 | 0 | 1 | 0 | |
| | | DS2 | | | | | | | | |
| 2448 | 1 | BCE061, | Bangladesh | 2002 | 0 | 0 | 0 | 1 | 1 | |
| | | DS1 | | | | | | | | |
| 2449 | 1 | BCE129, | Bangladesh | 2002 | 0 | 1 | | 0 | 1 | |
| | | DS2 | | | | | | | | |
| totals | | | | | 106 | 102 | 126 | 134 | 142 | 21 |

| Stock | | | | | | | | | | | | | |
|---|---|---|---|---|---|---|---|---|---|---|---|---|---|
| # (jf) | cs1 | cs2 | cs3 | cs4 | cs5 | cs6 | cs7 | cs8 | cs14 | cs17 | cs21 | CF0ve | EtpA/EatA |
| 350 | | | | | | | | | | | | | 2 |
| 1388 | | | | | 1 | 1 | | | | | | | 1 |
| 1389 | | | | | 1 | 1 | | | | | | | 1 |
| 1390 | | | | | | | 1 | | | | | | 2 |
| 1391 | | | | | | | 1 | | | | | | 2 |
| 2007 | | | | | | | | | | | | | 1 |
| 2021 | | | | | | | | | | | | | 1 |
| 2036 | | | | | | | | | | | | | 1 |
| 2053 | | | | | | | | | | | | | 0 |
| 2063 | | | | | | | | | | | | | 1 |
| 2097 | | | | | | | | | | | | | 2 |
| 2098 | | | | | | | | | | | 1 | | 2 |
| 2099 | | | | | | | | | | | | | 2 |
| 2100 | | | | | | | | | 1 | | | | 1 |
| 2101 | | | | | | | | | | | 1 | | 2 |
| 2102 | | | | | | | | | | | | | 1 |
| 2103 | | | | | 1 | 1 | | | | | | | 1 |
| 2104 | 1 | | 1 | | | | | | | | 1 | | 2 |
| 2105 | | | | | | | 1 | | | | | | 2 |
| 2106 | | | | | | | | | | | 1 | | 1 |
| 2107 | 1 | | 1 | | | | | | | | 1 | | 2 |
| 2108 | | | | | 1 | 1 | | | | | | | 1 |
| 2109 | | | | | | | | | | | | 1 | 0 |
| 2110 | | | | | | 1 | | 1 | | | | | 1 |
| 2111 | | | | | | | | | | | | 1 | 0 |
| 2112 | | | | 1 | | 1 | | | | | | | 1 |
| 2113 | | | | | 1 | 1 | | | | | | | 1 |
| 2114 | | | | | | | 1 | | | | | | 2 |
| 2115 | | | | | | | | | | | | 1 | 0 |
| 2116 | | | | | | 1 | | 1 | | | | | 1 |
| 2117 | | | | 1 | | 1 | | | | | | | 0 |
| 2118 | | | | 1 | | 1 | | | | | | | 0 |
| 2119 | | 1 | 1 | | | | | | | | | | 2 |
| 2120 | | | | | 1 | 1 | | | | | | | 0 |
| 2121 | | | | | | | 1 | | | | | | 2 |
| 2122 | | | | | | | | | | 1 | | | 2 |
| 2123 | | | | | | | 1 | | | | | | 2 |
| 2124 | | | | | | | | | | | | 1 | 2 |
| 2125 | | | | | | | | | 1 | | | | 1 |
| 2126 | | | | | 1 | 1 | | | | | | | 1 |
| 2127 | | | | | 1 | 1 | | | | | | | 1 |
| 2128 | | | | | | 1 | | | | | | | 0 |
| 2129 | | | | 1 | | 1 | | | | | | | 0 |
| 2130 | | | | | | | | | | | | | 0 |
| 2131 | | | | | | | | | | | | 1 | 0 |
| 2132 | | | | | | | | | | | | 1 | 0 |
| 2133 | | | | | 1 | 1 | | | | | | | 1 |
| 2134 | | | | | | | 1 | | | | | | 2 |
| 2135 | | | | | | | 1 | | | | | | 2 |
| 2136 | | | | | | | 1 | | | | | | 2 |
| 2137 | | | | | | | 1 | | | | | | 2 |
| 2138 | | 1 | 1 | | | | | | | | | | 2 |
| 2139 | | 1 | 1 | | | | | | | | | | 2 |
| 2140 | | | | | | | 1 | | | | | | 2 |
| 2141 | | | | | | | 1 | | | | | | 2 |
| 2142 | | | | 1 | | 1 | | | | | | | 0 |
| 2143 | | | | | | | | | | 1 | | | 2 |
| 2144 | | | | | | | | | | 1 | | | 2 |
| 2145 | | | | | | | | | 1 | | | | 1 |
| 2146 | | | | | | | | | 1 | | | | 1 |
| 2147 | | | | | | | | | 1 | | | | 1 |
| 2148 | | | | | | | | | 1 | | | | 1 |
| 2149 | 1 | | 1 | | | | | | | | | | 2 |
| 2150 | | | | | | | | | | | | | 0 |
| 2151 | 1 | | 1 | | | | | | | | | | 2 |
| 2152 | | | | | | | | | | | | | 2 |
| 2153 | 1 | | 1 | | | | | | | | | | 2 |
| 2154 | | | | | | | | | | | | | 2 |
| 2155 | | | | | | | | | | | | | 1 |
| 2170 | | | | | | | | | | | | | 1 |
| 2184 | | | | | | | | | | | | | 1 |
| 2199 | | | | | | | | | | | | | 2 |
| 2208 | | | | | | | | | | | | | 1 |
| 2216 | | | | | | | | | | | | | 1 |
| 2226 | | | | | | | | | | | | | 2 |
| 2267 | | | | | | | | | | | | | 0 |
| 2282 | | | | | | | | | | | | | 1 |
| 2295 | | | | | | | | | | | | | 1 |
| 2305 | | | | | | | | | | | | | 0 |
| 2315 | | | | | | | | | | | | | 2 |
| 2316 | | | | | | | | | | | | 1 | 0 |
| 2317 | | | | | 1 | 1 | | | | | | | 0 |
| 2318 | | | | | | | | | | | | 1 | 1 |
| 2319 | | | | | 1 | 1 | | | | | | | 0 |
| 2320 | | | 1 | | | | | | | | 1 | | 1 |
| 2321 | | 1 | 1 | | | | | | | | | | 1 |
| 2322 | | | | | | 1 | | | | | | | 0 |
| 2323 | | | | | 1 | 1 | | | | | | | 0 |
| 2324 | | | | | | | | | | | | 1 | 1 |
| 2325 | | | | | 1 | 1 | | | | | | | 1 |
| 2326 | | | | | | | | | | | | 1 | 1 |
| 2327 | | | | | | | 1 | | | | | | 0 |
| 2328 | | | | | | | | | | | | | 2 |
| 2329 | | | | | | | | | | | | 1 | 0 |
| 2330 | | | | | 1 | 1 | | | | | | | 0 |
| 2331 | | | | | | | | | 1 | | | | 1 |
| 2332 | | | | | | | | | | | | | 0 |
| 2333 | | | | | | | | | | | 1 | | 2 |
| 2334 | | | | | | | | | | | | 1 | 0 |
| 2335 | | | | | | | | | | 1 | | | 0 |
| 2336 | 1 | | 1 | | | | | | | | 1 | | 1 |
| 2337 | | | | | | | | | | | | 1 | 0 |
| 2338 | | | | | | | | | | | | 1 | 0 |
| 2339 | | | | | | | | | | | | 1 | 1 |
| 2340 | | | | | | | | | | | 1 | | 2 |
| 2341 | | | | | | | | | | | | 1 | 0 |
| 2342 | | | | | | | 1 | | | | | | 2 |
| 2343 | | | | | | | 1 | | | | | | 2 |
| 2344 | | | | | | | | | | 1 | | | 2 |
| 2345 | | | | | | | | | | 1 | | | 2 |
| 2346 | | | | | | | | | | | | 1 | 1 |
| 2347 | | | | | | | | | | 1 | | | 2 |
| 2348 | | 1 | 1 | | | | | | | | | | 0 |
| 2349 | | | | | | | | | | 1 | | | 2 |
| 2350 | | | | | | | | | | 1 | | | 2 |
| 2351 | | | | | | | | | | | | 1 | 0 |
| 2352 | | | | 1 | | 1 | | | | | | | 1 |
| 2353 | | | | | | | | | 1 | | | | 1 |
| 2354 | | | | | | | | | 1 | | | | 1 |
| 2355 | 1 | | 1 | | | | | | | | 1 | | 0 |
| 2356 | | | | | | | | | | | | 1 | 1 |
| 2357 | 1 | | 1 | | | | | | | | | | 1 |
| 2358 | | | | | | | | | | | | 1 | 1 |
| 2359 | 1 | | 1 | | | | | | | | 1 | | 2 |
| 2360 | | | | | | | | | | | | 1 | 0 |
| 2361 | | | | | | 1 | | | | | | | 0 |
| 2362 | | 1 | 1 | | | | | | | | | | 2 |
| 2363 | | | | | | | 1 | | | | | | 1 |
| 2364 | | | | | | | | | | | | 1 | 0 |
| 2365 | | | | | | | | | | | | 1 | 0 |
| 2366 | | | | | | | | | | | | 1 | 0 |
| 2367 | | 1 | 1 | | | | | | | | 1 | | 1 |
| 2368 | | | | | | | | | 1 | | | | 0 |
| 2369 | | | | | | | | | | | | | 2 |
| 2370 | | 1 | 1 | | | | | | | | | | 2 |
| 2371 | | | | | | | | | | | | 1 | 1 |
| 2372 | | 1 | 1 | | | | | | | | | | 1 |
| 2373 | | | | | | | | | | | | 1 | 1 |
| 2374 | | | | | | | | | | | | | 2 |
| 2375 | 1 | | 1 | | | | | | | | 1 | | 2 |
| 2381 | | | | | | | | | | | | | 2 |
| 2409 | | | | | | | | | | | | | 2 |
| 2410 | | | | 1 | | 1 | | | | | | | 2 |
| 2411 | 1 | | 1 | | | | | | | | 1 | | 2 |
| 2412 | | | | | | | | | 1 | | | | 1 |
| 2413 | | | | | | 1 | | | | | | | 1 |
| 2414 | | | | | | | | | | | | | 2 |
| 2415 | | | | | 1 | 1 | | | | | | | 1 |
| 2416 | | | | | | | | | 1 | | | | 2 |
| 2417 | 1 | | 1 | | | | | | | | 1 | | 2 |
| 2418 | 1 | | 1 | | | | | | | | 1 | | 2 |
| 2419 | | | | | 1 | 1 | | | | | | | 1 |
| 2420 | | | | | | 1 | | | | | | | 1 |
| 2421 | | | | | | 1 | | | | | | | 2 |
| 2422 | | | | | | | | | 1 | | | | 1 |
| 2423 | | | | | | 1 | | | | | | | 0 |
| 2424 | | | | | | | | | | | | 1 | 0 |
| 2425 | | | | | 1 | 1 | | | | | | | 1 |
| 2426 | | | | | 1 | 1 | | | | | | | 1 |
| 2427 | | | | | | | | | | | | | 2 |
| 2428 | | | | | 1 | 1 | | | | | | | 1 |
| 2430 | | | | | | | | | | | | 1 | 2 |
| 2431 | | | | | | | | | | | | 1 | 1 |
| 2432 | | | | | 1 | 1 | | | | | | | 1 |
| 2433 | | | | | | | | | | | | 1 | 2 |
| 2434 | | | | | 1 | 1 | | | | | | | 1 |
| 2435 | | | | | | | | | | | | | 2 |
| 2436 | | | | | | | | | | | | 1 | 2 |
| 2437 | | | | | | | | | 1 | | | | 1 |
| 2438 | | | | | | | | | | | | 1 | 1 |
| 2439 | | 1 | 1 | | | | | | | | | | 2 |
| 2440 | | | | | 1 | 1 | | | | | | | 1 |
| 2441 | | | | | | | | | | | | 1 | 1 |
| 2442 | | | | | | | | | | | | | 2 |
| 2443 | | | | | 1 | 1 | | | | | | | 1 |
| 2444 | | | | | | | | | | 1 | | | 2 |
| 2445 | 1 | | 1 | | | | | | | | 1 | | 2 |
| 2446 | | | | | | | | | | | | 1 | 2 |
| 2447 | | | | | | | | | | | | 1 | 1 |
| 2448 | | | | | | | | | | | | 1 | 0 |
| 2449 | | | | | | | | | | | | | 1 |
| totals | 14 | 10 | 25 | 7 | 23 | 39 | 16 | 2 | 14 | 10 | 17 | 36 | |

column A: frozen stock number in the Fleckenstein laboratory (used as internal reference and for ordering strains from the lab)
column B: disease severity 0 = asymptomatic; 1 = mild disease; 2 = severe, cholera-like illness
column C: strain designation as originally defined
column D: country of origin
column E: date of isolation (year)
column F: EatA passenger domain detection in culture supernatant by immunoblotting 0 = no; 1 = yes
column G: EtpA detection in culture supernatant by immunoblotting 0 = no; 1 = yes
column H: CF detected 0 = no; 1 = yes
column I: LT detected (PCR) 0 = no; 1 = yes
column J: ST detected (PCR) 0 = no; 1 = yes
columns K-V: presence of given individual CFs; 0 = no; 1 = yes

TABLE 7
Distribution of EtpA and EatA in CF+ strains.

| | CF | | | | | | | | | | | | |
|---|---|---|---|---|---|---|---|---|---|---|---|---|---|
| | cfa/i | cs1 | cs2 | cs3 | cs4 | cs5 | cs6 | cs7 | cs8 | cs14 | cs17 | cs21 | cf nd |
| EatA+ | 17 | 12 | 7 | 19 | 3 | 18 | 25 | 15 | 2 | 2 | 9 | 14 | 7 |
| EtpA+ | 17 | 12 | 8 | 21 | 1 | 0 | 3 | 14 | 0 | 12 | 9 | 14 | 16 |
| EatA+(%) | 0.80952381 | 0.86 | 0.7 | 0.76 | 0.43 | 0.78 | 0.64 | 0.938 | 1 | 0.14286 | 0.9 | 0.82353 | 0.19444444 |
| EtpA+(%) | 0.80952381 | 0.86 | 0.8 | 0.84 | 0.14 | 0 | 0.08 | 0.875 | 0 | 0.85714 | 0.9 | 0.82353 | 0.44444444 |
| either + | 18 | 13 | 9 | 23 | 3 | 18 | 26 | 15 | 2 | 13 | 9 | 16 | 18 |
| either + (%) | 86 | 93 | 90 | 92 | 43 | 78 | 67 | 94 | 100 | 93 | 90 | 94 | 50 |

TABLE 8
YghJ prevalence.

| | | Strain | | | | | | | | | |
|---|---|---|---|---|---|---|---|---|---|---|---|
| Stock # | YghJ | designation | EatA | EtpA | CFs | LT | ST | Severity | Symptom | CFA/I | CS1 |
| 350 | 1 | ETEC | 1 | 1 | | 1 | 1 | 2 | D | | |
| | | ThroopD “1” | | | | | | | | | |
| 1388 | 1 | ETEC | 1 | 0 | 1 | 1 | 1 | 2 | D | | |
| | | Envira 8/11 | | | | | | | | | |
| 1389 | 1 | ETEC | 1 | 0 | 1 | 1 | 1 | 2 | D | | |
| | | Envira 10/1 | | | | | | | | | |
| 1390 | 1 | ETEC Jurua | 1 | 1 | 1 | 1 | 0 | 2 | D | | |
| | | 18/11 | | | | | | | | | |
| 1391 | 1 | ETEC Jurua | 1 | 1 | 1 | 1 | 0 | 2 | D | | |
| | | 20/10 | | | | | | | | | |
| 2007 | 1 | P02997067.1 | 1 | 0 | | 1 | 1 | 2 | D | | |
| 2021 | 0 | MP021552.1 | 1 | 0 | | 1 | 1 | 2 | D | | |
| 2036 | 1 | MP021561.1 | 1 | 0 | | 0 | 1 | 2 | D | | |
| 2053 | 0 | MP021017.1 | 0 | 0 | | 0 | 1 | 2 | D | | |
| 2063 | 1 | MP020980.1 | 1 | 0 | | 0 | 1 | 2 | D | | |
| 2097 | 1 | 2720900 | 1 | 1 | 1 | 0 | 1 | 1 | D | CFA/I | |
| 2098 | 1 | 2719100 | 1 | 1 | 1 | 0 | 1 | 1 | D | CFA/I | |
| 2099 | 0 | 2722950 | 1 | 1 | 1 | 1 | 1 | 1 | D | CFA/I | |
| 2100 | 1 | 2726800 | 0 | 1 | 1 | 1 | 1 | 1 | D | | |
| 2101 | 0 | 2726950 | 1 | 1 | 1 | 0 | 1 | 1 | D | | |
| 2102 | 1 | 2729250 | 1 | 0 | 1 | 1 | 1 | 1 | D | CFA/I | |
| 2103 | 1 | 2747800 | 1 | 0 | 1 | 1 | 1 | 2 | D | | |
| 2104 | 1 | 2741950 | 1 | 1 | 1 | 1 | 1 | 2 | D | | CS1 |
| 2105 | 1 | 2735000 | 1 | 1 | 1 | 1 | 0 | 2 | D | | |
| 2106 | 1 | 2730350 | 0 | 1 | 1 | 1 | 1 | 2 | D | CFA/I | |
| 2107 | 1 | 2730450 | 1 | 1 | 1 | 1 | 1 | 2 | D | | CS1 |
| 2108 | 1 | 2770900 | 1 | 0 | 1 | 1 | 1 | 2 | D | | |
| 2109 | 1 | 2780750 | 0 | 0 | 0 | 0 | 1 | 2 | D | | |
| 2110 | 1 | 2731150 | 1 | 0 | 1 | 1 | 0 | 2 | D | | |
| 2111 | 1 | 2785200 | 0 | 0 | 0 | 1 | 0 | 2 | D | | |
| 2112 | 1 | 2733950 | 1 | 0 | 1 | 1 | 1 | 2 | D | | |
| 2113 | 1 | 2749250 | 1 | 0 | 1 | 1 | 1 | 2 | D | | |
| 2114 | 1 | 2756500 | 1 | 1 | 1 | 1 | 0 | 2 | D | | |
| 2115 | 1 | 2788150 | 0 | 0 | 0 | 1 | 1 | 2 | D | | |
| 2116 | 1 | 2762100 | 1 | 0 | 1 | 1 | 0 | 2 | D | | |
| 2117 | 1 | 2845350 | 0 | 0 | 1 | 1 | 1 | 1 | D | | |
| 2118 | 1 | 2845650 | 0 | 0 | 1 | 1 | 1 | 1 | D | | |
| 2119 | 1 | 2846750 | 1 | 1 | 1 | 1 | 1 | 1 | D | | |
| 2120 | 0 | 2848050 | 0 | 0 | 1 | 1 | 1 | 1 | D | | |
| 2121 | 1 | 2850400 | 1 | 1 | 1 | 1 | 0 | 2 | D | | |
| 2122 | 1 | 2850750 | 1 | 1 | 1 | 1 | 0 | 2 | D | | |
| 2123 | 1 | 2851500 | 1 | 1 | 1 | 1 | 0 | 1 | D | | |
| 2124 | 1 | 2853500 | 1 | 1 | 0 | 1 | 0 | 2 | D | | |
| 2125 | 1 | 2854350 | 0 | 1 | 1 | 1 | 0 | 2 | D | | |
| 2126 | 1 | 2860050 | 1 | 0 | 1 | 1 | 1 | 2 | D | | |
| 2127 | 1 | 2860650 | 1 | 0 | 1 | 1 | 1 | 2 | D | | |
| 2128 | 0 | 2861200 | 0 | 0 | 1 | 0 | 1 | 2 | D | | |
| 2129 | 1 | 2862600 | 0 | 0 | 1 | 0 | 1 | 2 | D | | |
| 2130 | 0 | 2863500 | 0 | 0 | 1 | 0 | 1 | 2 | D | CFA/I | |
| 2131 | 1 | 2864350 | 0 | 0 | 0 | 1 | 1 | 2 | D | | |
| 2132 | 1 | 2865200 | 0 | 0 | 0 | 0 | 1 | 2 | D | | |
| 2133 | 1 | 2866350 | 1 | 0 | 1 | 1 | 1 | 2 | D | | |
| 2134 | 1 | 2866450 | 1 | 1 | 1 | 1 | 0 | 2 | D | | |
| 2135 | 1 | 2866550 | 1 | 1 | 1 | 1 | 0 | 2 | D | | |
| 2136 | 1 | 2866750 | 1 | 1 | 1 | 1 | 0 | 2 | D | | |
| 2137 | 1 | 2867750 | 1 | 1 | 1 | 1 | 0 | 2 | D | | |
| 2138 | 1 | 2871950 | 1 | 1 | 1 | 1 | 0 | 2 | D | | |
| 2139 | 1 | 2872000 | 1 | 1 | 1 | 1 | 0 | 2 | D | | |
| 2140 | 1 | 2872800 | 1 | 1 | 1 | 1 | 0 | 2 | D | | |
| 2141 | 1 | 2875000 | 1 | 1 | 1 | 1 | 0 | 2 | D | | |
| 2142 | 1 | 2875150 | 0 | 0 | 1 | 1 | 1 | 2 | D | | |
| 2143 | 1 | 199900.1 | 1 | 1 | 1 | 1 | 0 | 2 | D | | |
| 2144 | 1 | 199900.2 | 1 | 1 | 1 | 1 | 0 | 2 | D | | |
| 2145 | 1 | 21600.1 | 0 | 1 | 1 | 1 | 1 | 2 | D | | |
| 2146 | 1 | 21600.2 | 0 | 1 | 1 | 1 | 1 | 2 | D | | |
| 2147 | 1 | 21600.3 | 0 | 1 | 1 | 1 | 1 | 2 | D | | |
| 2148 | 1 | 21600.4 | 0 | 1 | 1 | 1 | 1 | 2 | D | | |
| 2149 | 1 | BCE006, | 1 | 1 | 1 | 1 | 1 | 0 | A | | CS1 |
| | | MS023 | | | | | | | | | |
| 2150 | 0 | BCE011, | 0 | 0 | 1 | 0 | 1 | 0 | A | CFA/1 | |
| | | MS001 | | | | | | | | | |
| 2151 | 1 | BCE019, | 1 | 1 | 1 | 1 | 1 | 0 | A | | CS1 |
| | | MS013 | | | | | | | | | |
| 2152 | 0 | BCE030, | 1 | 1 | 1 | 0 | 1 | 0 | A | CFA/I | |
| | | MS009 | | | | | | | | | |
| 2153 | 1 | BCE032, | 1 | 1 | 1 | 1 | 1 | 0 | A | | CS1 |
| | | MS012 | | | | | | | | | |
| 2154 | 1 | BCE034, | 1 | 1 | 1 | 0 | 1 | 0 | A | CFA/1 | |
| | | MS014 | | | | | | | | | |
| 2155 | 1 | P0304816.1 | 1 | 0 | | 0 | 1 | 2 | D | | |
| 2170 | 1 | P0305260.1 | 0 | 1 | | 0 | 1 | 2 | D | | |
| 2184 | 1 | P0305293.1 | 1 | 0 | | 1 | 1 | 2 | D | | |
| 2199 | 1 | p0301867.1 | 1 | 1 | | 0 | 1 | 2 | D | | |
| 2208 | 1 | P0302293.2 | 0 | 1 | | 1 | 1 | 2 | D | | |
| 2216 | 1 | P0302308.1 | 0 | 1 | | 1 | 1 | 2 | D | | |
| 2226 | 1 | P0304777.1 | 1 | 1 | | 1 | 0 | 2 | D | | |
| 2267 | 1 | MP020940.1 | 0 | 0 | | 0 | 1 | 2 | D | | |
| 2282 | 1 | P0298942.1 | 1 | 0 | | 1 | 1 | 2 | D | | |
| 2295 | 1 | P0299438.2 | 0 | 1 | | 0 | 1 | 2 | D | | |
| 2305 | 1 | P0299917.1 | 0 | 0 | | 1 | 1 | 2 | D | | |
| 2315 | 1 | ETP050002 | 1 | 1 | 1 | 0 | 1 | 2 | D | CFA/I | |
| 2316 | 1 | ETP050003 | 0 | 0 | 0 | 0 | 1 | 2 | D | | |
| 2317 | 1 | ETP050007 | 0 | 0 | 1 | 1 | 1 | 2 | D | | |
| 2318 | 1 | ETP050008 | 0 | 1 | 0 | 0 | 1 | 2 | D | | |
| 2319 | 1 | ETP050009 | 0 | 0 | 1 | 1 | 1 | 2 | D | | |
| 2320 | 1 | ETP050010 | 0 | 1 | 1 | 1 | 1 | 2 | D | | |
| 2321 | 1 | ETP050011 | 0 | 1 | 1 | 1 | 1 | 2 | D | | |
| 2322 | 1 | ETP050012 | 0 | 0 | 1 | 0 | 1 | 2 | D | | |
| 2323 | 1 | ETP050015 | 0 | 0 | 1 | 1 | 1 | 2 | D | | |
| 2324 | 1 | ETP050016 | 0 | 1 | 0 | 0 | 1 | 2 | D | | |
| 2325 | 1 | ETP050017 | 1 | 0 | 1 | 1 | 1 | 2 | D | | |
| 2326 | 1 | ETP050019 | 0 | 1 | 0 | 1 | 1 | 2 | D | | |
| 2327 | 1 | ETP05020 | 0 | 0 | 1 | 1 | 0 | 2 | D | | |
| 2328 | 1 | ETP05026 | 1 | 1 | 1 | 1 | 1 | 2 | D | CFA/I | |
| 2329 | 0 | ETP050035 | 0 | 0 | 0 | 1 | 1 | 2 | D | | |
| 2330 | 1 | ETP050038 | 0 | 0 | 1 | 1 | 1 | 2 | D | | |
| 2331 | 1 | ETP050039 | 0 | 1 | 1 | 1 | 1 | 2 | D | | |
| 2332 | 0 | ETP050044 | 0 | 0 | 1 | 0 | 1 | 2 | D | CFA/I | |
| 2333 | 1 | ETP050046 | 1 | 1 | 1 | 0 | 1 | 2 | D | CFA/I | |
| 2334 | 1 | ETP050047 | 0 | 0 | 0 | 1 | 1 | 2 | D | | |
| 2335 | 1 | ETP050050 | 0 | 0 | 1 | 1 | 0 | 2 | D | | |
| 2336 | 1 | 174750 | 1 | 0 | 1 | 1 | 1 | 2 | D | | CS1 |
| 2337 | 1 | 174900 | 0 | 0 | 0 | 0 | 1 | 2 | D | | |
| 2338 | 1 | 178200 | 0 | 0 | 0 | 0 | 1 | 2 | D | | |
| 2339 | 0 | 178850 | 1 | 0 | 0 | 1 | 1 | 2 | D | | |
| 2340 | 1 | 178900 | 1 | 1 | 1 | 0 | 1 | 2 | D | CFA/I | |
| 2341 | 1 | 179100 | 0 | 0 | 0 | 1 | 1 | 2 | D | | |
| 2342 | 1 | 179550 | 1 | 1 | 1 | 1 | 0 | 2 | D | | |
| 2343 | 1 | 180200 | 1 | 1 | 1 | 1 | 1 | 2 | D | | |
| 2344 | 1 | 180600 | 1 | 1 | 1 | 1 | 1 | 2 | D | | |
| 2345 | 1 | 180050 | 1 | 1 | 1 | 1 | 0 | 2 | D | | |
| 2346 | 0 | C034666 | 1 | 0 | 0 | 1 | 1 | 2 | D | | |
| 2347 | 1 | C035134 | 1 | 1 | 1 | 1 | 1 | 2 | D | | |
| 2348 | 1 | C035209 | 0 | 0 | 1 | 0 | 1 | 2 | D | | |
| 2349 | 1 | C035213 | 1 | 1 | 1 | 1 | 1 | 2 | D | | |
| 2350 | 1 | C035605 | 1 | 1 | 1 | 1 | 0 | 2 | D | | |
| 2351 | 1 | C035662 | 0 | 0 | 0 | 1 | 1 | 2 | D | | |
| 2352 | 1 | C035776 | 1 | 0 | 1 | 0 | 1 | 2 | D | | |
| 2353 | 1 | C035959 | 0 | 1 | 1 | 1 | 1 | 2 | D | | |
| 2354 | 1 | C036255 | 0 | 1 | 1 | 1 | 1 | 2 | D | | |
| 2355 | 1 | 173150 | 0 | 0 | 1 | 1 | 1 | 2 | D | | CS1 |
| 2356 | 1 | BCE001, | 0 | 1 | 0 | 1 | 0 | 0 | A | | |
| | | MS16 | | | | | | | | | |
| 2357 | 1 | BCE002, | 0 | 1 | 1 | 1 | 1 | 0 | A | | CS1 |
| | | MS12 | | | | | | | | | |
| 2358 | | BCE005, | 0 | 1 | 0 | 1 | 1 | 0 | A | | |
| | | MS23 | | | | | | | | | |
| 2359 | 1 | BCE007, | 1 | 1 | 1 | 1 | 1 | 0 | A | | CS1 |
| | | MS11 | | | | | | | | | |
| 2360 | 1 | BCE008, | 0 | 0 | 0 | 1 | 0 | 0 | A | | |
| | | MS1 | | | | | | | | | |
| 2361 | 0 | BCE008, | 0 | 0 | 1 | 0 | 1 | 0 | A | | |
| | | MS13 | | | | | | | | | |
| 2362 | 1 | BCE019, | 1 | 1 | 1 | 1 | 1 | 0 | A | | |
| | | MS16 | | | | | | | | | |
| 2363 | 1 | BCE035, | 1 | 0 | 1 | 1 | 0 | 0 | A | | |
| | | MS8 | | | | | | | | | |
| 2364 | 1 | BCE039, | 0 | 0 | 0 | 1 | 0 | 0 | A | | |
| | | MS13 | | | | | | | | | |
| 2365 | 0 | BCE041, | 0 | 0 | 0 | 1 | 0 | 0 | A | | |
| | | MS11 | | | | | | | | | |
| 2366 | 1 | BCE046, | 0 | 0 | 0 | 1 | 0 | 0 | A | | |
| | | MS16 | | | | | | | | | |
| 2367 | 1 | BCE049, | 1 | 0 | 1 | 1 | 1 | 0 | A | | |
| | | MS9 | | | | | | | | | |
| 2368 | 1 | BCE054, | 0 | 0 | 1 | 1 | 1 | 0 | A | | |
| | | MS24 | | | | | | | | | |
| 2369 | 0 | BCE058, | 1 | 1 | 1 | 0 | 1 | 0 | A | CFA/I | |
| | | MS13 | | | | | | | | | |
| 2370 | 1 | BCE062, | 1 | 1 | 1 | 1 | 1 | 0 | A | | |
| | | MS24 | | | | | | | | | |
| 2371 | 1 | BCE063, | 0 | 1 | 0 | 1 | 0 | 0 | A | | |
| | | MS14 | | | | | | | | | |
| 2372 | 1 | BCE068, | 0 | 1 | 1 | 1 | 1 | 0 | A | | |
| | | MS10 | | | | | | | | | |
| 2373 | 1 | BCE068, | 0 | 1 | 0 | 0 | 1 | 0 | A | | |
| | | MS23 | | | | | | | | | |
| 2374 | 1 | BCE069, | 1 | 1 | 1 | 1 | 1 | 0 | A | CFA/I | |
| | | MS15 | | | | | | | | | |
| 2375 | 1 | BCE069, | 1 | 1 | 1 | 1 | 1 | 0 | A | | CS1 |
| | | MS9 | | | | | | | | | |
| 2381 | 1 | mp021566.1 | 1 | 1 | | 1 | 1 | 2 | D | | |
| 2409 | 1 | ETP98109 | 1 | 1 | 1 | 0 | 1 | 2 | D | CFA/I | |
| 2410 | 1 | ETP98111 | 1 | 1 | 1 | 1 | 1 | 2 | D | | |
| 2411 | 1 | ETP98112 | 1 | 1 | 1 | 1 | 1 | 2 | D | | CS1 |
| 2412 | 1 | ETP98114 | 0 | 1 | 1 | 0 | 1 | 2 | D | | |
| 2413 | 1 | ETP98115 | 0 | 1 | 1 | 1 | 1 | 2 | D | | |
| 2414 | 1 | ETP98068 | 1 | 1 | 1 | 1 | 1 | 2 | D | CFA/I | |
| 2415 | 1 | ETP98073 | 1 | 0 | 1 | 1 | 1 | 2 | D | | |
| 2416 | 1 | ETP98097 | 1 | 1 | 1 | 1 | 1 | 2 | D | | |
| 2417 | 1 | ETP98103 | 1 | 1 | 1 | 1 | 1 | 2 | D | | CS1 |
| 2418 | 1 | ETP98105 | 1 | 1 | 1 | 1 | 1 | 2 | D | | CS1 |
| 2419 | 1 | ETP98053 | 1 | 0 | 1 | 1 | 1 | 2 | D | | |
| 2420 | 0 | ETP98056 | 1 | 0 | 1 | 0 | 1 | 2 | D | | |
| 2421 | 1 | ETP98061 | 1 | 1 | 1 | 0 | 1 | 2 | D | | |
| 2422 | 0 | ETP98062 | 1 | 0 | 1 | 0 | 1 | 2 | D | | |
| 2423 | 0 | ETP98066 | 0 | 0 | 1 | 1 | 1 | 2 | D | | |
| 2424 | 0 | ETP98004 | 0 | 0 | 0 | 0 | 1 | 2 | D | | |
| 2425 | 1 | ETP98015 | 1 | 0 | 1 | 0 | 1 | 2 | D | | |
| 2426 | 1 | ETP98028 | 1 | 0 | 1 | 0 | 1 | 2 | D | | |
| 2427 | 1 | ETP98038 | 1 | 1 | 1 | 0 | 1 | 2 | D | CFA/I | |
| 2428 | 1 | ETP98042 | 1 | 0 | 1 | 1 | 1 | 2 | D | | |
| 2430 | 1 | BCE003, | 1 | 1 | 0 | 1 | 1 | 1 | D | | |
| | | DS5 | | | | | | | | | |
| 2431 | 1 | BCE021, | 0 | 1 | 0 | 0 | 1 | 1 | D | | |
| | | DS7 | | | | | | | | | |
| 2432 | 1 | BCE046, | 1 | 0 | 1 | 1 | 1 | 1 | D | | |
| | | DS7 | | | | | | | | | |
| 2433 | 1 | BCE062, | 1 | 1 | 0 | 1 | 1 | 1 | D | | |
| | | DS2 | | | | | | | | | |
| 2434 | 1 | BCE007, | 1 | 0 | 1 | 1 | 1 | 1 | D | | |
| | | DS5 | | | | | | | | | |
| 2435 | 1 | BCE022, DS6 | 1 | 1 | 1 | 0 | 1 | 1 | D | CFA/I | |
| 2436 | 1 | BCE049, | 1 | 1 | 0 | 1 | 1 | 1 | D | | |
| | | DS3 | | | | | | | | | |
| 2437 | 1 | BCE063, | 0 | 1 | 1 | 1 | 1 | 1 | D | | |
| | | DS4 | | | | | | | | | |
| 2438 | 1 | BCE011, | 0 | 1 | 0 | 1 | 0 | 1 | D | | |
| | | DS3 | | | | | | | | | |
| 2439 | 1 | BCE035, | 1 | 1 | 1 | 1 | 1 | 1 | D | | |
| | | DS6 | | | | | | | | | |
| 2440 | 1 | BCE054, | 1 | 0 | 1 | 1 | 1 | 1 | D | | |
| | | DS4 | | | | | | | | | |
| 2441 | 1 | BCE066, | 0 | 1 | 0 | 1 | 0 | 1 | D | | |
| | | DS5 | | | | | | | | | |
| 2442 | 1 | BCE013, | 1 | 1 | 1 | 0 | 1 | 1 | D | CFA/I | |
| | | DS1 | | | | | | | | | |
| 2443 | 1 | BCE039, | 1 | 0 | 1 | 1 | 1 | 1 | D | | |
| | | DS2 | | | | | | | | | |
| 2444 | 1 | BCE055, | 1 | 1 | 1 | 1 | 0 | 1 | D | | |
| | | DS1 | | | | | | | | | |
| 2445 | 1 | BCE069, | 1 | 1 | 1 | 1 | 1 | 1 | D | | CS1 |
| | | DS2 | | | | | | | | | |
| 2446 | 1 | BCE018, | 1 | 1 | 0 | 1 | 1 | 1 | D | | |
| | | DS6 | | | | | | | | | |
| 2447 | 1 | BCE046, | 0 | 1 | 0 | 1 | 0 | 1 | D | | |
| | | DS2 | | | | | | | | | |
| 2448 | 1 | BCE061, | 0 | 0 | 0 | 1 | 1 | 1 | D | | |
| | | DS1 | | | | | | | | | |
| 2449 | 1 | BCE129, | 0 | 1 | | 0 | 1 | 1 | D | | |
| | | DS2 | | | | | | | | | |
| totals | 161 | | 106 | 102 | 126 | 134 | 142 | | | | |

| Stock # | CS2 | CS3 | CS4 | CS5 | CS6 | CS7 | CS8 | CS14 | CS17 | CS21 | CF0ve |
|---|---|---|---|---|---|---|---|---|---|---|---|
| 350 | | | | | | | | | | | |
| 1388 | | | | CS5 | CS6 | | | | | | |
| 1389 | | | | CS5 | CS6 | | | | | | |
| 1390 | | | | | | CS7 | | | | | |
| 1391 | | | | | | CS7 | | | | | |
| 2007 | | | | | | | | | | | |
| 2021 | | | | | | | | | | | |
| 2036 | | | | | | | | | | | |
| 2053 | | | | | | | | | | | |
| 2063 | | | | | | | | | | | |
| 2097 | | | | | | | | | | | |
| 2098 | | | | | | | | | | CS21 | |
| 2099 | | | | | | | | | | | |
| 2100 | | | | | | | | CS14 | | | |
| 2101 | | | | | | | | | | CS21 | |
| 2102 | | | | | | | | | | | |
| 2103 | | | | CS5 | CS6 | | | | | | |
| 2104 | | CS3 | | | | | | | | CS21 | |
| 2105 | | | | | | CS7 | | | | | |
| 2106 | | | | | | | | | | CS21 | |
| 2107 | | CS3 | | | | | | | | CS21 | |
| 2108 | | | | CS5 | CS6 | | | | | | |
| 2109 | | | | | | | | | | | 1 |
| 2110 | | | | | CS6 | | CS8 | | | | |
| 2111 | | | | | | | | | | | 1 |
| 2112 | | | CS4 | | CS6 | | | | | | |
| 2113 | | | | CS5 | CS6 | | | | | | |
| 2114 | | | | | | CS7 | | | | | |
| 2115 | | | | | | | | | | | 1 |
| 2116 | | | | | CS6 | | CS8 | | | | |
| 2117 | | | CS4 | | CS6 | | | | | | |
| 2118 | | | CS4 | | CS6 | | | | | | |
| 2119 | CS2 | CS3 | | | | | | | | | |
| 2120 | | | | CS5 | CS6 | | | | | | |
| 2121 | | | | | | CS7 | | | | | |
| 2122 | | | | | | | | | CS17 | | |
| 2123 | | | | | | CS7 | | | | | |
| 2124 | | | | | | | | | | | 1 |
| 2125 | | | | | | | | CS14 | | | |
| 2126 | | | | CS5 | CS6 | | | | | | |
| 2127 | | | | CS5 | CS6 | | | | | | |
| 2128 | | | | | CS6 | | | | | | |
| 2129 | | | CS4 | | CS6 | | | | | | |
| 2130 | | | | | | | | | | | |
| 2131 | | | | | | | | | | | 1 |
| 2132 | | | | | | | | | | | 1 |
| 2133 | | | | CS5 | CS6 | | | | | | |
| 2134 | | | | | | CS7 | | | | | |
| 2135 | | | | | | CS7 | | | | | |
| 2136 | | | | | | CS7 | | | | | |
| 2137 | | | | | | CS7 | | | | | |
| 2138 | CS2 | CS3 | | | | | | | | | |
| 2139 | CS2 | CS3 | | | | | | | | | |
| 2140 | | | | | | CS7 | | | | | |
| 2141 | | | | | | CS7 | | | | | |
| 2142 | | | CS4 | | CS6 | | | | | | |
| 2143 | | | | | | | | | CS17 | | |
| 2144 | | | | | | | | | CS17 | | |
| 2145 | | | | | | | | CS14 | | | |
| 2146 | | | | | | | | CS14 | | | |
| 2147 | | | | | | | | CS14 | | | |
| 2148 | | | | | | | | CS14 | | | |
| 2149 | | CS3 | | | | | | | | | |
| 2150 | | | | | | | | | | | |
| 2151 | | CS3 | | | | | | | | | |
| 2152 | | | | | | | | | | | |
| 2153 | | CS3 | | | | | | | | | |
| 2154 | | | | | | | | | | | |
| 2155 | | | | | | | | | | | |
| 2170 | | | | | | | | | | | |
| 2184 | | | | | | | | | | | |
| 2199 | | | | | | | | | | | |
| 2208 | | | | | | | | | | | |
| 2216 | | | | | | | | | | | |
| 2226 | | | | | | | | | | | |
| 2267 | | | | | | | | | | | |
| 2282 | | | | | | | | | | | |
| 2295 | | | | | | | | | | | |
| 2305 | | | | | | | | | | | |
| 2315 | | | | | | | | | | | |
| 2316 | | | | | | | | | | | 1 |
| 2317 | | | | CS5 | CS6 | | | | | | |
| 2318 | | | | | | | | | | | 1 |
| 2319 | | | | CS5 | CS6 | | | | | | |
| 2320 | | CS3 | | | | | | | | CS21 | |
| 2321 | CS2 | CS3 | | | | | | | | | |
| 2322 | | | | | CS6 | | | | | | |
| 2323 | | | | CS5 | CS6 | | | | | | |
| 2324 | | | | | | | | | | | 1 |
| 2325 | | | | CS5 | CS6 | | | | | | |
| 2326 | | | | | | | | | | | 1 |
| 2327 | | | | | | CS7 | | | | | |
| 2328 | | | | | | | | | | | |
| 2329 | | | | | | | | | | | 1 |
| 2330 | | | | CS5 | CS6 | | | | | | |
| 2331 | | | | | | | | CS14 | | | |
| 2332 | | | | | | | | | | | |
| 2333 | | | | | | | | | | CS21 | |
| 2334 | | | | | | | | | | | 1 |
| 2335 | | | | | | | | | CS17 | | |
| 2336 | | CS3 | | | | | | | | CS21 | |
| 2337 | | | | | | | | | | | 1 |
| 2338 | | | | | | | | | | | 1 |
| 2339 | | | | | | | | | | | 1 |
| 2340 | | | | | | | | | | CS21 | |
| 2341 | | | | | | | | | | | 1 |
| 2342 | | | | | | CS7 | | | | | |
| 2343 | | | | | | CS7 | | | | | |
| 2344 | | | | | | | | | CS17 | | |
| 2345 | | | | | | | | | CS17 | | |
| 2346 | | | | | | | | | | | 1 |
| 2347 | | | | | | | | | CS17 | | |
| 2348 | CS2 | CS3 | | | | | | | | | |
| 2349 | | | | | | | | | CS17 | | |
| 2350 | | | | | | | | | CS17 | | |
| 2351 | | | | | | | | | | | 1 |
| 2352 | | | CS4 | | CS6 | | | | | | |
| 2353 | | | | | | | | CS14 | | | |
| 2354 | | | | | | | | CS14 | | | |
| 2355 | | CS3 | | | | | | | | CS21 | |
| 2356 | | | | | | | | | | | 1 |
| 2357 | | CS3 | | | | | | | | | |
| 2358 | | | | | | | | | | | 1 |
| 2359 | | CS3 | | | | | | | | CS21 | |
| 2360 | | | | | | | | | | | 1 |
| 2361 | | | | | CS6 | | | | | | |
| 2362 | CS2 | CS3 | | | | | | | | | |
| 2363 | | | | | | CS7 | | | | | |
| 2364 | | | | | | | | | | | 1 |
| 2365 | | | | | | | | | | | 1 |
| 2366 | | | | | | | | | | | 1 |
| 2367 | CS2 | CS3 | | | | | | | | CS21 | |
| 2368 | | | | | | | | CS14 | | | |
| 2369 | | | | | | | | | | | |
| 2370 | CS2 | CS3 | | | | | | | | | |
| 2371 | | | | | | | | | | | 1 |
| 2372 | CS2 | CS3 | | | | | | | | | |
| 2373 | | | | | | | | | | | 1 |
| 2374 | | | | | | | | | | | |
| 2375 | | CS3 | | | | | | | | CS21 | |
| 2381 | | | | | | | | | | | |
| 2409 | | | | | | | | | | | |
| 2410 | | | CS4 | | CS6 | | | | | | |
| 2411 | | CS3 | | | | | | | | CS21 | |
| 2412 | | | | | | | | CS14 | | | |
| 2413 | | | | | CS6 | | | | | | |
| 2414 | | | | | | | | | | | |
| 2415 | | | | CS5 | CS6 | | | | | | |
| 2416 | | | | | | | | CS14 | | | |
| 2417 | | CS3 | | | | | | | | CS21 | |
| 2418 | | CS3 | | | | | | | | CS21 | |
| 2419 | | | | CS5 | CS6 | | | | | | |
| 2420 | | | | | CS6 | | | | | | |
| 2421 | | | | | CS6 | | | | | | |
| 2422 | | | | | | | | CS14 | | | |
| 2423 | | | | | CS6 | | | | | | |
| 2424 | | | | | | | | | | | 1 |
| 2425 | | | | CS5 | CS6 | | | | | | |
| 2426 | | | | CS5 | CS6 | | | | | | |
| 2427 | | | | | | | | | | | |
| 2428 | | | | CS5 | CS6 | | | | | | |
| 2430 | | | | | | | | | | | 1 |
| 2431 | | | | | | | | | | | 1 |
| 2432 | | | | CS5 | CS6 | | | | | | |
| 2433 | | | | | | | | | | | 1 |
| 2434 | | | | CS5 | CS6 | | | | | | |
| 2435 | | | | | | | | | | | |
| 2436 | | | | | | | | | | | 1 |
| 2437 | | | | | | | | CS14 | | | |
| 2438 | | | | | | | | | | | 1 |
| 2439 | CS2 | CS3 | | | | | | | | | |
| 2440 | | | | CS5 | CS6 | | | | | | |
| 2441 | | | | | | | | | | | 1 |
| 2442 | | | | | | | | | | | |
| 2443 | | | | CS5 | CS6 | | | | | | |
| 2444 | | | | | | | | | CS17 | | |
| 2445 | | CS3 | | | | | | | | CS21 | |
| 2446 | | | | | | | | | | | 1 |
| 2447 | | | | | | | | | | | 1 |
| 2448 | | | | | | | | | | | 1 |
| 2449 | | | | | | | | | | | |
| totals | | | | | | | | | | | 36 |

Similar data as Table 6 showing presence (1 = yes), or absence (0 = no) of YghJ in culture supernatant as determined by immunoblotting (column B).

TABLE 9
Presence of eaeH gene in isolates sequenced at Genome Sequencing Center for
Infectious Diseases (GSCID).

| | | Strain | | | | |
|---|---|---|---|---|---|---|
| Stock # | EaeH | designation | GENOTYPE | SOURCE | COMMENTS | severity |
| 2150 | 1 | BCE011, MS- | asymptomatic | icddr, b | CFA/1 ST+ 2002 asymptomatic colonization | 0 |
| | | 01 | colonization | | | |
| 2154 | 1 | BCE034, MS- | asymptomatic | icddr, b | CFA/1 ST+ 2003 asymptomatic colonization | 0 |
| | | 14 | colonization | | | |
| 2170 | 1 | P0305260.1 | clinical E. coli | icddr, b | tox-, severe cholera-like disease, hospitalized patient, | 2 |
| | | | isolate | | isolate 1 | |
| 1388 | 1 | ETEC Envira | clinical ETEC isolate | from Brazil | CS5/CS6; LT; ST; severe cholera-like disease from 1998 | 2 |
| | | 8/11 | | | | |
| 1389 | 1 | ETEC Envira | clinical ETEC isolate | from Brazil | CS5/CS6; LT; ST; severe cholera-like disease from 1998 | 2 |
| | | 10/1 | | | | |
| 2097 | 1 | 2720900 | clinical ETEC isolate | icddr, b | CFA/I; ST; mild, self-limited diarrhea; isolate from 2007 | 1 |
| 2099 | 1 | 2722950 | clinical ETEC isolate | icddr, b | CFA/I LT/ST 2007 mild, self-limited diarrhea | 1 |
| 2100 | 1 | 2726800 | clinical ETEC isolate | icddr, b | CS14 LT/ST 2007 mild, self-limited diarrhea | 1 |
| 2101 | 1 | 2726950 | clinical ETEC isolate | icddr, b | CS21 ST 2007 mild, self-limited diarrhea | 1 |
| 2102 | 1 | 2729250 | clinical ETEC isolate | icddr, b | CFA/I LT/ST 2007 mild, self-limited diarrhea | 1 |
| 2103 | 1 | 2747800 | clinical ETEC isolate | icddr, b | CS5 + CS6 LT/ST 2007 severe cholera-like disease, | 2 |
| | | | | | hospitalized patient | |
| 2105 | 1 | 2735000 | clinical ETEC isolate | icddr, b | CS7 LT 2007 severe cholera-like disease, hospitalized | 2 |
| | | | | | patient | |
| 2106 | 1 | 2730350 | clinical ETEC isolate | icddr, b | CFA/I + CS21 LT/ST 2007 severe cholera-like disease, | 2 |
| | | | | | hospitalized patient | |
| 2108 | 1 | 2770900 | clinical ETEC isolate | icddr, b | CS5 + CS6 LT/ST 2007 severe cholera-like disease, | 2 |
| | | | | | hospitalized patient | |
| 2109 | 1 | 2780750 | clinical ETEC isolate | icddr, b | CF-ve ST 2007 severe cholera-like disease, hospitalized | 2 |
| | | | | | patient | |
| 2110 | 1 | 2731150 | clinical ETEC isolate | icddr, b | CS6 + CS8 LT 2007 severe cholera-like disease, hospitalized | 2 |
| | | | | | patient | |
| 2111 | 1 | 2785200 | clinical ETEC isolate | icddr, b | CF-ve LT 2007 severe cholera-like disease, hospitalized | 2 |
| | | | | | patient | |
| 2112 | 1 | 2733950 | clinical ETEC isolate | icddr, b | CS4 + CS6 LT/ST 2007 severe cholera-like disease, | 2 |
| | | | | | hospitalized patient | |
| 2113 | 1 | 2749250 | clinical ETEC isolate | icddr, b | CS5 + CS6 LT/ST 2007 severe cholera-like disease, | 2 |
| | | | | | hospitalized patient | |
| 2114 | 1 | 2756500 | clinical ETEC isolate | icddr, b | CS7 LT 2007 severe cholera-like disease, hospitalized | 2 |
| | | | | | patient | |
| 2115 | 1 | 2788150 | clinical ETEC isolate | icddr, b | CF-ve LT/ST 2007 severe cholera-like disease, hospitalized | 2 |
| | | | | | patient | |
| 2116 | 1 | 2762100 | clinical ETEC isolate | icddr, b | CS6 + CS8 LT 2007 severe cholera-like disease, hospitalized | 2 |
| | | | | | patient | |
| 2117 | 1 | 2845350 | clinical ETEC isolate | icddr, b | CS4 + CS6 LT/ST 2008 mild, self-limited diarrhea | 1 |
| 2118 | 1 | 2845650 | clinical ETEC isolate | icddr, b | CS4 + CS6 LT/ST 2008 mild, self-limited diarrhea | 1 |
| 2120 | 1 | 2848050 | clinical ETEC isolate | icddr, b | CS5 + CS6 LT/ST 2008 mild, self-limited diarrhea | 1 |
| 2121 | 1 | 2850400 | clinical ETEC isolate | icddr, b | CS7 LT 2008 severe cholera-like disease, hospitalized | 2 |
| | | | | | patient | |
| 2122 | 1 | 2850750 | clinical ETEC isolate | icddr, b | CS17 LT 2008 severe cholera-like disease, hospitalized | 2 |
| | | | | | patient | |
| 2124 | 1 | 2853500 | clinical ETEC isolate | icddr, b | CF-ve LT 2008 severe cholera-like disease, hospitalized | 2 |
| | | | | | patient | |
| 2125 | 1 | 2854350 | clinical ETEC isolate | icddr, b | CS14 LT 2008 severe cholera-like disease, hospitalized | 2 |
| | | | | | patient | |
| 2126 | 1 | 2860050 | clinical ETEC isolate | icddr, b | CS5 + CS6 LT/ST 2008 severe cholera-like disease, | 2 |
| | | | | | hospitalized patient | |
| 2127 | 1 | 2860650 | clinical ETEC isolate | icddr, b | CS5 + CS6 LT/ST 2008 severe cholera-like disease, | 2 |
| | | | | | hospitalized patient | |
| 2129 | 1 | 2862600 | clinical ETEC isolate | icddr, b | CS4 + CS6 ST 2008 severe cholera-like disease, hospitalized | 2 |
| | | | | | patient | |
| 2131 | 1 | 2864350 | clinical ETEC isolate | icddr, b | CF-ve LT/ST 2008 severe cholera-like disease, hospitalized | 2 |
| | | | | | patient | |
| 2132 | 1 | 2865200 | clinical ETEC isolate | icddr, b | CF-ve ST 2008 severe cholera-like disease, hospitalized | 2 |
| | | | | | patient | |
| 2133 | 1 | 2866350 | clinical ETEC isolate | icddr, b | CS5 + CS6 LT/ST 2008 severe cholera-like disease, | 2 |
| | | | | | hospitalized patient | |
| 2134 | 1 | 2866450 | clinical ETEC isolate | icddr, b | CS7 LT 2008 severe cholera-like disease, hospitalized | 2 |
| | | | | | patient | |
| 2135 | 1 | 2866550 | clinical ETEC isolate | icddr, b | CS7 LT 2008 severe cholera-like disease, hospitalized | 2 |
| | | | | | patient | |
| 2136 | 1 | 2866750 | clinical ETEC isolate | icddr, b | CS7 LT 2008 severe cholera-like disease, hospitalized | 2 |
| | | | | | patient | |
| 2137 | 1 | 2867750 | clinical ETEC isolate | icddr, b | CS7 LT 2008 severe cholera-like disease, hospitalized | 2 |
| | | | | | patient | |
| 2140 | 1 | 2872800 | clinical ETEC isolate | icddr, b | CS7 LT 2008 severe cholera-like disease, hospitalized | 2 |
| | | | | | patient | |
| 2142 | 1 | 2875150 | clinical ETEC isolate | icddr, b | CS4 + CS6 LT/ST 2008 severe cholera-like disease, | 2 |
| | | | | | hospitalized patient | |
| 2143 | 1 | 199900.1 | clinical ETEC isolate | icddr, b | CS17 LT severe cholera-like disease, hospitalized patient; | 2 |
| | | | | | isolate 1 | |
| 2155 | 1 | P0304816.1 | clinical ETEC isolate | icddr, b | STh, severe cholera-like disease, hospitalized patient, | 2 |
| | | | | | isolate 1 | |
| 2184 | 1 | p0305293.1 | clinical ETEC isolate | icddr, b | LT/STh severe cholera-like disease, hospitalized patient, | 2 |
| | | | | | isolate 1 | |
| 2216 | 1 | p0302308.1 | clinical ETEC isolate | icddr, b | LT/STh severe cholera-like disease, hospitalized patient, | 2 |
| | | | | | isolate 1 | |
| 2226 | 1 | p0304777.1 | clinical ETEC isolate | icddr, b | LT severe cholera-like disease, hospitalized patient, isolate 1 | 2 |
| 2267 | 1 | MP020940.1 | clinical ETEC isolate | icddr, b | STh, severe cholera-like disease, hospitalized patient, | 2 |
| | | | | | isolate 1 | |
| 2282 | 1 | P0298942.1 | clinical ETEC isolate | icddr, b | LT/STp, severe cholera-like disease, hospitalized patient, | 2 |
| | | | | | isolate 1 | |
| 2295 | 1 | p0299438.2 | clinical ETEC isolate | icddr, b | STh, severe cholera-like disease, hospitalized patient, | 2 |
| | | | | | isolate 2 | |
| 2305 | 1 | p0299917.1 | clinical ETEC isolate | icddr, b | LT/STh severe cholera-like disease, hospitalized patient, | 2 |
| | | | | | isolate 1 | |
| 2337 | 1 | 174900 | clinical ETEC isolate | icddr, b | CF-ve STh_2010 Diarr hospitalized patient | 2 |
| 2338 | 1 | 178200 | clinical ETEC isolate | icddr, b | CF-ve STh_2010 Diarr hospitalized patient | 2 |
| 2339 | 1 | 178850 | clinical ETEC isolate | icddr, b | CF-ve LT/STh_2010 Diarr hospitalized patient | 2 |
| 2342 | 1 | 179550 | clinical ETEC isolate | icddr, b | CS7 LT_2010 Diarr hospitalized patient | 2 |
| 2343 | 1 | 180200 | clinical ETEC isolate | icddr, b | CS7 LT/ST_2010 Diarr hospitalized patient | 2 |
| 2344 | 1 | 180600 | clinical ETEC isolate | icddr, b | CS17 LT/ST_2010 Diarr hospitalized patient | 2 |
| 2345 | 1 | 180050 | clinical ETEC isolate | icddr, b | CS17 LT_2010 Diarr hospitalized patient | 2 |
| 2063 | 1 | MP020980.1 | ETEC isolate | icddr, b | ST; patient with severe ETEC diarrhea at Mirpur colony 1 | 2 |
| 2356 | 1 | BCE001, | ETEC; | icddr, b | CF-ve LT_2002-04 Asymptomatic community | 0 |
| | | MS16 | asymptomatic | | | |
| 2357 | 1 | BCE002, | ETEC; | icddr, b | CS1 + CS3 LT/STh_2002-04 Asymptomatic community | 0 |
| | | MS12 | asymptomatic | | | |
| 2247 | 1 | p0301904.1 | non-ETEC. E. coli | icddr, b | non-ETEC hospitalized patient, isolate 1 | |
| | | | isolate | | | |
| 2255 | 1 | P0299483.1 | non-ETEC. E. coli | icddr, b | non-ETEC hospitalized patient, isolate 1 | |
| | | | isolate | | | |
| 2264 | 1 | P0304799.1 | non-ETEC. E. coli | icddr, b | non-ETEC hospitalized patient, isolate 1 | |
| | | | isolate | | | |
| 2149 | 0 | BCE006, MS- | asymptomatic | icddr, b | CS1 + CS3 LT + ST + 2004 asymptomatic colonization | 0 |
| | | 23 | colonization | | | |
| 2151 | 0 | BCE019, MS- | asymptomatic | icddr, b | CS1 + CS3 LT + ST + 2003 asymptomatic colonization | 0 |
| | | 13 | colonization | | | |
| 2152 | 0 | BCE030, MS- | asymptomatic | icddr, b | CFA/I ST + 2003 asymptomatic colonization | 0 |
| | | 09 | colonization | | | |
| 2153 | 0 | BCE032, MS- | asymptomatic | icddr, b | CS1 + CS3 LT + ST + 2003 asymptomatic colonization | 0 |
| | | 12 | colonization | | | |
| 350 | 0 | ETEC | clinical ETEC isolate | R. A. Finkelstein | serotype 063:Nm | 2 |
| | | ThroopD “1” | | | | |
| 1390 | 0 | ETEC Jurua | clinical ETEC isolate | from Brazil | CS7; LT; severe cholera-like disease from 1998 | 2 |
| | | 18/11 | | | | |
| 1391 | 0 | ETEC Jurua | clinical ETEC isolate | from Brazil | CS7; LT; severe cholera-like disease from 1998 | 2 |
| | | 20/10 | | | | |
| 2098 | 0 | 2719100 | clinical ETEC isolate | icddr, b | CFA/I + CS21; ST; mild, self limited diarrhea from 2007 | 1 |
| 2104 | 0 | 2741950 | clinical ETEC isolate | icddr, b | CS1 + CS3 + CS21 LT/ST 2007 severe cholera-like disease, | 2 |
| | | | | | hospitalized patient | |
| 2107 | 0 | 2730450 | clinical ETEC isolate | icddr, b | CS1 + CS3 + CS21 LT/ST 2007 severe cholera-like disease, | 2 |
| | | | | | hospitalized patient | |
| 2119 | 0 | 2846750 | clinical ETEC isolate | icddr, b | CS2 + CS3 LT/ST 2008 mild, self-limited diarrhea | 1 |
| 2123 | 0 | 2851500 | clinical ETEC isolate | icddr, b | CS7 LT 2008 mild, self-limited diarrhea | 1 |
| 2128 | 0 | 2861200 | clinical ETEC isolate | icddr, b | CS6 ST 2008 severe cholera-like disease, hospitalized | 2 |
| | | | | | patient | |
| 2130 | 0 | 2863500 | clinical ETEC isolate | icddr, b | CFA/I ST 2008 severe cholera-like disease, hospitalized | 2 |
| | | | | | patient | |
| 2138 | 0 | 2871950 | clinical ETEC isolate | icddr, b | CS2 + CS3 LT 2008 severe cholera-like disease, hospitalized | 2 |
| | | | | | patient | |
| 2139 | 0 | 2872000 | clinical ETEC isolate | icddr, b | CS2 + CS3 LT 2008 severe cholera-like disease, hospitalized | 2 |
| | | | | | patient | |
| 2141 | 0 | 2875000 | clinical ETEC isolate | icddr, b | CS7 LT 2008 severe cholera-like disease, hospitalized | 2 |
| | | | | | patient | |
| 2145 | 0 | 21600.1 | clinical ETEC isolate | icddr, b | CS14 LT/ST severe cholera-like disease, hospitalized | 2 |
| | | | | | patient; isolate 1 | |
| 2199 | 0 | p0301867.1 | clinical ETEC isolate | icddr, b | STh, severe cholera-like disease, hospitalized patient, | 2 |
| | | | | | isolate 1 | |
| 2208 | 0 | p0302293.2 | clinical ETEC isolate | icddr, b | LT/STh severe cholera-like disease, hospitalized patient, | 2 |
| | | | | | isolate 2 | |
| 2336 | 0 | 174750 | clinical ETEC isolate | icddr, b | CS1 + CS3 + CS21 LT/STh_2010 Diarr hospitalized patient | 2 |
| 2340 | 0 | 178900 | clinical ETEC isolate | icddr, b | CFA/I, CS21 STh_2010 Diarr hospitalized patient | 2 |
| 2341 | 0 | 179100 | clinical ETEC isolate | icddr, b | CF-ve LT/STh_2010 Diarr hospitalized patient | 2 |
| 2346 | 0 | C-34666 | clinical ETEC isolate | icddr, b | CF-ve LT/STh_2010 Diarr hospitalized patient | 2 |
| 2381 | 0 | mp021566.1 | clinical ETEC isolate | icddr, b | LT/STh, Mirpur patient, isolate 1 | 2 |
| 2007 | 0 | P02997067.1 | ETEC isolate | icddr, b | LT/STh; patient with severe ETEC diarrhea at icddr, b | 2 |
| | | | | | colony 1 | |
| 2021 | 0 | MP021552.1 | ETEC isolate | icddr, b | LT/STh; patient with severe ETEC diarrhea at Mirpur colony 1 | 2 |
| 2036 | 0 | MP021561.1 | ETEC isolate | icddr, b | STh; patient with severe ETEC diarrhea at Mirpur colony 1 | 2 |
| 2053 | 0 | MP021017.1 | ETEC isolate | icddr, b | ST; patient with severe ETEC diarrhea at Mirpur colony 1 | 2 |
| 2359 | 0 | BCE007, | ETEC; | icddr, b | CS1 + CS3 + CS21 LT/STh_2002-04 Asymptomatic | 0 |
| | | MS11 | asymptomatic | | community | |
| 2360 | 0 | BCE008, | ETEC; | icddr, b | CF-ve LT_2002-04 Asymptomatic community | 0 |
| | | MS1 | asymptomatic | | | |
| 2361 | 0 | BCE008, | ETEC; | icddr, b | CS6 STp_2002-04 Asymptomatic community | 0 |
| | | MS13 | asymptomatic | | | |
| 2358 | | BCE005, | ETEC; | icddr, b | CF-ve LT/STh_2002-04 Asymptomatic community | 0 |
| | | MS23 | asymptomatic | | | |
| | 63 | | | | | |

60/91 GSCID ETEC isolates have EaeH gene in the genome.

### REFERENCES FOR THE EXAMPLES

1. Kotloff K L, Nataro J P, Blackwelder W C, Nasrin D, Farag T H, et al. (2013) Burden and aetiology of diar-rhoeal disease in infants and young children in developing countries (the Global Enteric Multicenter Study, GEMS): a prospective, case-control study. Lancet doi: 10.1016/S0140-6736(13)60844-2 PMID: 23680352
2. Fleckenstein J M, Hardwidge P R, Munson G P, Rasko D A, Sommerfelt H, et al. (2010) Molecular mech [symbol] anisms of enterotoxigenic Escherichia coli infection. Microbes Infect 12: 89-98. doi: 10.1016/j.micinf. 2009.10.002 PMID: 19883790
3. Evans D G, Silver R P, Evans D J Jr., Chase D G, Gorbach S L (1975) Plasmid-controlled colonization fa [symbol] ctor associated with virulence in Escherichia coli enterotoxigenic for humans. Infect Immun 12: 656-667. PMID: 1100526
4. Carpenter C C, Barua D, Wallace C K, Sack R B, Mitra P P, et al. (1965) Clinical and physiological obser [symbol] vations during an epidemic outbreak of non-vibrio cholera-like disease in Calcutta. Bull World Health Organ 33: 665-671. PMID: 5295147
5. Gorbach S L, Banwell J G, Chatterjee B D, Jacobs B, Sack R B (1971) Acute undifferentiated human diar [symbol] rhea in the tropics. I. Alterations in intestinal micrflora. J Clin Invest 50: 881-889. doi: 10.1172/JCI106560 PMID: 4926260
6. Sack R B, Gorbach S L, Banwell J G, Jacobs B, Chatterjee B D, et al. (1971) Enterotoxigenic Escherichia coli isolated from patients with severe cholera-like disease. J Infect Dis 123: 378-385. doi: 10.1093/infdis/123.4.378 PMID: 4938945
7. Evans D G, Satterwhite T K, Evans D J Jr., DuPont H L (1978) Differences in serological responses and excretion patterns of volunteers challenged with enterotoxigenic Escherichia coli with and without the colonization factor antigen. Infect Immun 19: 883-888. PMID: 346488
8. Satterwhite T K, Evans D G, DuPont H L, Evans D J Jr. (1978) Role of Escherichia coli colonisation factor antigen in acute diarrhoea. Lancet 2: 181-184. doi: 10.1016/S0140-6736(78)91921-9 PMID: 78384
9. Svennerholm A M, Lundgren A (2012) Recent progress toward an enterotoxigenic Escherichia coli vac [symbol] cine. Expert review of vaccines 11: 495-507. doi: 10.1586/erv.12.12 PMID: 22551034
10. Zhang W, Sack D A (2012) Progress and hurdles in the development of vaccines against enterotoxigen-ic Escherichia coli in humans. Expert review of vaccines 11: 677-694. doi: 10.1586/erv.12.37 PMID: 22873126
11. Rasko D A, Rosovitz M J, Myers G S, Mongodin E F, Fricke W F, et al. (2008) The pangenome structure of Escherichia coli: comparative genomic analysis of E. coli commensal and pathogenic isolates. J Bac-teriol 190: 6881-6893. doi: 10.1128/JB.00619-08 PMID: 18676672
12. Del Canto F, Botkin D J, Valenzuela P, Popov V, Ruiz-Perez F, et al. (2012) Identification of the Coli Sur [symbol] face Antigen 23 (CS23), a Novel Adhesin of Enterotoxigenic Escherichia coli. Infection and immunity doi: 10.1128/IAI.00263-12 PMID: 22645287
13. Isidean S D, Riddle M S, Savarino S J, Porter C K (2011) A systematic review of ETEC epidemiology fo [symbol] cusing on colonization factor and toxin expression. Vaccine 29: 6167-6178. doi: 10.1016/j.vaccine. 2011.06.084 PMID: 21723899
14. Riddle M S, Savarino S J (2013) Moving beyond a heat-labile enterotoxin-based vaccine against entero-toxigenic Escherichia coli. Lancet Infect Dis. doi: 10.1016/S1473-3099(13)70355-4 PMID: 24291167
15. Behrens R H, Cramer J P, Jelinek T, Shaw H, von Sonnenburg F, et al. (2013) Efficacy and safety of a patch vaccine containing heat-labile toxin from Escherichia coli against travellers' diarrhoea: a phase 3, randomised, double-blind, placebo-controlled field trial in travellers from Europe to Mexico and Guate [symbol] mala. Lancet Infect Dis. doi: 10.1016/S1473-3099(13)70297-4 PMID: 24291168
16. Darsley M J, Chakraborty S, Denearing B, Sack D A, Feller A, et al. (2012) ACE527 Oral, Live Attenuat [symbol] ed ETEC Vaccine Reduces the Incidence and Severity of Diarrhea in a Human Challenge Model of Di-arrheal Disease. Clin Vaccine Immunol doi: 10.1128/CVI.00364-12 PMID: 23035175
17. Harro C, Sack D, Bourgeois A L, Walker R, Denearing B, et al. (2011) A combination vaccine consisting of three live attenuated enterotoxigenic Escherichia coli strains expressing a range of colonization fac [symbol] tors and LTB is well tolerated and immunogenic in a placebo-controlled double-blind Phase I trial in healthy adults. Clin Vaccine Immunol doi: 10.1128/CVI.05342-11 PMID: 21994354
18. Turner A K, Stephens J C, Beavis J C, Greenwood J, Gewert C, et al. (2011) Generation and characteri [symbol] zation of a live attenuated enterotoxigenic Escherichia coli combination vaccine expressing six coloni [symbol] zation factors and heat-labile toxin subunit B. Clin Vaccine Immunol 18: 2128-2135. doi: 10.1128/CVI. 05345-11 PMID: 21994355
19. Fleckenstein J M, Roy K, Fischer J F, Burkitt M (2006) Identification of a two-partner secretion locus of enterotoxigenic Escherichia coli. Infect Immun 74: 2245-2258. doi: 10.1128/IAI.74.4.2245-2258.2006 PMID: 16552055
20. Roy K, Hilliard G M, Hamilton D J, Luo J, Ostmann M M, et al. (2009) Enterotoxigenic Escherichia coli EtpA mediates adhesion between flagella and host cells. Nature 457: 594-598. doi: 10.1038/nature07568 PMID: 19060885
21. Patel S K, Dotson J, Allen K P, Fleckenstein J M (2004) Identification and molecular characterization of EatA, an autotransporter protein of enterotoxigenic Escherichia coli. Infect Immun 72: 1786-1794. doi: 10.1128/IAI.72.3.1786-1794.2004 PMID: 14977988
22. Roy K, Kansal R, Bartels S R, Hamilton D J, Shaaban S, et al. (2011) Adhesin Degradation Accelerates Delivery of Heat-labile Toxin by Enterotoxigenic Escherichia coli. J Biol Chem 286: 29771-29779. doi: 10.1074/jbc.M111.251546 PMID: 21757737
23. Kumar P, Luo Q, Vickers T J, Sheikh A, Lewis W G, et al. (2013) EatA, an Immununogenic Protective Antigen of Enterotoxigenic Escherichia coli Degrades Intestinal Mucin. Infect Immun. doi: 10.1128/IAI. 01078-13 PMID: 24478066
24. Johansson M E, Sjovall H, Hansson G C (2013) The gastrointestinal mucus system in health and dis [symbol] ease. Nat Rev Gastroenterol Hepatol 10: 352-361. doi: 10.1038/nrgastro.2013.35 PMID: 23478383
25. Roy K, Bartels S, Qadri F, Fleckenstein J M (2010) Enterotoxigenic Escherichia coli elicits immune re-sponses to multiple surface proteins. Infect Immun 78: 3027-3035. doi: 10.1128/IAI.00264-10 PMID: 20457787
26. Kansal R, Rasko D A, Sahl J W, Munson G P, Roy K, et al. (2013) Transcriptional modulation of entero-toxigenic Escherichia coli virulence genes in response to epithelial cell interactions. Infect Immun 81: 259-270. doi: 10.1128/IAI.00919-12 PMID: 23115039
27. Luo Q, Kumar P, Vickers T J, Sheikh A, Lewis W G, et al. (2014) Enterotoxigenic Escherichia coli Se [symbol] cretes a Highly Conserved Mucin-Degrading Metalloprotease To Effectively Engage Intestinal Epitheli [symbol] al Cells. Infect Immun 82: 509-521. doi: 10.1128/IAI.01106-13 PMID: 24478067
28. Sheikh A, Lou Q, Roy K, Shabaan S, Kumar P, et al. (2014) Contribution of the highly conserved EaeH surface protein to enterotoxigenic Escherichia coli pathogenesis. Infect Immun doi: 10.1128/IAI.01890-14 PMID: 24935979
29. Kumar P, Luo Q, Vickers T J, Sheikh A, Lewis W G, et al. (2014) EatA, an Immunogenic Protective Anti [symbol] gen of Enterotoxigenic Escherichia coli, Degrades Intestinal Mucin. Infect Immun 82: 500-508. doi: 10. 1128/IAI.01078-13 PMID: 24478066
30. Roy K, Hamilton D J, Fleckenstein J M (2012) Cooperative role of antibodies against heat-labile toxin and the EtpA Adhesin in preventing toxin delivery and intestinal colonization by enterotoxigenic Escher-ichia coli. Clin Vaccine Immunol 19: 1603-1608. doi: 10.1128/CVI.00351-12 PMID: 22875600
31. Roy K, Hamilton D, Ostmann M M, Fleckenstein J M (2009) Vaccination with EtpA glycoprotein or flagel-lin protects against colonization with enterotoxigenic Escherichia coli in a murine model. Vaccine 27: 4601-4608. doi: 10.1016/j.vaccine.2009.05.076 PMID: 19523914
32. Mundell D H, Anselmo C R, Wishnow R M (1976) Factors influencing heat-labile Escherichia coli entero-toxin activity. Infect Immun 14: 383-388. PMID: 9363
33. Alam N H, Ashraf H (2003) Treatment of infectious diarrhea in children. Paediatric drugs 5: 151-165. PMID: 12608880
34. Sjoling A, Wiklund G, Savarino S J, Cohen D I, Svennerholm A M (2007) Comparative analyses of phe [symbol] notypic and genotypic methods for detection of enterotoxigenic Escherichia coli toxins and colonization factors. J Clin Microbiol 45: 3295-3301. doi: 10.1128/JCM.00471-07 PMID: 17687011
35. Vicente A C, Teixeira L F, Iniguez-Rojas L, Luna M G, Silva L, et al. (2005) Outbreaks of cholera-like diar [symbol] rhoea caused by enterotoxigenic Escherichia coli in the Brazilian Amazon Rainforest. Trans R Soc Trop Med Hyg 99: 669-674. doi: 10.1016/j.trstmh.2005.03.007 PMID: 15975612
36. Finkelstein R A, Vasil M L, Jones J R, Anderson R A, Barnard T (1976) Clinical cholera caused by entero-toxigenic Escherichia coli. J Clin Microbiol 3: 382-384. PMID: 773963
37. Del Canto F, Valenzuela P, Cantero L, Bronstein J, Blanco J E, et al. (2011) Distribution of Classical and Nonclassical Virulence Genes in Enterotoxigenic Escherichia coli Isolates from Chilean Children and tRNA Gene Screening for Putative Insertion Sites for Genomic Islands. J Clin Microbiol 49: 3198-3203. doi: 10.1128/JCM.02473-10 PMID: 21775541
38. Harlow E, Lane D, Harlow E (1999) Using antibodies: a laboratory manual. Cold Spring Harbor, N.Y.: Cold Spring Harbor Laboratory Press. xiv, 495 p. p.
39. Altschul S F, Gish W, Miller W, Myers E W, Lipman D J (1990) Basic local alignment search tool. J Mol Biol 215: 403-410. doi: 10.1006/jmbi.1990.9999 PMID: 2231712
40. Sievers F, Wilm A, Dineen D, Gibson T J, Karplus K, et al. (2011) Fast, scalable generation of high-qual [symbol] ity protein multiple sequence alignments using Clustal Omega. Molecular systems biology 7: 539. doi: 10.1038/msb.2011.75 PMID: 21988835
41. Team R C (2014) R: A language and environment for statistical computing. Vienna, Austria: R Founda [symbol] tion for Statistical Computing.
42. Warnes G, Bolker B, Bonebakker L, Gentleman R, Huber W, et al. (2014) gplots: Various R program [symbol] ming tools for plotting data. R package version 2.13.0. ed.
43. Neuwirth E (2011) RColorBrewer: ColorBrewer palettes. R package version 1.0-5 ed.
44. Sheikh A, Luo Q, Roy K, Shaaban S, Kumar P, et al. Contribution of the highly conserved EaeH surface protein to enterotoxigenic Escherichia coli pathogenesis. Infect Immun in press.
45. Fleckenstein J M, Roy K (2009) Purification of recombinant high molecular weight two-partner secretion proteins from Escherichia coli. Nat Protoc 4: 1083-1092. doi: 10.1038/nprot.2009.87 PMID: 19707189
46. Tsang V C, Wilson B C, Maddison S E (1980) Kinetic studies of a quantitative single-tube enzyme-linked immunosorbent assay. Clinical chemistry 26: 1255-1260. PMID: 6772340
47. Harris J A, Roy K, Woo-Rasberry V, Hamilton D J, Kansal R, et al. (2011) Directed evaluation of entero-toxigenic Escherichia coli autotransporter proteins as putative vaccine candidates. PLoS Negl Trop Dis 5: e1428. doi: 10.1371/journal.pntd.0001428 PMID: 22163060
48. Dorsey F C, Fischer J F, Fleckenstein J M (2006) Directed delivery of heat-labile enterotoxin by entero-toxigenic Escherichia coli. Cell Microbiol 8: 1516-1527. doi: 10.1111/j.1462-5822.2006.00736.x PMID: 16922869
49. Sahl J W, Steinsland H, Redman J C, Angiuoli S V, Nataro J P, et al. (2011) A comparative genomic anal [symbol] ysis of diverse clonal types of enterotoxigenic Escherichia coli reveals pathovar-specific conservation. Infect Immun 79: 950-960. doi: 10.1128/IAI.00932-10 PMID: 21078854
50. Svennerholm A M, Vidal Y L, Holmgren J, McConnell M M, Rowe B (1988) Role of PCF8775 antigen and its coli surface subcomponents for colonization, disease, and protective immunogenicity of enterotoxi-genic Escherichia coli in rabbits. Infection and immunity 56: 523-528. PMID: 3276628
51. McConnell M M, Thomas L V, Willshaw G A, Smith H R, Rowe B (1988) Genetic control and properties of coli surface antigens of colonization factor antigen IV (PCF8775) of enterotoxigenic Escherichia coli. In [symbol] fect Immun 56: 1974-1980. PMID: 2456269
52. Evans D J Jr., Evans D G (1973) Three characteristics associated with enterotoxigenic Escherichia coli isolated from man. Infect Immun 8: 322-328. PMID: 4581006
53. Coster T S, Wolf M K, Hall E R, Cassels F J, Taylor D N, et al. (2007) Immune response, ciprofloxacin ac [symbol] tivity, and gender differences after human experimental challenge by two strains of enterotoxigenic Escherichia coli. Infect Immun 75: 252-259. doi: 10.1128/IAI.01131-06 PMID: 17074855
54. Chen Q, Savarino S J, Venkatesan M M (2006) Subtractive hybridization and optical mapping of the en-terotoxigenic Escherichia coli H10407 chromosome: isolation of unique sequences and demonstration of significant similarity to the chromosome of E. coli K-12. Microbiology 152: 1041-1054. doi: 10.1099/mic.0.28648-0 PMID: 16549668
55. Moriel D G, Bertoldi I, Spagnuolo A, Marchi S, Rosini R, et al. (2010) Identification of protective and broadly conserved vaccine antigens from the genome of extraintestinal pathogenic Escherichia coli. Proc Natl Acad Sci USA 107: 9072-9077. doi: 10.1073/pnas.0915077107 PMID: 20439758
56. Roy K, Hamilton D, Allen K P, Randolph M P, Fleckenstein J M (2008) The EtpA exoprotein of enterotoxi-genic Escherichia coli promotes intestinal colonization and is a protective antigen in an experimental model of murine infection. Infect Immun 76: 2106-2112. doi: 10.1128/IAI.01304-07 PMID: 18285493
57. Sack R B (2011) The discovery of cholera—like enterotoxins produced by Escherichia coli causing se [symbol] cretory diarrhoea in humans. The Indian journal of medical research 133: 171-180. PMID: 21415491
58. Sheikh A, Luo Q, Roy K, Shabaan S, Kumar P, et al. (2014) Contribution of the highly conserved EaeH surface protein to enterotoxigenic Escherichia coli pathogenesis. Infect Immun 82: 3657-3666. doi: 10. 1128/IAI.01890-14 PMID: 24935979
59. Fleckenstein J M, Sheikh A (2014) Designing vaccines to neutralize effective toxin delivery by entero-toxigenic Escherichia coli. Toxins (Basel) 6: 1799-1812. doi: 10.3390/toxins6061799 PMID: 24918359
60. Fleckenstein J, Sheikh A, Qadri F (2014) Novel antigens for enterotoxigenic Escherichia coli vaccines. Expert review of vaccines 13: 631-639. doi: 10.1586/14760584.2014.905745 PMID: 24702311
61. Crossman L C, Chaudhuri R R, Beatson S A, Wells T J, Desvaux M, et al. (2010) A commensal gone bad: complete genome sequence of the prototypical enterotoxigenic Escherichia coli strain H10407. Journal of bacteriology 192: 5822-5831. doi: 10.1128/JB.00710-10 PMID: 20802035
62. Madhavan T P, Steen J A, Hugenholtz P, Sakellaris H (2014) Genome Sequence of Enterotoxigenic Escherichia coli Strain B2C. Genome announcements 2. doi: 10.1128/genomeA.00247-14 PMID: 24723709
63. Scotland S M, McConnell M M, Willshaw G A, Rowe B, Field A M (1985) Properties of wild-type strains of enterotoxigenic Escherichia coli which produce colonization factor antigen II, and belong to serogroups other than 06. J Gen Microbiol 131: 2327-2333. PMID: 3906040

1. A vaccine composition, the vaccine composition comprising EtpA and EatA.

2. The vaccine composition of claim 1, wherein the EatA comprises a mutation that disrupts serine protease activity.

3. The vaccine composition of claim 2, wherein the mutation is one or more mutations at histidine 134, aspartic acid 162, serine 267.

4. The vaccine composition of claim 3, wherein the mutation is one or more mutations selected from the group consisting of H134A, D162A, and S267G.

5. The vaccine composition of claim 1, wherein the EatA comprises the passenger domain.

6. The vaccine composition of claim 1, wherein the EatA comprises about 80% identity to SEQ ID NO: 1 or SEQ ID NO:2.

7. The vaccine composition of claim 1, wherein the EatA comprises at least 95% identity to SEQ ID NO: 1 or SEQ ID NO:2.

8. The vaccine composition of claim 1, wherein the EatA comprises SEQ ID NO: 1 or SEQ ID NO:2.

9. The vaccine composition of claim 3, wherein the EatA comprises about 80% identity to SEQ ID NO: 1 or SEQ ID NO:2.

10. The vaccine composition of claim 3, wherein the EatA comprises at least 95% identity to SEQ ID NO: 1 or SEQ ID NO:2.

11. The vaccine composition of claim 1, wherein the EtpA comprises the secreted EtpA portion.

12. The vaccine composition of claim 1, wherein the EtpA comprises about 80% identity to SEQ ID NO:3 or SEQ ID NO:4.

13. (canceled)

14. The vaccine composition of claim 1, wherein the EtpA comprises SEQ ID NO:3 or SEQ ID NO:4.

15. The vaccine composition of claim 1, wherein the vaccine composition comprises EtpA and EatA linked together.

16. The vaccine composition of claim 1, further comprising colonization factor antigens.

17. The vaccine composition of claim 1, wherein the vaccine composition comprises a suitable adjuvant.

18. The vaccine composition of claim 17, wherein the adjuvant is selected from the group consisting of heat-labile toxin and double mutant heat-labile toxin (dmLT).

19. A method of protecting against intestinal colonization of enterotoxigenic Escherichia coli (ETEC) in a subject, the method comprising administering to the subject an effective amount of a vaccine composition comprising EatA and EtpA.

20. A method of preventing or treating ETEC-associated diarrhea, the method comprising administering to the subject an effective amount of a vaccine composition comprising EatA and EtpA.

21. (canceled)

22. The method of claim 19, wherein the vaccine composition is administered at a dose ranging from about 15 to about 100 g.

23.-26. (canceled)


SEQUENCE LISTING (32 sequences)

SEQ ID NO: 1 - protein, 1364 aa, Escherichia coli
MNKVFSLKYSFLAKGFIAVSELARRVSVKGKLKSASSIIISPITIAIVSYAPPSLAATVNADISYQTFRDFAENKGAFIVGASNINIYDKNGVLVGVLDKAPMPDFSSATMNTGTLPPGDHTLYSPQYVVTAKHVNGSDIMSFGHIQNNYTVVGENNHNSLDIKIRRLNKIVTEVAPAEISSVGAVNGAYQEGGRFKAFYRLGGGLQYIKDKNGNLTPVYTNGGFLTGGTISALSSYNNGQMITAPTGDIFNPANGPLANYLNKGDSGSPLFAYDSLDKKWVLVGVLSSGSEHGNNWVVTTQDFLHQQPKHDFDKTISYDSEKGSLQWRYNKNSGVGTLSQESVVWDMHGKKGGDLNAGKNLQFTGNNGEIILHDSIDQGAGYLQFFDNYTVTSLTDQTWTGGGIITEKGVNVLWQVNGVNDDNLHKVGEGTLTVNGKGVNNGGLKVGDGTVILNQRPDDNGHKQAFSSINISSGRATVILSDANQVNPDKISWGYRGGTLDLNGNNVNFTRLQAADYGAIVSNNNKNKSELTLKLQTLNENDISVDVKTYEVFGGHGSPGDLYYVPASNTYFILKSKAYGPFFSDLDNTNVWQNVGHDRDKAIQIVKQQKIGESSQPYMFHGQLNGYMDVNIHPLSGKDVLTLDGSVNLPEGVITKKSGTLIFQGHPVIHAGMTTSAGQSDWENRQFTMDKLRLDAATFHLSRNAHMQGDISAANGSTVILGSSRVFTDKNDGTGNAVSSVEGSSIATTAGDQSYYSGNVLLENHSSLEVRENFTGGIEAYDSSVSVTSQNAIFDHVGSFVNSSLLLEKGAKLTAQSGIFTNNTMKIKENASLTLTGIPSVGKPGYYSPVTSTTEGIHLGERASLSVKNMGYLSSNITAENSAAIINLGDSNATIGKTDSPLFSTLMRGYNAVLQGNIMGPQSSVNMNNALWHSDRNSELKELKANDSQIELGVRGHFAKLRVKELIASNSVFLVHANNSQADQLNVTDKLQGSNNTILVDFFNKAANGTNVTLITAPKGSDENTFKAGTQQIGFSNITPEIRTENTDTATQWVLTGYQSVADARASKIATDFMDSGYKSFLTEVNNLNKRMGDLRDSQGDAGGWARIMNGTGSGESGYRDNYTHVQIGADRKHELNGIDLFTGALLTYTDNNASSQAFSGKTKSLGGGVYASGLFESGAYFDLIGKYLHHDNRYTLNFASLGERSYTSHSLYAGAEIGYRYHMSENTWVEPQMELVYGSVSGKSFNWKDQGMQLSMKDKDYHPLIGRTGVDVGRAFSGDTWKVTVRAGLGYQFDLLANGETVLQDASGKKHFKGEKDSRMLMNVGTNVEVKDNMRFGLELEKSAFGRYNIDNSINANFRYYF

SEQ ID NO: 2 - protein, 1042 aa, Escherichia coli
ATVNADISYQTFRDFAENKGAFIVGASNINIYDKNGVLVGVLDKAPMPDFSSATMNTGTLPPGDHTLYSPQYVVTAKHVNGSDIMSFGHIQNNYTVVGENNHNSLDIKIRRLNKIVTEVAPAEISSVGAVNGAYQEGGRFKAFYRLGGGLQYIKDKNGNLTPVYTNGGFLTGGTISALSSYNNGQMITAPTGDIFNPANGPLANYLNKGDSGSPLFAYDSLDKKWVLVGVLSSGSEHGNNWVVTTQDFLHQQPKHDFDKTISYDSEKGSLQWRYNKNSGVGTLSQESVVWDMHGKKGGDLNAGKNLQFTGNNGEIILHDSIDQGAGYLQFFDNYTVTSLTDQTWTGGGIITEKGVNVLWQVNGVNDDNLHKVGEGTLTVNGKGVNNGGLKVGDGTVILNQRPDDNGHKQAFSSINISSGRATVILSDANQVNPDKISWGYRGGTLDLNGNNVNFTRLQAADYGAIVSNNNKNKSELTLKLQTLNENDISVDVKTYEVFGGHGSPGDLYYVPASNTYFILKSKAYGPFFSDLDNTNVWQNVGHDRDKAIQIVKQQKIGESSQPYMFHGQLNGYMDVNIHPLSGKDVLTLDGSVNLPEGVITKKSGTLIFQGHPVIHAGMTTSAGQSDWENRQFTMDKLRLDAATFHLSRNAHMQGDISAANGSTVILGSSRVFTDKNDGTGNAVSSVEGSSIATTAGDQSYYSGNVLLENHSSLEVRENFTGGIEAYDSSVSVTSQNAIFDHVGSFVNSSLLLEKGAKLTAQSGIFTNNTMKIKENASLTLTGIPSVGKPGYYSPVTSTTEGIHLGERASLSVKNMGYLSSNITAENSAAIINLGDSNATIGKTDSPLFSTLMRGYNAVLQGNIMGPQSSVNMNNALWHSDRNSELKELKANDSQIELGVRGHFAKLRVKELIASNSVFLVHANNSQADQLNVTDKLQGSNNTILVDFFNKAANGTNVTLITAPKGSDENTFKAGTQQIGFSNITPEIRTENTDTATQWVLTGYQSVADARASKIATDFMDSGYKSFLTEVNNLNKRMGDLRD

SEQ ID NO: 3 - protein, 1767 aa, Escherichia coli
MNRIYKLKFDKRRNELVVVSEITTGVGNAKATGSVEGEKSPRRGVRAMALSLLSGMMIMAHPAMSANLPTGGQIVAGSGSIQTPSGNQMNIHQNSQNMVANWNSFDIGKGNTVQFDQPSSSAVALNRVVGGGESQIMGNLKANGQVFLVNPNGVLFGEGASVSTSGFVASTRDIKNDDFMNRRYTFSGGQKAGAAIVNQGELTTNAGGYIVLAADRVSNSGTIRTPGGKTVLAASERITLQLDNGGLMSVQVTGDVVNALVENRGLVSARDGQVYLTALGRGMLMNTVLNVSGVVEASGMHRQDGNIVLDGGDSGVVHLSGTLQADNASGQGGKVVVQGKNILLDKGSNITATGGQGGGEVYVGGGWQGKDSNIRNADKVVMQGGARIDVSATQQGNGGTAVLWSDSYTNFHGQISAKGGETGGNGGRVETSSHGNLQAFGTVSASAKKGKAGNWLLDSADITIVNGSNVSKTETTQSPPHTQFAPTAAGSAVSNTSINNRLNNGTSVTILTHRTRTGTAQGGNITVNAAINKSNGSDVNLTLQAGGNITVNNSITSTEGKLNVNLSGARTSNGSITISNNANITTNGGDITVGTTNTSNRVNISINNTTLNASNGNIQLTGTGTDSGILFAGNNRLTASNIALTGNSTSGNAINLTGTATLNATNNITLTGSSTSGNAINLKGNNTLTASNITLTGESTSGNAINLTDTTGTTTLNATNNITMQGTRVQIKHSNITAGNFALNATVAGSEISNTTLTATNNINLAAKTNSASSGVYLKDARITSTNGSITANGTATANGKATHLDGNVTLNASNGRIKLTGNGHGSASGILFAGNNRLTASNIALTGNSTSGNAINLTGTATLNATNDITLTGSSTSGNAINLTGTATLNATNNITLTGSSTSGNAINLKGNNTLTASNITLTGESTSGNAINLTDTTGTTTLNATNNITMQGTRVQIKHSNITAGNFALNATVAGSEISNTTLTATNNINLAAKTNSASSGVYLKDARITSTNGSITANGTATANGKATHLDGNVTLNASNGRIKLTGNGHGSASGILFAGNNRLTASNIALTGNSTSGNAINLTGTATLNATNDITLTGSSTSGNAINLTGTATLNATNNITLTGSSTSGNAINLKGNNTLTASNITLTGESTSGNAINLTDTTGTTTLNATNNITMQGTRVQIKHSNITAGNFALNATVAGSEISNTTLTATNNINLAAKTNSASSGVYLKDARITSTNGSITANGTATANGKATHLDGNVTLNASNGRIKLTGNGHGSASGILFAGNNRLTASNIALTGNSTSGNAINLTGTATLNATNDITLTGSSTSGNAINLTGTATLNATNNITLTGSSTSGNAINLKGNNTLTASNITLTGESTSGNAINLTDTTGTTTLNATNNITMQGTRVQIKHSNITAGNFALNATVAGSEISNTTLTATNNINLAAKTNSASSGVYLKDARITSTNGSITTNGTATANGKATHLDGNVTLNASNGRIKLTGNGHGSASGILFAGNNRLTASNIALTGNSTSGNAINLTGTATLNATNDITLTGSSTSGNAINLTGTATLNATNNITLTGSSTSGNAINLKGNNTLTASNITLTGESTSGNAINLTDTTGTTTLNATNNITMQGTRVQIKHSNITAGNFALNATVAGSEISNTTLTATNNINLAAKTNSASSGVYLKDARITSTNGSITANGTAPANDNATYLDGNVTLNASNGSIKLTGNGNGSTSGILFAGNNTLTASNITLTGNSEVYWQ

SEQ ID NO: 4 - protein, 600 aa, Escherichia coli
MNRIYKLKFDKRRNELVVVSEITTGVGNAKATGSVEGEKSPRRGVRAMALSLLSGMMIMAHPAMSANLPTGGQIVAGSGSIQTPSGNQMNIHQNSQNMVANWNSFDIGKGNTVQFDQPSSSAVALNRVVGGGESQIMGNLKANGQVFLVNPNGVLFGEGASVSTSGFVASTRDIKNDDFMNRRYTFSGGQKAGAAIVNQGELTTNAGGYIVLAADRVSNSGTIRTPGGKTVLAASERITLQLDNGGLMSVQVTGDVVNALVENRGLVSARDGQVYLTALGRGMLMNTVLNVSGVVEASGMHRQDGNIVLDGGDSGVVHLSGTLQADNASGQGGKVVVQGKNILLDKGSNITATGGQGGGEVYVGGGWQGKDSNIRNADKVVMQGGARIDVSATQQGNGGTAVLWSDSYTNFHGQISAKGGETGGNGGRVETSSHGNLQAFGTVSASAKKGKAGNWLLDSADITIVNGSNVSKTETTQSPPHTQFAPTAAGSAVSNTSINNRLNNGTSVTILTHRTRTGTAQGGNITVNAAINKSNGSDVNLTLQAGGNITVNNSITSTEGKLNVNLSGARTSNGSITISNNANITTNGGDITVGTTNTSN

SEQ ID NO: 5 - protein, 5 aa, Escherichia coli
NPNGV

SEQ ID NO: 6 - DNA, 20 bp, Artificial Sequence
  SYNTHESIZED
atgtgctttggcaggttaat

SEQ ID NO: 7 - DNA, 20 bp, Artificial Sequence
  SYNTHESIZED
atatccagtcagcacccact

SEQ ID NO: 8 - DNA, 20 bp, Artificial Sequence
  SYNTHESIZED
ggttcaggcagtatccagac

SEQ ID NO: 9 - DNA, 20 bp, Artificial Sequence
  SYNTHESIZED
ggtgtagctgtctgaccaca

SEQ ID NO: 10 - DNA, 19 bp, Artificial Sequence
  SYNTHESIZED
acggcgttactatcctctc

SEQ ID NO: 11 - DNA, 20 bp, Artificial Sequence
  SYNTHESIZED
tggtctcggtcagatatgtg

SEQ ID NO: 12 - DNA, 21 bp, Artificial Sequence
  SYNTHESIZED
tctttcccctcttttagtcag

SEQ ID NO: 13 - DNA, 21 bp, Artificial Sequence
  SYNTHESIZED
acaggcaggattacaacaaag

SEQ ID NO: 14 - DNA, 18 bp, Artificial Sequence
  SYNTHESIZED
agtggtcctgaaagcatg

SEQ ID NO: 15 - DNA, 20 bp, Artificial Sequence
  SYNTHESIZED
tacaagcaggattacaacac

SEQ ID NO: 16 - protein, 1042 aa, Escherichia coli
ATVNADISYQTFRDFAENKGAFIVGASNINIYDKNGVLVGVLDKAPMPDFSSATMNTGTLPPGDHTLYSPQYVVTAKHVNGSDIMSFGHIQNNYTVVGENNHNSLDIKTRRLNKIVTEVAPAEISSVGAVNGAYQEGGRFKAFYRLGGGLQYIKDKNGNLTPVYTNGGFLTGGTISALSSYNNGQMITAPTGDIFNPANGPLANYLNKGDSGSPLFAYDSLDKKWVLVGVLSSGSEHGNNWVVTTQDFLHQQPKHDFDKTISYDSEKGSLQWRYNKNSGVGTLSQESVVWDMHGKKGGDLNAGKNLQFTGNNGEIILHDSIDQGAGYLQFFDNYTVTSLTDQTWTGGGIITEKGVNVLWQVNGVNDDNLHKVGEGTLTVNGKGVNNGGLKVGDGTVILNQRPDDNGHKQAFSSINISSGRATVILSDANQVNPDKISWGYRGGTLDLNGNNVNFTRLQAADYGAIVSNNNKNKSELTLKLQTLNENDISVDVKTYEVFGGHGSPGDLYYVPASNTYFILKSKAYGPFFSDLDNTNVWQNVGHDRDKAIQIVKQQKIGESSQPYMFHGQLNGYMDVNIHPLSGKDVLTLDGSVNLPEGVITKKSGTLIFQGHPVIHAGMTTSAGQSDWENRQFTMDKLRLDAATFHLSRNAHMQGDISAANGSTVILGSSRVFTDKNDGTGNAVSSVEGSSIATTAGDQSYYSGNVLLENHSSLEVRENFTGGIEAYDSSVSVTSQNAILDHVGSFINSSLLLEKGAKLTAQSGIFTNNTMEIKENASLTLTGIPSVGKPGYYSPVISTTEGIHLGERASLSVKNMGYLSSNIIAEDSAAIINLGDSNATIGKTDSPLFNTLMKGYNAVLQGNIMGPQSSVNMNNALWHSDRNSEIKELKANDSQIELGGRGHFAKLRVKELIASNSVFLVHVNNGQADQLNVTGKLQGSNNTILVNFFNKAANGTNVTLITAPKGSDENTFKAGTQQIGFSNITPEIRTENTDTATKWVLTGYQSVADARASKIATDFMDSGYKSFLTEVNNLNKRMGDLRD

SEQ ID NO: 17 - protein, 1042 aa, Escherichia coli
ATVNADISYQTFRDFAENKGAFIVGASNINIYDKNGVLVGVLDKAPMPDFSSATMNTGTLPPGDHTLYSPQYVVTAKHVNGSDIMSFGHIQNNYTVVGENNHNSLDIKTRRLNKIVTEVAPAEVSSVGAVNGAYQEGGRFTAFYRLGGGLQYIKDKNGNLTPVYTNGGFLTGGTISALNSYNNGQMITAPTGDIFNPANGPLANYLNKGDSGSPLFAYDSLEKKWVLVGVLSSGSEHGNNWVVTTQDFLHQQLKHDFDKTISYDSKKGSLQWRYDKNAGVGTLSQEGVVWDMHGKKGEDLNAGKNLQFTGNNGEVILHDSIDQGAGYLQFFDNYTVTSLTDQTWTGGGIITEKGVNVLWQVNGVNDDNLHKVGEGTLTVNGKGVNNGGLKVGDGTVILNQRPDDNGHKQAFSSINISSGRATVILSDANQVNPDKISWGYRGGTLDLNGNNVTFTRLQAADYGAIVSNNNKNKSELTLKLQTLNENDISVDVKTYEVFGGHGSPGDLYYVSASNTYFILKSKAYGPFFSDLDNTNVWQNVGHDRDKAIQIVKQQKIEESSQPYMFHGQLNGYMDVNIHPLSGKDVLTLDGSVNLPEGVITKKSGTLIFQGHPVIHAGMTTSAGQSDWENRQFTMDKLKLDAATFHLSRNARMQGDISAANGSTVILGSSRVFTDKNDGTGNAVSSVEGSSTATTAADQSYYSGNVLLENHSSLEVRENFTGGIEAYDSSVSVTSQNAILDHVGSFINSSLLLEKGAKLTAQSGIFTNNTMEIKENASLTLTGIPSVGKPGYYSPVISTTEGIHLGERASLSVKNMGYLSSNIIAEDSAAIINLGDSNATIGKTDSPLFNTLMKGYNAVLQGNIMGPQSSVNMNNALWHSDRNSEIKELKANDSQIELGGRGHFAKLRVKELIASNSVFLVHVNNGQADQLNVTGKLQGSNNTILVNFFNKAANGTNVTLITAPKGSDENTFKAGTQQIGFSNITPEIRTENTDTATKWVLTGYQSVADARASKIATDFMDSGYKSFLTEVNNLNKRMGDLRD

SEQ ID NO: 18 - protein, 1042 aa, Escherichia coli
ATVNADISYQTFRDFAENKGAFIVGASNINIYDKNGVLVGVLDKAPMPDFSSATMNTGTLPPGDHTLYSPQYVVTAKHVNGSDIMSFGHIQNNYTVVGENNHNSLDIKTRRLNKIVTEVAPAEVSSVGAVNGAYQEGGRFTAFYRLGGGLQYIKDKNGNLTPVYTNGGFLTGGTISALSSYNNGQMITAPTGDIFNPANGPLANYLNKGDSGSPLFAYDSLEKKWVLIGVLSSGSEYGNNWVVTTQDFLNQQPKHDFDKTISYDSKKGSLQWRYDKDAGVGTLSQEGVVWDMHGKKGEDLNAGKNLQFTGNNGEVILHDSIDQGAGYLQFFNNYTVTSLTDQTWTGGGIITEKGVNVLWQVNGVNNDNLHKVGEGTLTVNGKGVNNGGLKVGDGTVILNQRPDDNGHKQAFSSINISSGRATVILSDANQVNPDKISWGYRGGTLDLNGNNVTFTRLQAADYGAIVSNNNKNKSELTLKLQTLNENDISVDVKTYEFFGGHGSPGDLYYVSASNTYFILKSKAYGPFFSDLNNTNVWQNVGHDRDKAIQIVKQQKIEESSQPYMFHGQLNGYMDVNIHPLSGKDVLTLDGSVNLPEGVITKKSGTLIFQGHPVIHAGMTTSAGQSDWENRQFTMDKLKLDAATFHLSRNARMQGDISAANGSTVILGSSRVFTDKNDGTGNAVSSVEGSSTATTATDQSYYSGNVLLENHSSLEVRENFTGGIEAYDSSVSVTSQNAILDHVGSFINSSLLLEKGAKLTAQSGIFTNNTMEIKENASLTLTGIPSVGKPGYYSPVISTTEGIHLGERASLSVKNMGYLSSNIIAEDSAAIINLGDSNATIGKTDSPLFNTLMRGYNAVLQGNIMGPQSSVNMNNALWHSDRNSEIKELKANDSQIELGGRGHFAKLRVKELIASNSVFLVHVNNGQADQLNVTDKLQGSNNTILVNFFNKAANGTNVTLITAPKGSDENTFKAGTQQIGFSNITPEIRTENTDTATKWVLTGYQSVADARASKIATDFMDSGYKSFLTEVNNLNKRMGDLRD

SEQ ID NO: 19 - protein, 1042 aa, Escherichia coli
ATVNADISYQTFRDFAENKGAFIVGASNINIYDKNGVLVGVLDKAPMPDFSSATMNTGTLPPGDHTLYSPQYVVTAKHVNGSDIMSFGHIQNNYTVVGENNHNSLDIKTRRLNKIVTEVAPAEVSSVGAVNGAYQEGGRFTAFYRLGGGLQYIKDKNGNLTPVYTNGGFLTGGTISALSSYNNGQMITAPTGDIFNPANGPLANYLNKGDSGSPLFAYDSLEKKWVLIGVLSSGSEYGNNWVVTTQDFLNQQPKHDFDKTISYDSKKGSLQWRYDKDAGVGTLSQEGVVWDMHGKKGEDLNAGKNLQFTGNNGEVILHDSIDQGAGYLQFFNNYTVTSLTDQTWTGGGIITEKGVNVLWQVNGVNNDNLHKVGEGTLTVNGKGVNNGGLKVGDGTVILNQRPDDNGHKQAFSSINISSGRATVILSDANQVNPDKISWGYRGGTLDLNGNNVTFTRLQAADYGAIVSNNNKNKSELTLKLQTLNENDISVDVKTYEFFGGHGSPGDLYYVSASNTYFILKSKAYGPFFSDLNNTNVWQNVGHDRDKAIQIVKQQKIEESSQPYMFHGQLNGYMDVNIHPLSGKDVLTLDGSVNLPEGVITKKSGTLIFQGHPVIHAGMTTSAGQSDWENRQFTMDKLKLDAATFHLSRNTRMQGDISATNGSTVILGSSRVFTDKNDGTGNAVSSVEGSSTATTAADQSYYSGNVLLENHSSLEVRENFTGGIEAYDSSVSVTSQNAILDHVGSFINSSLLLEKGAKLTAQSGIFTNNTMEIKENASLTLTGIPSVGKPGYYSPVISTTEGIHLGERASLSVKNMGYLSSNIIAEDSAAIINLGDSNATIGKTDSPLFNTLMRGYNAVLQGNIMGPQSSVNMNNALWHSDRNSEIKELKANDSQIELGGRGHFAKLRVKELIASNSVFLVHVNNGQADQLNVTGKLQGSNNTILVNFFNKAANGTNVTLITAPKGSDENTFKAGTQQIGFSNITPEIRTENTDTATKWVLTGYQSVADARASKIATDFMDSGYKSFLTEVNNLNKRMGDLRD

SEQ ID NO: 20 - protein, 1042 aa, Escherichia coli
ATVNADISYQTFRDFAENKGAFIVGASNINIYDKNGVLVGVLDKAPMPDFSSATMNTGTLPPGDHTLYSPQYVVTAKHVNGSDIMSFGHIQNNYTVVGENNHNSLDIKTRRLNKIVTEVAPAEVSSVGAVNGAYQEGGRFTAFYRLGGGLQYIKDKNGNLTPVYTNGGFLTGGTISALSSYNNGQMITAPTGDIFNPANGPLANYLNKGDSGSPLFAYDSLEKKWVLIGVLSSGSEYGNNWVVTTQDFLNQQQKHDFDKTISYDSKKGSLQWRYDKDAGVGTLSQEGVVWDMHGKKGEDLNAGKNLQFTGNNGEVILHDSIDQGAGYLQFFNNYTVTSLTDQTWTGGGIITEKGVNVLWQVNGVNNDNLHKVGEGTLTVNGKGVNNGGLKVGDGTVILNQRPDDNGHKQAFSSINISSGRATVILSDANQVNPDKISWGYRGGTLDLNGNNVTFTRLQAADYGAIVSNNNKNKSELTLKLQTLNENDISVDVKTYEVFGGRGSPGDLYYVSASNTYFILKSKAYGPFFSDLDNTNVWQNVGHDRDKAIQIVKQQKIEESSQPYMFHGQLNGYMDVNIHPLSGKDVLTLDGSVNLPEGVITKKSGTLIFQGHPVIHAGMTTSAGQSDWENRQFTMDKLKLDAATFHLSRNTRMQGDISAANGSTVILGSSRVFTDKNDGTGNAVSSVEGSSTATTAADQSYYSGNVLLENHSSLEVRENFTGGIEAYDSSVSVTSQNAILDHVGSFINSSLLLEKGAKLTAQSGIFTNNTMEIKENASLTLTGIPSVGKPGYYSPVISTTEGIHLGERASLSVKNMGYLSSNIIAEDSAAIINLGDSNATIGKTDSPLFNTLMRGYNAVLQGNIMGPQSSVNMNNALWHSDRNSEIKELKANDSQIELGGRGHFAKLRVKELIASNSVFLVHVNNGQADQLNVTGKLQGSNNTILVNFFNKAANGTNVTLITAPKGSDENTFKAGTQQIGFSNITPEIRTENTDTATKWVLTGYQSVADARASKIATDFMDSGYKSFLTEVNNLNKRMGDLRD

SEQ ID NO: 21 - protein, 1042 aa, Escherichia coli
ATVNADISYQTFRDFAENKGAFIVGASNINIYDKNGVLVGVLDKAPMPDFSSATMNTGTLPPGDHTLYSPQYVVTAKHVNGSDIMSFGHIQNNYTVVGENNHNSLDIKTRRLNKIVTEVAPAEVSSVGAVNGAYQEGGRFTAFYRLGGGLQYIKDKNGNLTPVYTNGGFLTGGTISALNSYNNGQMITAPTGDIFNPANGPLANYLNKGDSGSPLFAYDSLEKKWVLVGVLSSGSEHGNNWVVTTQDFLHQQLKHDFDKTISYDSKKGSLQWRYDKNAGVGTLSQEGVVWDMHGKKGEDLNAGKNLQFTGNNGEVILHDSIDQGAGYLQFFDNYTVTSLTDQTWTGGGIITEKGVNVLWQVNGVNDDNLHKVGEGTLTVNGKGVNNGGLKVGDGTVILNQRPDDNGHKQAFSSINISSGRATVILSDANQVNPDKISWGYRGGTLDLNGNNVTFTRLQAADYGAIVSNNNKNKSELTLKLQTLNENDISVDVKTYEVFGGHGSPGDLYYVPASNTYFILKSKAYGPFFSDLDNTNVWQNVGHDRDKAIQIVKQQKIEESSQPYMFHGQLNGYMDVNIHPLSGKDVLTLDGSVNLPEGVITKKSGTLIFQGHPVIHAGMTTSAGQSDWENRQFTMDKLKLDAATFHLSRNARMQGDISAANGSTVILGSSRVFTDKNDGTGNAVSSVEGSSTATTAADQSYYSGNVLLENHSSLEVRENFTGGIEAYDSSVSVTSQNAILDHVGSFINSSLLLEKGAKLTAQSGIFTNNTMEIKENASLTLTGIPSVGKPGYYSPVISTTEGIHLGERASLSVKNMGYLSSNIIAEDSAAIINLGDSNATIGKTDSPLFNTLMKGYNAVLQGNIMGPQSSVNMNNALWHSDRNSEIKELKANDSQIELGGRGHFAKLRVKELIASNSVFLVHVNNGQADQLNVTDKLQGSNNTILVNFFNKAANGTNVTLITAPKGSDENTFKAGTQQIGFSNITPEIRTENTDTATKWVLTGYQSVADARASKIATDFMDSGYKSFLTEVNNLNKRMGDLRD

SEQ ID NO: 22 - protein, 1042 aa, Escherichia coli
ATVNADISYQTFRDFAENKGAFIVGASNINIYDKNGVLVGVLDKAPMPDFSSATMNTGTLPPGDHTLYSPQYVVTAKHVNGSDIMSFGHIQNNYTVVGENNHNSLDIKTRRLNKIVTEVAPAEVSSVGAVNGAYQEGGRFKAFYRLGGGLQYIKDKNGNLTPVYTNGGFLTGGTISALSSYNNGQMITAPTGDIFNPANGPLANYLNKGDSGSPLFAYDSLDKKWVLVGVLSSGSEHGNNWVVTTQDFLHQQPKHDFDKTISYDSEKGSLQWRYNKNSGVGTLSQESVVWDMHGKKGGDLNAGKNLQFTGNNGEIILHDSIDQGAGYLQFFDNYTVTSLTDQTWTGGGIITEKGVNVLWQVNGVNDDNLHKVGEGTLTVNGKGVNNGGLKVGDGTVILNQRPDDNGHKQAFSSINISSGRATVILSDANQVNPDKISWGYRGGTLDLNGNNVNFTRLQAADYGAIVSNNNKNKSELTLKLQTLNENDISVDVKTYEVFGGHGSPGDLYYVPASNTYFILKSKAYGPFFSDLDNTNVWQNVGHDRDKAIQIVKQQKIGESSQPYMFHGQLNGYMDVNIHPLSGKDVLTLDGSVNLPEGVITKKSGTLIFQGHPVIHAGMTTSAGQSDWENRQFTMDKLRLDAATFHLSRNAHMQGDISAANGSTVILGSSRVFTDKNDGTGNAVSSVEGSSIATTAGDQSYYSGNVLLENHSSLEVRENFTGGIEAYDSSVSVTSQNAIFDHVGSFVNSSLLLEKGAKLTAQSGIFTNNTMKIKENASLTLTGIPSVGKPGYYSPVTSTTEGIHLGERASLSVKNMGYLSSNITAENSAAIINLGDSNATIGKTDSPLFSTLMRGYNAVLQGNIMGPQSSVNMNNALWHSDRNSELKELKANDSQIELGVRGHFAKLRVKELIASNSVFLVHANNSQADQLNVTDKLQGSNNTILVDFFNKAANGTNVTLITAPKGSDENTFKAGTQQIGFSNITPEIRTENTDTATQWVLTGYQSVADARASKIATDFMDSGYKSFLTEVNNLNKRMGDLRD

SEQ ID NO: 23 - protein, 1042 aa, Escherichia coli
ATVNADISYQTFRDFAENKGAFIVGASNINIYDKNGVLVGVLDKAPMPDFSSATMNTGTLPPGDHTLYSPQYVVTAKHVNGSDIMSFGHIQNNYTVVGENNHNSLDIKIRRLNKIVTEVAPAEISSVGAVNGAYQEGGRFKAFYRLGGGLQYIKDKNGNLTPVYTNGGFLTGGTISALSSYNNGQMITAPTGDIFNPANGPLANYLNKGDSGSPLFAYDSLDKKWVLVGVLSSGSEHGNNWVVTTQDFLHQQPKHDFDKTISYDSEKGSLQWRYNKNSGVGTLSQESVVWDMHGKKGGDLNAGKNLQFTGNNGEIILHDSIDQGAGYLQFFDNYTVTSLTDQTWTGGGIITEKGVNVLWQVNGVNDDNLHKVGEGTLTVNGKGVNNGGLKVGDGTVILNQRPDDNGHKQAFSSINISSGRATVILSDANQVNPDKISWGYRGGTLDLNGNNVNFTRLQAADYGAIVSNNNKNKSELTLKLQTLNENDISVDVKTYEVFGGHGSPGDLYYVPASNTYFILKSKAYGPFFSDLDNTNVWQNVGHDRDKAIQIVKQQKIGESSQPYMFHGQLNGYMDVNIHPLSGKDVLTLDGSVNLPEGVITKKSGTLIFQGHPVIHAGMTTSAGQSDWENRQFTMDKLRLDAATFHLSRNAHMQGDISAANGSTVILGSSRVFTDKNDGTGNAVSSVEGSSIATTAGDQSYYSGNVLLENHSSLEVRENFTGGIEAYDSSVSVTSQNAILDHVGSFINSSLLLEKGAKLTAQSGIFTNNTMEIKENASLTLTGIPSVGKPGYYSPVISTTEGIHLGERASLSVKNMGYLSSNIIAEDSAAIINLGDSNATIGKTDSPLFNTLMKGYNAVLQGNIMGPQSSVNMNNALWHSDRNSEIKELKANDSQIELGGRGHFAKLRVKELIASNSVFLVHVNNGQADQLNVTGKLQGSNNTILVNFFNKAANGTNVTLITAPKGSDENTFKAGTQQIGFSNITPEIRTENTDTATKWVLTGYQSVADARASKIATDFMDSGYKSFLTEVNNLNKRMGDLRD

SEQ ID NO: 24 - protein, 1044 aa, Escherichia coli
ATVNADISYQTFRDFAENKGAFIVGASNINIYDKNGVLVGVLDKAPMPDFSSATMNTGTLPPGDHTLYSPQYVVTAKHVNGSDIMSFGHIQNNYTVVGENNHNSLDIKTRRLNKIVTEVAPAEVSSVGAVNGAYQEGGRFTAFYRLGGGLQYIKDKNGNLTPVYTNGGFLTGGTISALSSYNNGQMITAPTGDIFNPANGPLANYLNKGDSGSPLFAYDSLEKKWVLIGVLSSGSEYGNNWVVTTQDFLNQQQKHDFDKTISYDSKKGSLQWRYDKDAGVGTLSQEGVVWDMHGKKGEDLNAGKNLQFTGNNGEVILHDSIDQGAGYLQFFNNYTVTSLTDQTWTGGGIITEKGVNVLWQVNGVNNDNLHKVGEGTLTVNGKGVNNGGLKVGDGTVILNQRPDDNGHKQAFSSINISSGRATVILSDANQVNPDKISWGYRGGTLDLNGNNVTFTRLQAADYGAIVSNNNNNKNKSELTLKLQTLNENDISVDVKTYEVFGGRGSPGDLYYVSASNTYFILKSKAYGPFFSDLDNTNVWQNVGHDRDKAIQIVKQQKIEESSQPYMFHGQLNGYMDVNIHPLSGKDVLTLDGSVNLPEGVITKKSGTLIFQGHPVIHAGMTTSAGQSDWENRQFTMDKLKLDAATFHLSRNTRMQGDISAANGSTVILGSSRVFTDKNDGTGNAVSSVEGSSTATTAADQSYYSGNVLLENHSSLEVRENFTGGIEAYDSSVSVTSQNAILDHVGSFINSSLLLEKGAKLTAQSGIFTNNTMEIKENASLTLTGIPSVGKPGYYSPVISTTEGIHLGERASLSVKNMGYLSSNIIAEDSAAIINLGDSNATIGKTDSPLFNTLMRGYNAVLQGNIMGPQSSVNMNNALWHSDRNSEIKELKANDSQIELGGRGHFAKLRVKELIASNSVFLVHVNNGQADQLNVTGKLQGSNNTILVNFFNKAANGTNVTLITAPKGSDENTFKAGTQQIGFSNITPEIRTENTDTATKWVLTGYQSVADARASKIATDFMDSGYKSFLTEVNNLNKRMGDLRD

SEQ ID NO: 25 - protein, 1042 aa, Artificial Sequence
  SYNTHESIZED
ATVNADISYQTFRDFAENKGAFIVGASNINIYDKNGVLVGVLDKAPMPDFSSATMNTGTLPPGDHTLYSPQYVVTAKHVNGSDIMSFGHIQNNYTVVGENNHNSLDIKTRRLNKIVTEVAPAEVSSVGAVNGAYQEGGRFTAFYRLGGGLQYIKDKNGNLTPVYTNGGFLTGGTISALSSYNNGQMITAPTGDIFNPANGPLANYLNKGDSGSPLFAYDSLEKKWVLVGVLSSGSEHGNNWVVTTQDFLHQQPKHDFDKTISYDSKKGSLQWRYDKNAGVGTLSQEGVVWDMHGKKGEDLNAGKNLQFTGNNGEVILHDSIDQGAGYLQFFDNYTVTSLTDQTWTGGGIITEKGVNVLWQVNGVNDDNLHKVGEGTLTVNGKGVNNGGLKVGDGTVILNQRPDDNGHKQAFSSINISSGRATVILSDANQVNPDKISWGYRGGTLDLNGNNVTFTRLQAADYGAIVSNNNKNKSELTLKLQTLNENDISVDVKTYEVFGGHGSPGDLYYVSASNTYFILKSKAYGPFFSDLDNTNVWQNVGHDRDKAIQIVKQQKIEESSQPYMFHGQLNGYMDVNIHPLSGKDVLTLDGSVNLPEGVITKKSGTLIFQGHPVIHAGMTTSAGQSDWENRQFTMDKLKLDAATFHLSRNARMQGDISAANGSTVILGSSRVFTDKNDGTGNAVSSVEGSSTATTAADQSYYSGNVLLENHSSLEVRENFTGGIEAYDSSVSVTSQNAILDHVGSFINSSLLLEKGAKLTAQSGIFTNNTMEIKENASLTLTGIPSVGKPGYYSPVISTTEGIHLGERASLSVKNMGYLSSNIIAEDSAAIINLGDSNATIGKTDSPLFNTLMRGYNAVLQGNIMGPQSSVNMNNALWHSDRNSEIKELKANDSQIELGGRGHFAKLRVKELIASNSVFLVHVNNGQADQLNVTGKLQGSNNTILVNFFNKAANGTNVTLITAPKGSDENTFKAGTQQIGFSNITPEIRTENTDTATKWVLTGYQSVADARASKIATDFMDSGYKSFLTEVNNLNKRMGDLRD

SEQ ID NO: 26 - protein, 600 aa, Escherichia coli
MNRIYKLKFDKRRNELVVVSEITTGVGNAKATGSVEGEKSPRRGVRAMALSLLSGMMIMAHPAMSANLPTGGQIVAGSGSIQTPSGNQMNIHQNSQNMVANWNSFDIGKGNTVQFYQPSSSAVALNRVVGGGESQIMGNLKANGQVFLVNPNGVLFGKGASVSTSGFVASTRDIKNDDFMNRRYTFSGGQKAGAAIVNQGALTTNAGGYIVLAADRVSNSGTIRTPGGKTVLAASERVTLQLDNGGLMSAQVTGDVVNALVENRGLVSARDGQVYLTALGRDMLMNTVLNVSGVVEASGMHRQDGNIVLDGGDSGVVHLSSTLQADNASGQGGKVVVQGKNILLDKGSSITATGGQGGGEVYVGGGWQGKDSNIRNADKVVMQDGARIDVSATQQGNGGTAVLWSDSYTNFHGQISAKGGETGGNGGQVETSSHGNLQAFGTVSASAKKGKAGNWLLDSADITIVNGSNDSNVSKTETTQSPPHTQFAPTAAGSAVSNTSINNRLNNGTSVTILASHTAKDGAQKGNITVNAAINKSNGSDANLTLQAGGNITVNNSITSTEGKLNVNLSGARTSNGSITISNNANITTNGGDITVGTTN

SEQ ID NO: 27 - protein, 600 aa, Escherichia coli
MNRIYKLKFDKRRNELVVVSEITTGVGNAKATGSVEGEKSPRRGVRAMALSLLSGMMIMAHPAMSANLPTGGQIVAGSGSIQTPSGNQMNIHQSSQNMVANWNSFDIGKGNTVQFYQPNSSAVALNRVVGGGESQIMGNLKANGQVFLVNPSGVLFGKGASVSTSGFVASTRDIKNDDFMNRRYTFSGGQKAGAAIVNQGALTTNAGGYIVLAADRVSNSGTIRTPGGKTVLAASERVTLQLDNGGLMSAQVTGDVVNALVENRGLVSARDGQVYLTALGRDMLMNTVLNVSGVVEAGGMHRQDGNIVLDGGDSGVVHLSGTLQADNASGQGGKVVVQGKNILLDKGSSITATGGQGGGEVYVGGGWQGKDSNIRNADKVVMQDGARIDVSATQQGNGGTAVLWSDSYTNFHGQISAKGGETGGNGGQVETSSHGNLQAFGTVSASAKKGKAGNWLLDSADITIVNGSNDSNVSKTETTQSPPHTQFAPTAAGSAVSNTSINNRLNNGTSVTIRTGHTTKDGAQKGNITVNAAINKSNGSDANLTLQAGGNITVNNSITSTSGKLNVNLSGANTNNGSITISNNVNITTSGGSITIGTAN

SEQ ID NO: 28 - protein, 600 aa, Escherichia coli
MNRIYKLKFDKRRNELVVVSEITTGVGNAKATGSVEGEKSPRRGVRAMALSLLSGMMIMAHPAMSANLPTGGQIVAGSGSIQTPSGNQMNIHQSSQNMVANWNSFDIGKGNTVQFYQPDSSAVALNRVVGGGESQIMGNLKANGQVFLVNPSGVLFGKGASVSTSGFVASTRDIKNDDFMNRRYTFSGGQKAGAAIVNQGALTTNAGGYIVLAADRVSNSGTIRTPGGKTVLAASERVTLQLDNGGLMSAQVTGDVVNALVENRGLVSARDGQVYLTALGRDMLMNTVLNVSGVVEAGGMHRQDGNIVLDGGDSGVVHLSGTLQADNASGQGGKVVVQGKNILLDKGSSITATGGQGGGEVYVGGGWQGKDSNIRNADKVVMQDGARIDVSATQQGNGGTAVLWSDSYTNFHGQISAKSGETGGNGGQVETSSHGNLQAFGTVSASAKKGKAGNWLLDSADITIVNGSNDSNVSKTETTQSPPHTQFAPTAAGSAVSNTSINNRLNNGTSVTILTSHTTKDGAQKGNITVNAAINKSNGSDANLTLQAGGNITVNNSITSTSGKLNVNLSGANTNNGSITISNNVNITTSGGSITIGTAN

SEQ ID NO: 29 - protein, 600 aa, Escherichia coli
MNRIYKLKFDKRRNELVVVSEITTGVGNAKATGSVEGEKSPRRGVRAMALSLLSGMMIMAHPAMSANLPTGGQIVAGSGSIQTPSGNQMNIHQSSQNMVANWNSFDIGKGNTVQFYQPDSSAVALNRVVGGGESQIMGNLKANGQVFLVNPNGVLFGKEASVSTSGFVASTRDIKNDDFMNRRYTFSGGQKAGAEIVNQGALTTNAGGYIVLTADRVSNSGTIRTPGGKTVLAASERVTLQLDNGGLMSVQVTGDVVNALVENRGLVSARDGQVYLTALGRDMLMNTVLNVSGVVEAGGMRRQDGNIVLDGGDSGVVHLSGTLQADNASGQGGKVVVQGKNILLDKGSSITATGGQGGGEVYVGGGWQGKDSNIRNADKVVMQDGARIDVSATQQGNGGTAVLWSDSYTNFHGQISAKGGETGGNGGQVETSSQGNLQAFGTVSASAKKGKAGNWLLDSADITIVNGSNDSNDSKTETTQSPPHTQFAPTAAGSAVSNTSINNRLNNGTSVTILTSHTAKDGAQKGNITVNAAINKSNGSDASLTLQAGGNITVNNSITSTEGKLNVNLSGARTSNGSITISNNANITTNGGDITVGTTN

SEQ ID NO: 30 - protein, 600 aa, Escherichia coli
MNRIYKLKFDKRRNELVVVSEITTGVSNAKATGSVEGEKSPRRGVRAMALSLLSGMMIMAHPAMSANLPTGGQIVAGSGSIQTPSGNQMNIHQNSQNMVANWNSFDIGKGNTVQFDQPSSSAVALNRVVGGGESQIMGNLKANGQVFLVNPNGVLFGEGASVSTSGFVASTRDIKNDDFMNRRYTFSGGQKAGAAIVNQGELTTNAGGYIVLAADRVSNSGTIRTPGGKTVLAASERITLQLDNGGLMSVQVTGDVVNALVENRGLVSARDGQVYLTALGRGMLMNTVLNVSGVVEASGMHRQDGNIVLDGGDSGVVHLSGTLQADNASGQGGKVVVQGKNILLDKGSNITATGGQGGGEVYVGGGWQGKDSNIRNADKVVMQGGARIDVSATQQGNGGTAVLWSDSYTNFHGQISAKGGETGGNGGRVETSSHGNLQAFGTVSASAKKGKAGNWLLDSADITIVNGSNVSKTETTQSPPHTQFAPTAAGSAVSNTSINNRLNNGTSVTILTHRTRTGTAQGGNITVNAAINKSNGSDVNLTLQAGGNITVNSSITSTNGKLNVNLSGARTSNGSITISNNANITTNGGDITVGTTNTSN

SEQ ID NO: 31 - protein, 600 aa, Escherichia coli
MNRIYKLKFDKRRNELVVVSEITTGVGNAKATGSVEGEKSPRRGVRAMALSLLSGMMIMAHPAMSANLPTGGQIVAGSGSIQTPSGNQMNIHQNSQNMVANWNSFDIGKGNTVQFDQPSSSAVALNRVVGGGESQIMGNLKANGQVFLVNPNGVLFGEGASVSTSGFVASTRDIKNDDFMNRRYTFSGGQKAGAAIVNQGELTTNAGGYIVLAADRVSNSGTIRTPGGKTVLAASERITLQLDNGGLMSVQVTGDVVNALVENRGLVSARDGQVYLTALGRGMLMNTVLNVSGVVEASGMHRQDGNIVLDGGDSGVVHLSGTLQADNASGQGGKVVVQGKNILLDKGSNITATGGQGGGEVYVGGGWQGKDSNIRNADKVVMQGGARIDVSATQQGNGGTAVLWSDSYTNFHGQISAKGGETGGNGGRVETSSHGNLQAFGTVSASAKKGKAGNWLLDSADITIVNGSNVSKTETTQSPPHTQFAPTAAGSAVSNTSINNRLNNGTSVTILTHRTRTGTAQGGNITVNAAINKSNGSDVNLTLQAGGNITVNSSITSTNGKLNVNLSGARTSNGSITISNNANITTNGGDITVGTTNTSN

SEQ ID NO: 32 - protein, 413 aa, Escherichia coli
MNRIYKLKFDKRRNELVVVSEITTGVSNAKATGSVEGEKSPRRGVRAMALSLLSGMMIMAHPAMSANLPTGGQIVAGSGSIQTPSGNQMNIHQNSQNMVANWNSFDIGKGNTVQFDQPSSSAVALNRVVGGGESQIMGNLKANGQVFLVNPNGVLFGEGASVSTSGFVASTRDIKNDDFMNRRYTFSGGQKAGAAIVNQGELTTNAGGYIVLAADRVSNSGTIRTPGGKTVLAASERITLQLDNGGLMSVQVTGDVVNALVENRGLVSARDGQVYLTALGRGMLMNTVLNVSGVVEASGMHRQDGNIVLDGGDSGVVHLSGTLQADNASGQGGKVVVQGKNILLDKGSNITATGGQGGGEVYVGGGWQGKDSNIRNADKVVMQGGARIDVSATQQGNGGTAVLWSDSYTNFHG